(19) United States
(12) Patent Application Publication
(10) Pub. No.: US 2025/0051416 A1
(43) Pub. Date: Feb. 13, 2025

(54) INSULIN RECEPTOR PARTIAL AGONISTS

(71) Applicant: Merck Sharp & Dohme LLC, Rahway, NJ (US)

(72) Inventors: Danqing Feng, Green Brook, NJ (US); Pei Huo, Millburn, NJ (US); Ahmet Kekec, Hoboken, NJ (US); Songnian Lin, Holmdel, NJ (US); Dmitri A. Pissarnitski, Scotch Plains, NJ (US); Lin Yan, East Brunswick, NJ (US)

(73) Assignee: Merck Sharp & Dohme LLC, Rahway, NJ (US)

(21) Appl. No.: 18/041,821

(22) PCT Filed: Aug. 24, 2021
(86) PCT No.: PCT/US2021/047313

§ 371 Date: Feb. 16, 2023

Related U.S. Application Data
(60) Provisional application No. 63/070,377, filed on Aug. 26, 2020.

(51) Int. Cl. C07K 14/62; A61K 45/06; A61K 47/64; A61P 3/10

(52) U.S. Cl. CPC C07K 14/62; A61K 45/06; A61K 47/64; A61P 3/10

(57) ABSTRACT

Insulin dimers and insulin analog dimers that act as partial agonists at the insulin receptor are disclosed.

### BACKGROUND OF THE INVENTION

### Field of the Invention

The present invention relates to insulin dimers and insulin analog dimers that act as partial agonists at the insulin receptor.

### Description of Related Art

Insulin is the most effective anti-diabetic therapy for glycemic control in diabetic patients, but high hypoglycemia risk limits its treatment efficacy. The key reason that diabetic patients on insulin are not attaining their HbA1C goal is because administration doses are often intentionally lowered to avoid potentially life-threatening hypoglycemia. To improve the narrow therapeutic index (TI) of insulin may allow for further lowering glucose level to attain glycemic control with lower hypoglycemic risk, reducing health care cost associated with hypoglycemia treatment. Among various active research areas of developing insulins with improved TI, covalently linked insulin dimers have been reported in the literature to function as partial agonists of insulin receptor. It is believed that partial agonism of the insulin receptor by these covalent dimers may elicit a desired submaximal activation of the insulin receptor while it may also reduce overactivation of the insulin receptor by excess amount of the endogeneous insulin, leading to an increased therapeutic index in vivo.

Insulin is an essential therapy for type 1 diabetes mellitus (T1DM) patients and many type 2 mellitus diabetics (T2DMs), prescribed to close to one third of U.S. patients among all anti-diabetic drug users in the past decade. The worldwide market for insulins was US$20.4 billion in 2013 and is growing at a faster rate than all other anti-diabetic agents combined. However, challenges of current insulin therapies, including narrow TI to hypoglycemia and body weight gain, limit their wider adoption and potential for patients to achieve ideal glycemic control.

In addition to prandial insulin secretion in response to meals, the pancreas releases insulin at a “basal” rate, governed largely by plasma glucose levels to maintain appropriate fasting glucose regulation. This is achieved mainly by controlling hepatic glucose release, through endogenous insulin's hepato-preferring action. Modern insulin analogs include rapid acting and basal insulins, as well as mixtures of these two. Rapid-acting insulin analogs (RAA) are developed to control post-prandial hyperglycemia while insulins with extended duration of action regulate basal glucose levels. Long-acting insulins are used by all T1DM (in combination with prandial injections) and the majority of T2DM patients start their insulin therapy from a basal product. Basal insulin consumption is growing rapidly as the worldwide diabetes population (particularly T2DM) soars.

Insulin dimers have been disclosed in Brandenburg et al. in U.S. Pat. No. 3,907,763 (1973); Tatnell et al., Biochem J. 216: 687-694 (1983); Shüttler and Brandenburg, Hoppe-Seyler's Z. Physiol. Chem, 363, 317-330, 1982; Weiland et al., Proc Natl. Acad. Sci. (USA) 87: 1154-1158 (1990); Deppe et al., Naunyn-Schmiedeberg's Arch Pharmacol (1994) 350:213-217; Brandenburg and Havenith in U.S. Pat. No. 6,908,897(B2) (2005); Knudsen et al., PLOS ONE 7: e51972 (2012); DiMarchi et al in WO2011/159895; DiMarchi et al. in WO 2014/052451; and Herrera et al., WO2014141165. Insulin dimers have also been disclosed in wO2016081670 and WO2017205309 More recently, insulin dimers have been described in Brant—Synthesis and Characterization of Insulin Receptor Partial Agonists as a Route to Improved Diabetes Therapy, Ph.D. Dissertation, Indiana University (April 2015) and Zaykov and DiMarchi, Poster P212—Exploration of the structural and mechanistic basis for partial agonism of insulin dimers, American Peptide Symposium, Orlando FL (June 20-25 (2015).

Despite continuous development efforts over the past several decades, available long-acting insulins are still not optimized compared to physiological basal insulin. This is partially because major focus was on improving PK flatness of these analogs but not fixing the relative over-insulinization of peripheral tissues, which contributes to increased hypoglycemia risk. As a result, hypoglycemia remains a key medical risk with huge burden on patients and causes significant morbidity and mortality.

### REFERENCE TO SEQUENCE LISTING SUBMITTED ELECTRONICALLY

The sequence listing of the present application is submitted electronically via EFS-Web as an ASCII formatted sequence listing with a file name 25087USPCT-SEQLIST-08JAN2024.txt, creation date of Jan. 8, 2024, and a size of 6,406 bytes. This sequence listing submitted via EFS-Web is part of the specification and is herein incorporated by reference in its entirety.

### BRIEF SUMMARY OF THE INVENTION

The present invention provides compounds comprising two insulin molecules covalently linked to form an insulin molecule dimer that may activate the insulin receptor with regular insulin-like potency but with reduced maximum activity. These compounds are insulin receptor partial agonists (IPRAs): they behave like other insulin analogs to lower glucose effectively but with lower risk of hypoglycemia.

Provided are insulin receptor partial agonist covalent insulin dimers formulated as novel and transformative basal insulins (once daily administration) that manifest an improved therapeutic index (TI) over current standard of care (SOC) basal insulins. In one embodiment, the IPRAs of the present invention may lower glucose effectively with reduced risk of hypoglycemia in diabetic minipig and has the property of a once daily (QD) basal insulin. The improved TI may empower practitioners to more aggressively dose IRPAs of the present invention to achieve target goals for control of fasting glucose. Tight control of fasting glucose and HbA1c by an IRPA may allow it to serve as 1) a stand-alone long-acting insulin with an enhanced efficacy and safety profile in T2DM and 2) an improved foundational basal insulin in T1DM (and some T2DM) for use with additional prandial rapid-acting insulin analogs (RAA) doses. Thus, the present invention provides the following embodiments.

The present invention provides an insulin dimer comprising a first B29 Lys of a first insulin heterodimer molecule having a first A-chain polypeptide and first B-chain polypeptide and a second B29 Lys of a second insulin heterodimer having a second A-chain polypeptide and second B-chain polypeptide conjugated together by a bifunctional linker moiety selected from the group consisting Linker 1, Linker 2, Linker 3, Linker 4, Linker 5, Linker 6, Linker 7, Linker 8, Linker 9, Linker 10, Linker 11, Linker 12, Linker 13, Linker 14, Linker 15, and Linker 16.

In a further aspect of the insulin dimer, the first and second insulin or insulin analog heterodimers are the same or wherein the first and second insulin or insulin analog heterodimers are different.

In particular aspects of the insulin dimer, the linker moiety covalently links the first insulin or insulin analog heterodimer and the second insulin or insulin analog heterodimer via the epsilon amino group of a lysine residue at or near the carboxy terminus of their respective B-chain polypeptides.

In particular aspects of the insulin dimer, the first or second B-chain polypeptides are conjugated at its N-terminal amino acid with a capping group or the N-terminal amino acids of both B-chain polypeptides of the first insulin heterodimer and second insulin heterodimer are conjugated with a capping group. A subembodiment of this aspect of the invention is realized when the first insulin heterodimer and second insulin heterodimer are conjugated at B29 and B29′ of the insulin dimer by bifunctional linker moiety selected from the group consisting Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, and Linker 16.

In particular aspects of the insulin dimer, the B-chain polypeptide is conjugated at its N-terminal amino acid to a capping group, or at least the N-terminal amino acid of the first insulin heterodimer molecule is conjugated to a capping group or the N-terminal amino acids of both the first insulin heterodimer and second insulin heterodimer are conjugated to a capping group.

In particular aspects of the insulin dimer, the capping group comprises capping group is a linear or branch C₁₋₆ alkyl, or N-hydroxysuccinimide ester linked to a group having the general formula RC(O)—, where R is:
a) a peptide,
b) PEG,
c) linear or branched C₁₋₆ alkyl chain,
d) R′NH—, or
e) R′O—,
wherein R′ is H (when R is R′NH—), peptide, PEG, or linear or branched alkyl chain, and wherein each said peptide, PEG and linear or branched alkyl may be unsubstituted or substituted with 1 to 3 groups selected from amino-, phosphono-, hydroxy-, carboxylic acid, amino acid, PEG, and saccharides.

In aspects of this invention the capping group is, for example dimethyl or isobutyl, or is a group RC(O) that may be exemplified as acetyl, phenylacetyl, methoxy acetyl, 2-(carboxymethoxy)acetyl, 2-[bis(carboxymethylamino)]acetyl, carbamoyl, N-alkyl carbamoyl, glutaryl, trifluoroacetyl, glycyl, aminoethylglucose (AEG), AEG-C6, PEG (e.g., PEG1, PEG2, PEG3, PEG4, PEG5, PEG8, PEG24,), and alkoxy carbonyl.

In particular aspects of the insulin dimer, the capping group is is a linear or branch C₁₋₆ alkyl. As aspect of this invention is realized when the alkyl is dimethyl (Me2) or isobutyl.

In particular aspects of the insulin dimer, the capping group is glutaryl, Me2, carbamoyl, or 2,5,8,11,14,17,20,23-octaoxahexacosan-26-yl. In another subembodiment of this aspect of the invention, the capping group is carbamoyl.

In particular aspects of the insulin dimer, the capping group is acetyl or 2-(carboxymethoxy) acetyl. A subembodiment of this aspect of the invention is realized when the capping group is acetyl. Another subembodiment of this aspect of the invention is realized when the capping group is 2-(carboxymethoxy)acetyl.

In particular aspects of the insulin dimer, the capping group is selected from capping groups Capping Group No. 1, 2, 3, 4, 5, 6, 7, and 8 in Table II.

In particular aspects of the insulin dimer, the first insulin and the second insulin heterodimers are independently native human insulin, insulin lispro, insulin aspart, desB30 insulin, or insulin glargine.

The present invention further provides a composition comprising a first insulin or insulin analog heterodimer and a second insulin or insulin analog heterodimer each heterodimer including an A-chain polypeptide and a B-chain polypeptide, wherein the A-chain polypeptide and the B-chain polypeptide are linked together through interchain disulfide bonds; wherein the first and second insulin or insulin analog heterodimers are covalently linked together through a linking moiety joining the side chain of an amino acid at or near the carboxy terminus of the two respective B-chain polypeptides; wherein the linking moiety is selected from the group consisting of Linking moiety 1, 2, 3, 4, 5, 6, 7, 8, 9, 10, 11, 12, 13,14, 15, and 16; wherein the amino terminus of the B-chain polypeptides of the first insulin polypeptide and second insulin polypeptide is covalently linked to a capping group. An embodiment of this aspect of the invention is realized when the capping group is RC(O)—, where R can be peptide, PEG, linear or branched alkyl chain, said peptide, PEG and alkyl optionally substituted with amino-, phosphono-, hydroxy-, carboxylic acid, amino acid, PEG, and saccharides, or R can be R′NH, or R′O, wherein R′ can be H (when R is R′NH), peptide, PEG, linear or branched alkyl chain, said peptide, PEG and alkyl optionally substituted with amino-, phosphono-, hydroxy-, or, carboxylic acid, amino acid, PEG, and saccharides. Another embodiment of this aspect of the invention is realized when RC(O) is a capping group that may be exemplified as acetyl, phenylacetyl, isobutyl, methoxy acetyl, 2-(carboxy methoxy) acetyl, 2-[bis(carboxymethylamino)]acetyl, carbamoyl, N-alkyl carbamoyl, glutaryl, trifluoroacetyl, glycyl, aminoethylglucose (AEG), AEG-C6, PEG (e.g., PEG1, PEG2, PEG3, PEG4, PEG5, PEG8, PEG24,), N-dimethyl, and alkoxycarbonyl. Yet in another embodiment, the capping group of the insulin dimer is Capping Group No. 1, 2, 3, 4, 5, 6, 7, and 8 in Table II. Still in another embodiment the capping group of the insulin dimer is selected from glutaryl, Me2, carbamoyl, or 2,5,8,11,14,17,20,23-octaoxahexacosan-26-yl. Still in another embodiment the capping group of the insulin dimer is carbamoyl. In another embodiment the capping group of the insulin dimer is acetyl or 2-(carboxymethoxy)acetyl. In another embodiment the capping group of the insulin dimer is acetyl. In another embodiment the capping group of the insulin dimer 2-(carboxymethoxy)acetyl. Still another embodiment of this aspect of the invention is realized when the insulin is recombinant human insulin and the insulin analog is selected from the group consisting of insulin lispro, insulin aspart, and insulin glargine.

In particular aspects of the insulin dimer, the first and second insulin or insulin analog heterodimers are the same or wherein the first and second insulin or insulin analog heterodimers are different.

Exemplary insulin dimers of the present invention are represented by Formula I:

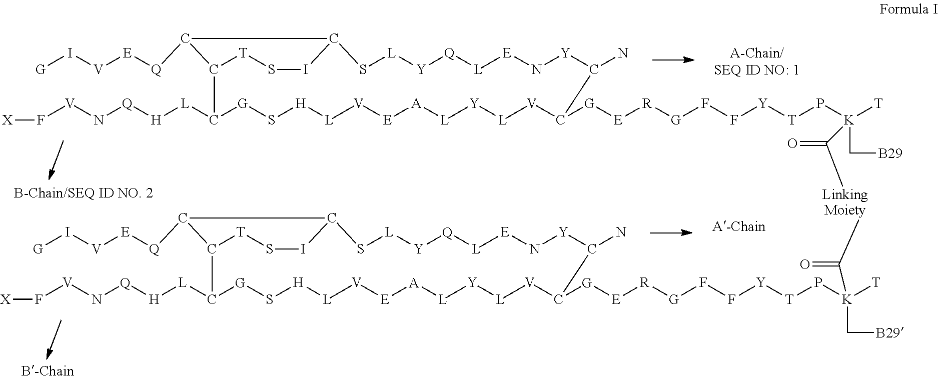

wherein at least one of B and B′ sites are blocked by small capping groups represented by X, a first insulin heterodimer molecule having a first A-chain polypeptide and first B-chain polypeptide and a second insulin heterodimer having a second A′-chain polypeptide and second B′-chain polypeptide that is conjugated together at the B29 and B29′of the first and second heterodimer, respectively, by a bifunctional linking group represented by Linker, the A-chain and A′-chain peptides have the amino acid sequence shown in SEQ ID NO: 1 and the B-chain and B′-chain peptides have the amino acid sequence shown in SEQ ID NO: 2, and wherein the cysteine residues at positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond.

The present invention further provides a composition comprising an insulin dimer selected from the group consisting of Dimers 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, and 43.

The present invention further provides a method for treating diabetes comprising administering to an individual with diabetes a therapeutically effective amount of a composition comprising the insulin receptor partial agonist of any one of insulin dimers. In particular aspects the diabetes is Type 1 diabetes, Type 2 diabetes, or gestational diabetes.

The present invention further provides a composition for the treatment of diabetes comprising the any one of the above insulin dimers. In particular aspects the diabetes is Type 1 diabetes, Type 2 diabetes, or gestational diabetes.

The present invention further provides for the use of any one of the above the insulin dimers for the manufacture of a medicament for the treatment of diabetes. In particular aspects the diabetes is Type 1 diabetes, Type 2 diabetes, or gestational diabetes.

The present invention further provides a composition comprising any one of the aforementioned insulin dimers and a glucagon-like protein 1 (GLP-1) receptor agonist. In particular aspects, the GLP-1 agonist is liraglutide, dulaglutide, or albiglutide.

### BRIEF DESCRIPTION OF THE DRAWINGS

FIG. 1 shows the change in plasma glucose in diabetic minipigs over time for Dimers 4, 12, and 13 compared to recombinant human insulin (RHI). Dimers and RHI were administered at 0.69 nmol/kg.

FIG. 2 shows the change in plasma glucose in diabetic minipigs over time for Dimers 21, 24, and 35 compared to recombinant human insulin (RHI). Dimers and RHI were administered at 0.69 nmol/kg.

FIG. 3 shows the change in plasma glucose in diabetic minipigs over time for Dimers 36, 42, and 43 compared to recombinant human insulin (RHI). Dimers and RHI were administered at 0.69 nmol/kg.

### DETAILED DESCRIPTION OF THE INVENTION

The present invention provides compounds comprising two insulin molecules covalently linked to form a covalently-linked insulin dimer that may activate the insulin receptor with regular insulin-like potency and reduced maximum activity. These compounds are insulin receptor partial agonists (IRPA): they behave like other insulin analogs to lower glucose effectively but with lower risk of hypoglycemia.

### Definitions

Insulin—as used herein, the term means the active principle of the pancreas that affects the metabolism of carbohydrates in the animal body and which is of value in the treatment of diabetes mellitus. The term includes synthetic and biotechnologically derived products that are the same as, or similar to, naturally occurring insulins in structure, use, and intended effect and are of value in the treatment of diabetes mellitus. The term is a generic term that designates the 51 amino acid heterodimer comprising the A-chain peptide having the amino acid sequence shown in SEQ ID NO: 1 and the B-chain peptide having the amino acid sequence shown in SEQ ID NO: 2, wherein the cysteine residues a positions 6 and 11 of the A chain are linked in a disulfide bond, the cysteine residues at position 7 of the A chain and position 7 of the B chain are linked in a disulfide bond, and the cysteine residues at position 20 of the A chain and 19 of the B chain are linked in a disulfide bond.

Insulin analog or analogue—the term as used herein includes any heterodimer analogue or single-chain analogue that comprises one or more modification(s) of the native A-chain peptide and/or B-chain peptide. Modifications include but are not limited to substituting an amino acid for the native amino acid at a position selected from A4, A5, A8, A9, A10, A12, A13, A14, A15, A16, A17, A18, A19, A21, B1, B2, B3, B4, B5, B9, B10, B13, B14, B15, B16, B17, B18, B20, B21, B22, B23, B26, B27, B28, B29, and B30; deleting any or all of positions B1-4 and B26-30; or conjugating directly or by a polymeric or non-polymeric linker one or more acyl, polyethylglycine (PEG), or saccharide moiety (moieties); or any combination thereof. As exemplified by the N-linked glycosylated insulin analogues disclosed herein, the term further includes any insulin heterodimer and single-chain analogue that has been modified to have at least one N-linked glycosylation site and in particular, embodiments in which the N-linked glycosylation site is linked to or occupied by an N-glycan. Examples of insulin analogues include but are not limited to the heterodimer and single-chain analogues disclosed in published international application WO20100080606, WO2009/099763, and WO2010080609, the disclosures of which are incorporated herein by reference. Examples of single-chain insulin analogues also include but are not limited to those disclosed in published International Applications WO9634882, WO95516708, WO2005054291, WO2006097521, WO2007104734, WO2007104736, WO2007104737, WO2007104738, WO2007096332, WO2009132129; U.S. Pat. Nos. 5,304,473, 6,630,348 and 8,273,361; and Kristensen et al., Biochem. J. 305: 981-986 (1995), the disclosures of which are each incorporated herein by reference.

The term further includes single-chain and heterodimer polypeptide molecules that have little or no detectable activity at the insulin receptor but which have been modified to include one or more amino acid modifications or substitutions to have an activity at the insulin receptor that has at least 1%, 10%, 50%, 75%, or 90% of the activity at the insulin receptor as compared to native insulin and which further includes at least one N-linked glycosylation site. In particular aspects, the insulin analogue is a partial agonist that has less than 80% (or 70%) activity at the insulin receptor as does native insulin. These insulin analogues, which have reduced activity at the insulin growth hormone receptor and enhanced activity at the insulin receptor, include both heterodimers and single-chain analogues.

Single-chain insulin or single-chain insulin analog as used herein, the term encompasses a group of structurally-related proteins wherein the A-chain peptide or functional analogue and the B-chain peptide or functional analogue are covalently linked by a peptide or polypeptide of 2 to 35 amino acids or non-peptide polymeric or non-polymeric linker and which has at least 1%, 10%, 50%, 75%, or 90% of the activity of insulin at the insulin receptor as compared to native insulin. The single-chain insulin or insulin analogue further includes three disulfide bonds: the first disulfide bond is between the cysteine residues at positions 6 and 11 of the A-chain or functional analogue thereof, the second disulfide bond is between the cysteine residues at position 7 of the A-chain or functional analogue thereof and position 7 of the B-chain or functional analogue thereof, and the third disulfide bond is between the cysteine residues at position 20 of the A-chain or functional analogue thereof and position 19 of the B-chain or functional analogue thereof.

Insulin dimer—as used herein, the term refers to a dimer comprising two insulin molecules (also referred to as heterodimers) linked together via their respective lysine residues at or near the C-terminus of their respective B-chain polypeptides (i.e., the B29 Lysine) via a linking moiety as disclosed herein.

Boc—as used herein, the term refers to tert-butoxy carbonyl.

Heterocycles—as used herein, the term refers to a nonaromatic 3-10 membered monocyclic, 8-12 membered bicyclic, or 11-14 membered tricyclic ring system having 1-3 heteroatoms of monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heterocyclyl groups herein described may also contain fused rings. Fused rings are rings that share a common carbon-carbon bond or a common carbon atom (e.g., spiro-fused rings). Examples of heterocyclyl include, but are not limited to tetrahydrofuranyl, tetrahydropyranyl, piperidinyl, morpholino, pyrrolinyl and pyrrolidinyl.

Amino acid modification-as used herein, the term refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, WI), ChemPep Inc. (Miami, FL), and Genzyme Pharmaceuticals (Cambridge, MA). Atypical amino acids may be purchased from commercial suppliers, synthesized de novo, or chemically modified or derivatized from naturally occurring amino acids.

Amino acid substitution—as used herein refers to the replacement of one amino acid residue by a different amino acid residue.

Conservative amino acid substitution-as used herein, the term is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln, cysteic acid and homocysteic acid;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine
V. Large, aromatic residues:
   Phe, Tyr, Trp, acetyl phenylalanine

Treat—As used herein, the term “treat” (or “treating”, “treated”, “treatment”, etc.) refers to the administration of an IRPA of the present disclosure to a subject in need thereof with the purpose to alleviate, relieve, alter, ameliorate, improve or affect a condition (e.g., diabetes), a symptom or symptoms of a condition (e.g., hyperglycemia), or the predisposition toward a condition. For example, as used herein the term “treating diabetes” will refer in general to maintaining glucose blood levels near normal levels and may include increasing or decreasing blood glucose levels depending on a given situation.

The term AEG-C6 is depicted as:

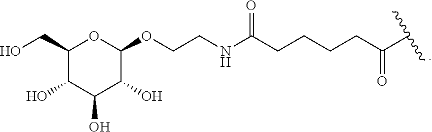 [SMILES: *C(=O)CCCCC(=O)NCCO[C@@H]1O[C@H](CO)[C@@H](O)[C@H](O)[C@H]1O]

Pharmaceutically acceptable carrier—as used herein, the term includes any of the standard pharmaceutical carriers, such as a phosphate buffered saline solution, water, emulsions such as an oil/water or water/oil emulsion, and various types of wetting agents suitable for administration to or by an individual in need. The term also encompasses any of the agents approved by a regulatory agency of the US Federal government or listed in the US Pharmacopeia for use in animals, including humans.

Pharmaceutically acceptable salt—as used herein, the term refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium, zinc, and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

Effective or therapeutically effective amount-as used herein refers to a nontoxic but sufficient amount of an insulin analog to provide the desired effect. For example one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” It is not always possible to determine the optimal effective amount prior to administration to or by an individual in need thereof. However, an appropriate “effective” amount in any individual case may be determined by one of ordinary skill in the art using routine experimentation.

Parenteral—as used herein, the term means not through the alimentary canal but by some other route such as intranasal, inhalation, subcutaneous, intramuscular, intraspinal, or intravenous.

The instant invention relates to insulin dimers where the level of insulin activity and partial agonist activity of the dimers is a function of the dimeric structure that involves the sequence of the insulin analog, the length of the dimerization linker, and the site of dimerization that connects the two insulin polypeptides. The insulin dimers of the present invention have reduced risk of promoting hypoglycemia when administered in high doses than native insulin or other insulin analogs when administered at high doses. The instant invention further relates to insulin dimers with capping group X introduced at B1 and/or B1′ positions of Formula I. As shown herein, the capping group improves chemical and biophysical stability of the insulin dimers while preserving their potency and biological profile as partial agonists of insulin receptors. Thus, the instant invention relates to insulin dimers with capping group X introduced at B1 and/or B1′ positions of Formula I that improve chemical and biophysical stability of the insulin dimers while preserving potency and biological profile.

The present invention provides partial agonist covalently-linked insulin dimers formulated as a novel and transformative basal insulin (once daily administration) that manifests improved therapeutic index (TI) over current standard of care (SOC) basal insulins. These molecules may lower glucose effectively with reduced risk of hypoglycemia in diabetic minipig and have the property of a once daily (QD) basal insulin. The improved TI may enable practitioners to more aggressively dose IRPA insulin dimer to achieve target goals for control of fasting glucose. Tight control of fasting glucose and HbA1c may allow these molecules to serve as 1) a stand-alone long-acting insulin with an enhanced efficacy and safety profile in Type 2 diabetes mellitus (T2DM) and 2) an improved foundational basal insulin in Type 1 diabetes mellitus (T1DM) (and some T2DM) for use with additional prandial rapid-acting insulin analogs (RAA) doses.

An ideal long-acting insulin provides continuous control of fasting glucose in diabetics with highly stable and reproducible PK/PD. However, currently available basal insulins, even those with improved stability and reproducibility of PK/PD continue to have a narrow therapeutic index and hypoglycemia incidents increase as glucose levels approach euglycemia target. This can often lead to underdosing to avoid hypoglycemia. Treatment with an IRPA of the present invention is expected to be safer with respect to hypoglycemia due to reduced maximal effect of the drug.

### Insulin A and B Chains

Disclosed herein are insulin or insulin analog dimers that have insulin receptor agonist activity. The level of insulin activity of the dimers is a function of the dimeric structure, the sequence of the insulin analog, the length of the dimerization linker, and the site of dimerization that connects the two insulin polypeptides. The insulin polypeptides of the present invention may comprise the native B and A chain sequences of human insulin (SEQ ID NOs: 1 and 2, respectively) or any of the known analogs or derivatives thereof that exhibit insulin agonist activity when linked to one another in a heteroduplex. Such analogs include, for example, proteins that having an A-chain and a B-chain that differ from the A-chain and B-chain of human insulin by having one or more amino acid deletions, one or more amino acid substitutions, and/or one or more amino acid insertions that do not destroy the insulin activity of the insulin analog.

One type of insulin analog, “monomeric insulin analog,” is well known in the art. These are fast-acting analogs of human insulin, including, for example, insulin analogs wherein:
(a) the amino acid residue at position B28 is substituted with Asp, Lys, Leu, Val, or Ala, and the amino acid residue at position B29 is Lys or Pro;
(b) the amino acid residues at any of positions B27 and B30 are deleted or substituted with a nonnative amino acid.
one embodiment an insulin analog is provided comprising an Asp substituted at position B28 (e.g., insulin aspart (NOVOLOG); see SEQ ID NO:9) or a Lys substituted at position 28 and a proline substituted at position B29 (e.g., insulin lispro (HUMALOG); see SEQ ID NO: 6). Additional monomeric insulin analogs are disclosed in Chance, et al., U.S. Pat. No. 5,514,646; Chance, et al., U.S. patent application Ser. No. 08/255,297; Brems, et al., Protein Engineering, 5:527-533 (1992); Brange, et al., EPO Publication No. 214,826 (published Mar. 18, 1987); and Brange, et al., Current Opinion in Structural Biology, 1:934-940 (1991). These disclosures are expressly incorporated herein by reference for describing monomeric insulin analogs.

Insulin analogs may also have replacements of the amidated amino acids with acidic forms. For example, Asn may be replaced with Asp or Glu. Likewise, Gln may be replaced with Asp or Glu. In particular, Asn(A18), Asn(A21), or Asp(B3), or any combination of those residues, may be replaced by Asp or Glu. Also, Gln(A15) or Gln(B4), or both, may be replaced by either Asp or Glu.

As disclosed herein insulin single chain analogs are provided comprising a B chain and A chain of human insulin, or analogs or derivative thereof, wherein the carboxy terminus of the B chain is linked to the amino terminus of the A chain via a linking moiety. In one embodiment the A chain is amino acid sequence GIVEQCCTSICSLYQLENYCN (SEQ ID NO: 1 and the B chain comprises amino acid sequence FVNQHLCGSH LVEALYLVCGERGFFYTPKT (SEQ ID NO: 2) or a carboxy shortened sequence thereof having B30 deleted, and analogs of those sequences wherein each sequence is modified to comprise one to five amino acid substitutions at positions corresponding to native insulin positions selected from A5, A8, A9, A10, A14, A15, A17, A18, A21, B1, B2, B3, B4, B5, B9, B10, B13, B14, B20, B22, B23, B26, B27, B28, B29 and B30, with the proviso that at least one of B28 or B29 is lysine. In one embodiment the amino acid substitutions are conservative amino acid substitutions. Suitable amino acid substitutions at these positions that do not adversely impact insulin's desired activities are known to those skilled in the art, as demonstrated, for example, in Mayer, et al., Insulin Structure and Function, Biopolymers. 2007;88(5):687-713, the disclosure of which is incorporated herein by reference.

In accordance with one embodiment, the insulin analog peptides may comprise an insulin A chain and an insulin B chain or analogs thereof, wherein the A chain comprises an amino acid sequence that shares at least 70% sequence identity (e.g., 70%, 75%, 80%, 85%, 90%, 95%) over the length of the native peptide, with GIVEQCCTSICSLYQLENYCN (SEQ ID NO: 1) and the B chain comprises an amino acid sequence that shares at least 60% sequence identity (e.g., 60%, 65%, 70%, 75%, 80%, 85%, 90%, 95%) over the length of the native peptide, with FVNQHLCGSHLVEALYLVCGERGFFYTPKT (SEQ ID NO: 2) or a carboxy shortened sequence thereof having B30 deleted.

Additional amino acid sequences can be added to the amino terminus of the B chain or to the carboxy terminus of the A chain of the insulin polypeptides of the present invention. For example, a series of negatively charged amino acids can be added to the amino terminus of the B chain, including for example a peptide of 1 to 12, 1 to 10, 1 to 8 or 1 to 6 amino acids in length and comprising one or more negatively charged amino acids including for example glutamic acid and aspartic acid. In one embodiment the B chain amino terminal extension comprises 1 to 6 charged amino acids. In accordance with one embodiment, the insulin polypeptides disclosed comprise a C-terminal amide or ester in place of a C-terminal carboxy late on the A chain.

In various embodiments, the insulin analog has an isoelectric point that has been shifted relative to human insulin. In some embodiments, the shift in isoelectric point is achieved by adding one or more arginine, lysine, or histidine residues to the N-terminus of the insulin A-chain peptide and/or the C-terminus of the insulin B-chain peptide. Examples of such insulin polypeptides include Arg^{A0}-human insulin, Arg^{B31}Arg^{B32}-human insulin, Gly^{A21}Arg^{B31}Arg^{B32}-human insulin, ArgA0Arg^{B31}Arg^{B32}-human insulin, and Arg^{A0}Gly^{A21}Arg^{B31}Arg^{B32}-human insulin. By way of further example, insulin glargine (LANTUS; see SEQ ID NOs: 7 and 8) is an exemplary long-acting insulin analog in which Asn^{A21} has been replaced by glycine, and two arginine residues have been covalently linked to the C-terminus of the B-peptide. The effect of these amino acid changes was to shift the isoelectric point of the molecule, thereby producing a molecule that is soluble at acidic pH (e.g., pH 4 to 6.5) but insoluble at physiological pH. When a solution of insulin glargine is injected into the muscle, the pH of the solution is neutralized and the insulin glargine forms microprecipitates that slowly release the insulin glargine over the 24 hour period following injection with no pronounced insulin peak and thus a reduced risk of inducing hypoglycemia. This profile allows a once-daily dosing to provide a patient's basal insulin. Thus, in some embodiments, the insulin analog comprises an A-chain peptide wherein the amino acid at position A21 is glycine and a B-chain peptide wherein the amino acids at position B31 and B32 are arginine. The present disclosure encompasses all single and multiple combinations of these mutations and any other mutations that are described herein (e.g., Gly^{A21}-human insulin, Gly^{A21}Arg^{B31}-human insulin, Arg^{B31}Arg^{B32}-human insulin, Arg^{B31}-human insulin).

In particular aspects of the insulin receptor partial agonists, one or more amidated amino acids of the insulin analog are replaced with an acidic amino acid, or another amino acid. For example, asparagine may be replaced with aspartic acid or glutamic acid, or another residue. Likewise, glutamine may be replaced with aspartic acid or glutamic acid, or another residue. In particular, Asn^{A18}, Asn^{A21}, or Asn^{B3}, or any combination of those residues, may be replaced by aspartic acid or glutamic acid, or another residue. Gln^{A15} or Gln^{B4}, or both, may be replaced by aspartic acid or glutamic acid, or another residue. In particular aspects of the insulin receptor partial agonists, the insulin analogs have an aspartic acid, or another residue, at position A21 or aspartic acid, or another residue, at position B3, or both.

One skilled in the art will recognize that it is possible to replace yet other amino acids in the insulin analog with other amino acids while retaining biological activity of the molecule. For example, without limitation, the following modifications are also widely accepted in the art: replacement of the histidine residue of position B10 with aspartic acid (His^{B10} to Asp^{B10}); replacement of the phenylalanine residue at position B1 with aspartic acid (PheB1 to AspB1); replacement of the threonine residue at position B30 with alanine (ThrB30 toAlaB30); replacement of the tyrosine residue at position B26 with alanine (TyrB26 to AlaB26); and replacement of the serine residue at position B9 with aspartic acid (SerB9 to AspB9).

In various embodiments, the insulin analog has a protracted profile of action. Thus, in certain embodiments, the insulin analog may be acylated with a fatty acid. That is, an amide bond is formed between an amino group on the insulin analog and the carboxylic acid group of the fatty acid. The amino group may be the alpha-amino group of an N-terminal amino acid of the insulin analog, or may be the epsilon-amino group of a lysine residue of the insulin analog.

The insulin analog may be acylated at one or more of the three amino groups that are present in wild-type human insulin may be acylated on lysine residue that has been introduced into the wild-type human insulin sequence. In particular aspects of the insulin receptor partial agonists, the insulin analog may be acylated at position B1, B1′, or both B1 and B1′.

Examples of insulin analogs can be found for example in published International Application WO9634882, WO95516708; WO20100080606, WO2009/099763, and WO2010080609, U.S. Pat. No. 6,630,348, and Kristensen et al., Biochem. J. 305: 981-986 (1995), the disclosures of which are incorporated herein by reference). In further embodiments, the in vitro glycosylated or in vivo N-glycosylated insulin analogs may be acylated and/or pegylated.

In particular aspects of the insulin dimer, each A-chain polypeptide independently comprises the amino acid sequenceGX₂X₃EQCCX₈SICSLYQLX₁₇NX₁₉CX₂₃ (SEQ ID NO: 3) and each B-chain polypeptide independently comprises the amino acid sequence X₂₅LCGX₂₉X₃₀LVEALYLVCGERGFX₂₇YTX₃₁X₃₂ (SEQ ID NO:4) or X₂₂VNQX₂₅X₂₆CGX₂₉X₃₀LVEALYLVCGERGFX₂₇YTX₃₁X₃₂X₃₃X₃₄X₃₅ (SEQ ID NO: 5) wherein X₂ is isoleucine or threonine; X₃ is valine, glycine, or leucine; X₈ is threonine or histidine; X₁₇ is glutamic acid or glutamine; X₁₉ is tyrosine, 4-methoxy-phenylalanine, alanine, or 4-amino phenylalanine; X₂₃ is asparagine or glycine; X₂₂ is or phenylalanine and desamino-phenylalanine; X₂₅ is histidine or threonine; X₂₆ is leucine or glycine; X₂₇ is phenylalanine or aspartic acid; X₂₉ is alanine, glycine, or serine; X₃₀ is histidine, aspartic acid, glutamic acid, homocysteic acid, or cysteic acid; X₃₁ is aspartic acid, proline, or lysine; X₃₂ is lysine or proline; X₃₃ is threonine, alanine, or absent; X₃₄ is arginine or absent; and X₃₅ is arginine or absent; with the proviso at least one of X₃₁ or X₃₂ is lysine.

### Linking Moiety

The insulin dimers disclosed herein are formed between a first and second insulin polypeptide wherein each insulin polypeptide comprises an A chain and a B chain. The first and second insulin polypeptides may be two chain insulin analogs (i.e., wherein the A and B chains are linked only via inter-chain disulfide bonds between internal cysteine residues) wherein the first and second insulin polypeptides are linked to one another to form the dimer by a covalent bond, bifunctional linker, or other means known in the art to link linking moieties on the respective B chains. In accordance with one embodiment the first and second insulin polypeptides are linked to one another by a bifunctional linker joining the side chain of the B29 lysine of the B chain of the first insulin polypeptide to the side chain of the B29 amino acid of the B′ chain of the second insulin polypeptide.

The following Table I shows exemplary linker structures, which may be used to construct the dimers of the present invention. The linker reagent shown comprise 2,5-dioxopyrrolidin-1yl groups for conjugating to the epsilon amino group of the B29 lysine. Also shown are exemplary linking moieties of the invention.

TABLE I

| | | | Linking Moiety (Wavy |
|---|---|---|---|
| | | | lines indicate |
| Lin- | Linking | | attachment to B29 and |
| ker | Reagent | Linking Reagent | B29′ sites of two |
| No. | Name | Structure | insulins) |
| 1 | bis(2,5- dioxopyrrolidin- 1-yl) 4,7,10,13- tetraoxahexa- decanedioate | 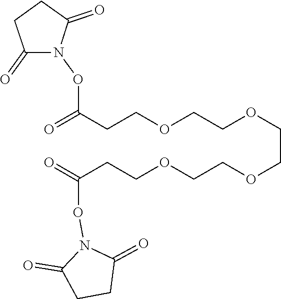 [SMILES: O=C(CCOCCOCCOCCOCCC(=O)ON1C(=O)CCC1=O)ON1C(=O)CCC1=O] | 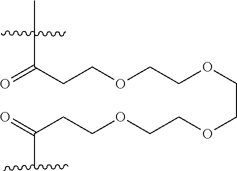 [SMILES: *C(=O)CCOCCOCCOCCOCCC(*)=O] |
| 2 | bis(2,5- dioxopyrrolidin- 1-yl) 4,7,10,13,16- pentaoxanona- decanedioate | 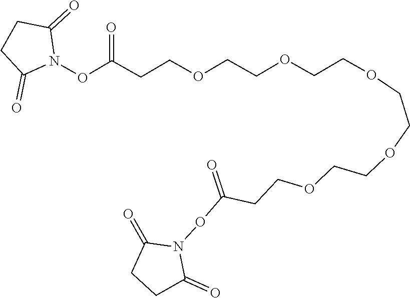 [SMILES: O=C(CCOCCOCCOCCOCCOCCC(=O)ON1C(=O)CCC1=O)ON1C(=O)CCC1=O] | 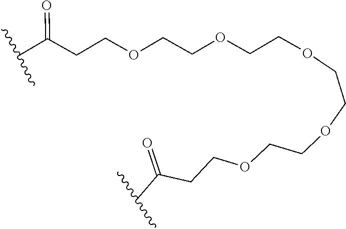 [SMILES: *C(=O)CCOCCOCCOCCOCCOCCC(*)=O] |
| 3 | bis(2,5- dioxopyrrolidin- 1-yl) 4,7,10,13,16,19- hexaoxadoco- sanedioate | 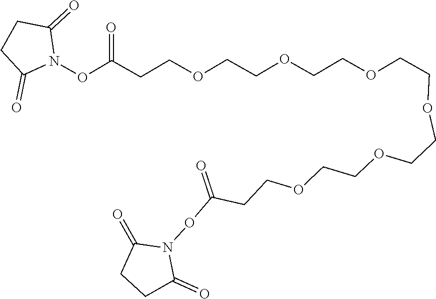 [SMILES: O=C(CCOCCOCCOCCOCCOCCOCCC(=O)ON1C(=O)CCC1=O)ON1C(=O)CCC1=O] | 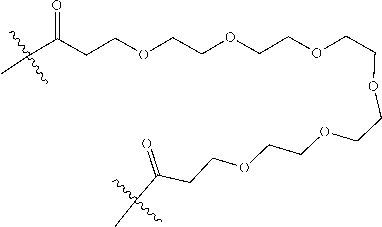 [SMILES: *C(=O)CCOCCOCCOCCOCCOCCOCCC(*)=O] |
| 4 | bis(2,5- dioxopyrrolidin- 1-yl) 4,7,10,13, 16,19,22- heptaoxapenta- cosanedioate | 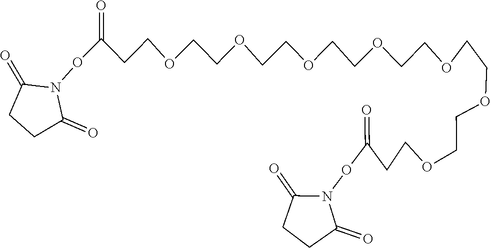 [SMILES: O=C(CCOCCOCCOCCOCCOCCOCCOCCC(=O)ON1C(=O)CCC1=O)ON1C(=O)CCC1=O] | 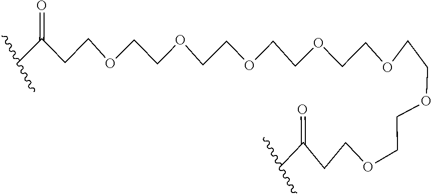 [SMILES: *C(=O)CCOCCOCCOCCOCCOCCOCCOCCC(*)=O] |
| 5 | bis(2,5- dioxopyrrolidin- 1-yl) 4,7,10,13,16, 19,22,25,28- nonaoxahen- triacontane dioate | 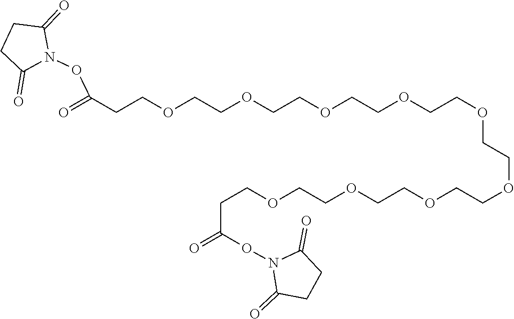 [SMILES: O=C(CCOCCOCCOCCOCCOCCOCCOCCOCCOCCC(=O)ON1C(=O)CCC1=O)ON1C(=O)CCC1=O] | 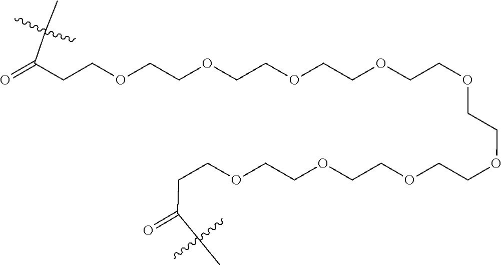 [SMILES: *C(=O)CCOCCOCCOCCOCCOCCOCCOCCOCCOCCC(*)=O] |
| 6 | bis(2,5- dioxopyrrolidin- 1-yl) 4,7,10,13, 16,19,22, 25,28,31,34- undecaoxahepta- triacontanedioate | 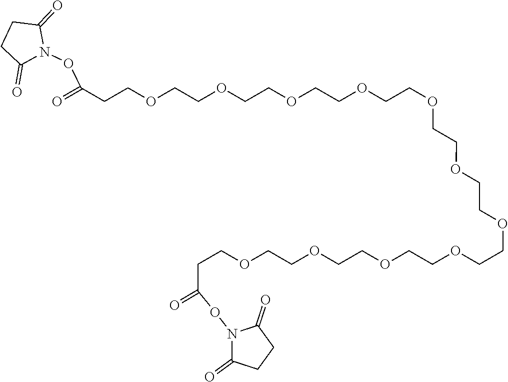 [SMILES: O=C(CCOCCOCCOCCOCCOCCOCCOCCOCCOCCOCCOCCC(=O)ON1C(=O)CCC1=O)ON1C(=O)CCC1=O] | 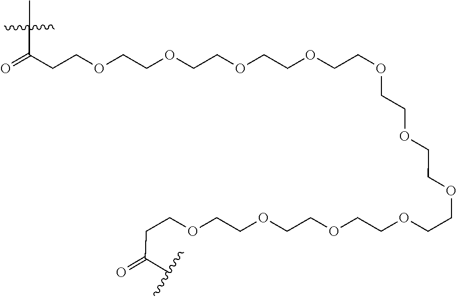 [SMILES: *C(=O)CCOCCOCCOCCOCCOCCOCCOCCOCCOCCOCCOCCC(*)=O] |
| 7 | bis(2,5- dioxopyrrolidin- 1-yl) 4,7,10,13,16, 19,22,25, 28,31,34,37,40- trideca- oxatritetra- contanedioate | 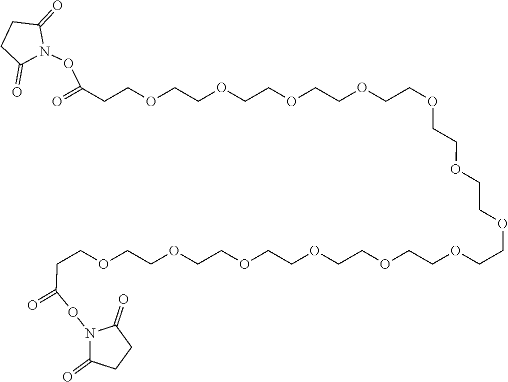 [SMILES: O=C(CCOCCOCCOCCOCCOCCOCCOCCOCCOCCOCCOCCOCCOCCC(=O)ON1C(=O)CCC1=O)ON1C(=O)CCC1=O] | 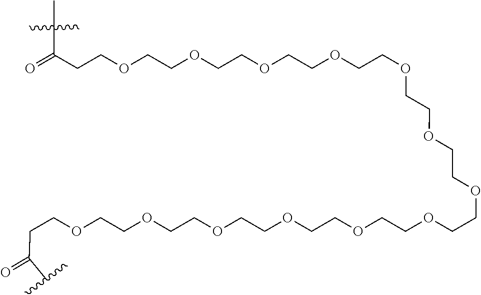 [SMILES: *C(=O)CCOCCOCCOCCOCCOCCOCCOCCOCCOCCOCCOCCOCCOCCC(*)=O] |
| 8 | bis(2,5- dioxopyrrolidin- 1-yl) 4,7,10,13, 16,19,22,25, 28,31,34, 37,40,43,46, 49,52- heptadeca- oxapenta- pentacontane- dioate | 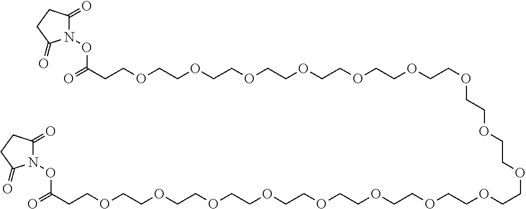 [SMILES: O=C(CCOCCOCCOCCOCCOCCOCCOCCOCCOCCOCCOCCOCCOCCOCCOCCOCCOCCC(=O)ON1C(=O)CCC1=O)ON1C(=O)CCC1=O] | 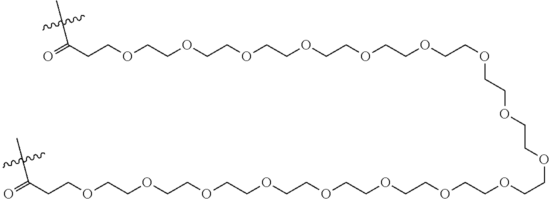 [SMILES: *C(=O)CCOCCOCCOCCOCCOCCOCCOCCOCCOCCOCCOCCOCCOCCOCCOCCOCCOCCC(*)=O] |
| 9 | bis(2,5- dioxopyrrolidin- 1-yl) octanedioate | 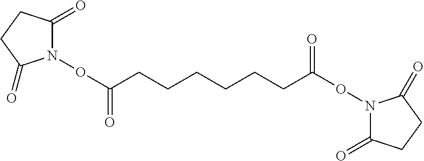 [SMILES: O=C(CCCCCCC(=O)ON1C(=O)CCC1=O)ON1C(=O)CCC1=O] | 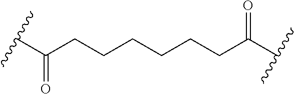 [SMILES: *C(=O)CCCCCCC(*)=O] |
| 10 | bis(2,5- dioxopyrrolidin- 1-yl) octadecanedioate | 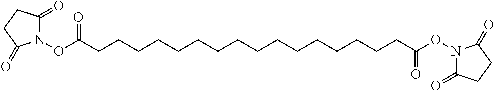 [SMILES: O=C(CCCCCCCCCCCCCCCCC(=O)ON1C(=O)CCC1=O)ON1C(=O)CCC1=O] | 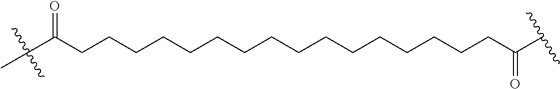 [SMILES: *C(=O)CCCCCCCCCCCCCCCCC(*)=O] |
| 11 | bis(2,5- dioxopyrrolidin- 1-yl) icosanedioate | 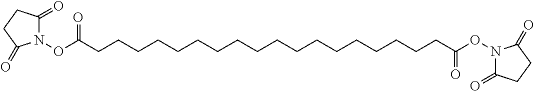 [SMILES: O=C(CCCCCCCCCCCCCCCCCCC(=O)ON1C(=O)CCC1=O)ON1C(=O)CCC1=O] | 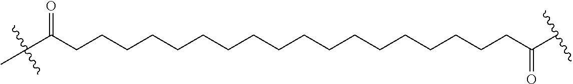 [SMILES: *C(=O)CCCCCCCCCCCCCCCCCCC(*)=O] |
| 12 | bis(2,5- dioxopyrrolidin- 1-yl) (2R,5S)-1,4- dioxane-2,5- dicarboxylate | 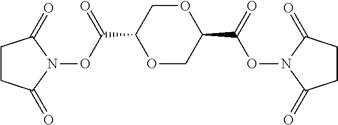 [SMILES: O=C(ON1C(=O)CCC1=O)[C@@H]1CO[C@@H](C(=O)ON2C(=O)CCC2=O)CO1] | 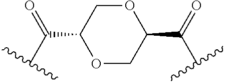 [SMILES: *C(=O)[C@@H]1CO[C@@H](C(*)=O)CO1] |
| 13 | bis(2,5- dioxopyrrolidin- 1-yl) (1r,4r)- cyclohexane-1,4- dicarboxylate | 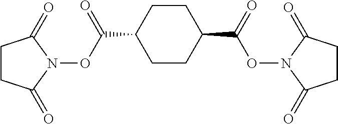 [SMILES: O=C1CCC(=O)N1OC(=O)[C@H]1CC[C@H](C(=O)ON2C(=O)CCC2=O)CC1] | 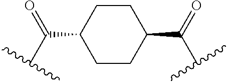 [SMILES: *C(=O)[C@H]1CC[C@H](C(*)=O)CC1] |
| 14 | 2,5- dioxopyrrolidin- 1-yl 4-((2-(2- (3-((2,5- dioxopyrrolidin- 1-yl)oxy)-3- oxopropoxy) ethoxy) | 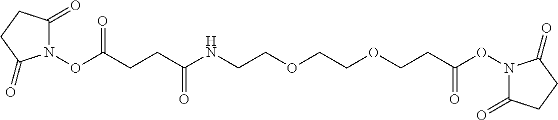 [SMILES: O=C(CCC(=O)ON1C(=O)CCC1=O)NCCOCCOCCC(=O)ON1C(=O)CCC1=O] | 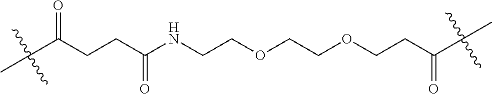 [SMILES: *C(=O)CCOCCOCCNC(=O)CCC(*)=O] |
| | ethyl)amino)-4- | | |
| | oxobutanoate | | |
| 15 | bis(2,5- dioxopyrrolidin- 1-yl) 8,14-dioxo- 4,11,18-trioxa- 7,15- | 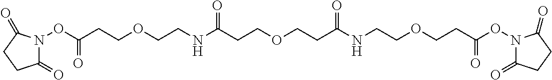 [SMILES: O=C(CCOCCC(=O)NCCOCCC(=O)ON1C(=O)CCC1=O)NCCOCCC(=O)ON1C(=O)CCC1=O] | 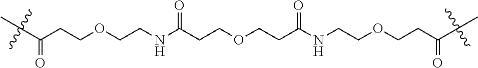 [SMILES: *C(=O)CCOCCNC(=O)CCOCCC(=O)NCCOCCC(*)=O] |
| | diazahenico- | | |
| | sanedioate | | |
| 16 | 2,5- dioxopyrrolidin- 1-yl 3-(2-(3- ((2-((2,5- dioxopyrrolidin- 1-yl)oxy)-2- oxoethyl) | 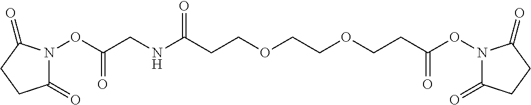 [SMILES: O=C(CCOCCOCCC(=O)ON1C(=O)CCC1=O)NCC(=O)ON1C(=O)CCC1=O] | 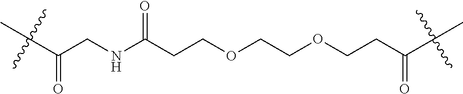 [SMILES: *C(=O)CCOCCOCCC(=O)NCC(*)=O] |
| | amino)-3- | | |
| | oxopropoxy) | | |
| | ethoxy) | | |
| | propanoate | | |

In one embodiment, the linking moiety comprises a PEG linker, a short linear polymer of about 2-25 ethylene glycol units or 2, 3, 4, 5, 6, 7, 8, 9, 10, 11, 12, 13, 14, 15, 16, or 25 ethylene glycol units and optionally one or more amino acids. In particular aspects of the insulin receptor partial agonists, the PEG linker comprises the structure (PEG)₂, (PEG)₃, (PEG)₄, (PEG)₅, (PEG)₆, (PEG)₇, (PEG)₈, (PEG)₉, (PEG)₁₀, (PEG)₁₁, (PEG)₁₂, (PEG)₁₃, (PEG)₁₄, (PEG)₁₅, (PEG)₁₆, (PEG)₁₇, or (PEG)₂₅. The PEG linker may be a bifunctional linker that may be covalently conjugated or linked to epsilon amino group of the position B29 lysine residues of the first and second insulin polypeptides. The structure of a bifunctional PEG linker conjugated to the epsilon amino group of the lysine groups at position B29 of the first and second insulin polypeptides may be represented by the following general formula

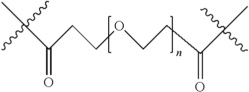

wherein n=1, 2, 3, 4, 5, 6, 7, 8, 9, 10, 11, 12, 13, 14, 15, 16, 17, or 25 and the wavy line indicates the bond between the linker and the epsilon amino group. Methods for conjugating PEG to the epsilon amino group of lysine are well known in the art, see for example, Veronese, Biomaterials 22: 405-417 (2001).

In particular aspects of the insulin receptor partial agonists, PEG linking moiety conjugating the epsilon amino group of the lysine at position B29 of the first insulin polypeptide to the epsilon amino acid of the lysine at position B29 of the second insulin polypeptide is

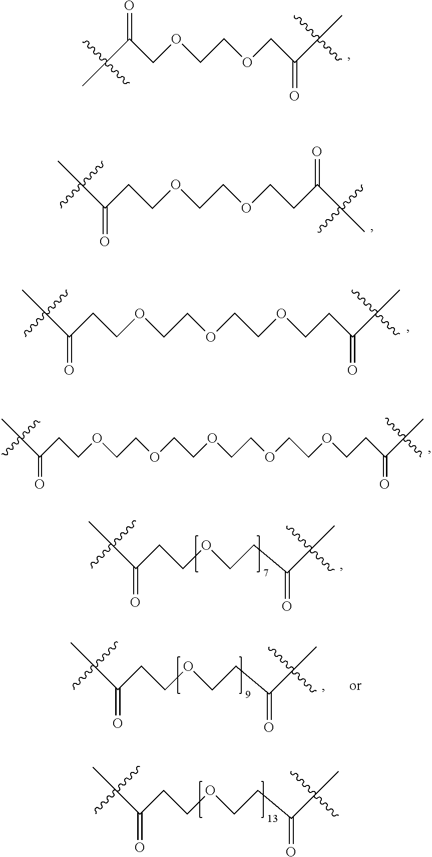

wherein the wavy lines indicate the bond between the linker and the epsilon amino group of the lysine at position B29 of the insulin polypeptides.

In another embodiment, the linking moiety comprises an acyl moiety comprising 4, 5, 6, 7, 8, 9, 10, 11, 12, 13, 14 15, or 16 carbons. In particular aspects of the insulin receptor partial agonists, the acyl moiety is a succinyl (4), adipoyl (C6), suberyol (C8), or hexadecanedioyl (C16) moiety. The acyl moiety may comprise a bifunctional linker that may be covalently conjugated or linked to epsilon amino group of the position B29 lysine residues of the first and second insulin polypeptides. The structure of a bifunctional acyl linker conjugated to the epsilon amino group of the lysine group at position B29 of the first and second insulin polypeptides may be represented by the following general formula

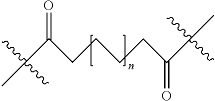

wherein n=0, 1, 2, 3, 4, 5, 6, 7, 8, 9, 10, 11, or 12 and the wavy lines indicate the bond between the linker and the epsilon amino group of the lysine at position B29 of the insulin polypeptides.

In particular aspects of the insulin receptor partial agonists, acyl linking moiety conjugating the epsilon amino group of the lysine at position B29 of the first insulin polypeptide to the epsilon amino acid of the lysine at position B29 of the second insulin polypeptide is

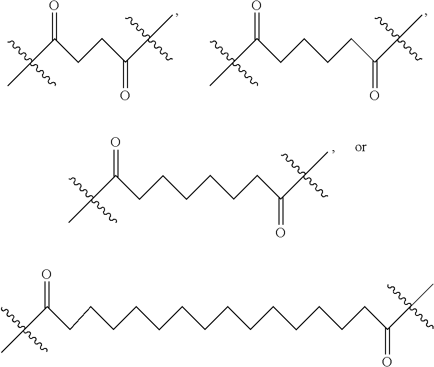 [SMILES: *C(=O)CCC(*)=O ; *C(=O)CCCCC(*)=O ; *C(=O)CCCCCCC(*)=O ; *C(=O)CCCCCCCCCCCCCCC(*)=O]

wherein the wavy lines indicate the bond between the linker and the epsilon amino group of the lysine at position B29 of the insulin polypeptides.

Conjugation of a bifunctional linker to the epsilon amino group of the lysine residue at position B29 of the B-chain polypeptide of two insulin or insulin analog molecules to form the insulin dimer linked by a linking moiety may be schematically shown as

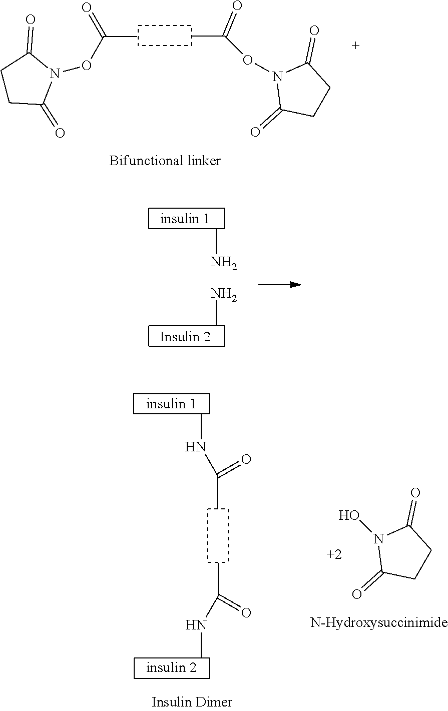

wherein the insulin 1 and insulin 2 molecules may be the same or different and the bifunctional linker and resulting linking moiety following conjugation may have the structure of any linker and resulting linking moiety disclosed herein.

### Modification of Insulin Polypeptides

In some embodiments, at least one of the B-chain polypeptides of the insulin receptor partial agonist is modified to comprise an acyl group. The acyl group can be covalently linked directly to an amino acid of the insulin polypeptide, or indirectly to an amino acid of the insulin polypeptide via a spacer, wherein the spacer is positioned between the amino acid of the insulin polypeptide and the acyl group. example, acylation may occur at any position including any amino acid of the B-chain polypeptides as well as a position within the linking moiety, provided that the activity exhibited by the non-acylated insulin polypeptide is retained upon acylation. Non-limiting examples include acylation at position B1 of the B chain.

In one specific aspect of the invention, the first and/or second insulin polypeptide (or derivative or conjugate thereof) is modified to comprise an acyl group by direct acylation of an amine, hydroxyl, or thiol of a side chain of an amino acid of the insulin polypeptide. In some embodiments, the first and/or second insulin polypeptide is directly acylated through the side chain amine, hydroxyl, or thiol of an amino acid. In this regard, an insulin polypeptide may be provided that has been modified by one or more amino acid substitutions in the B-chain polypeptide sequence, including for example at positions B1, B10, or B22 or at any position of the linking moiety with an amino acid comprising a side chain amine, hydroxyl, or thiol. An example of a spacer as illustrated in Scheme X below.

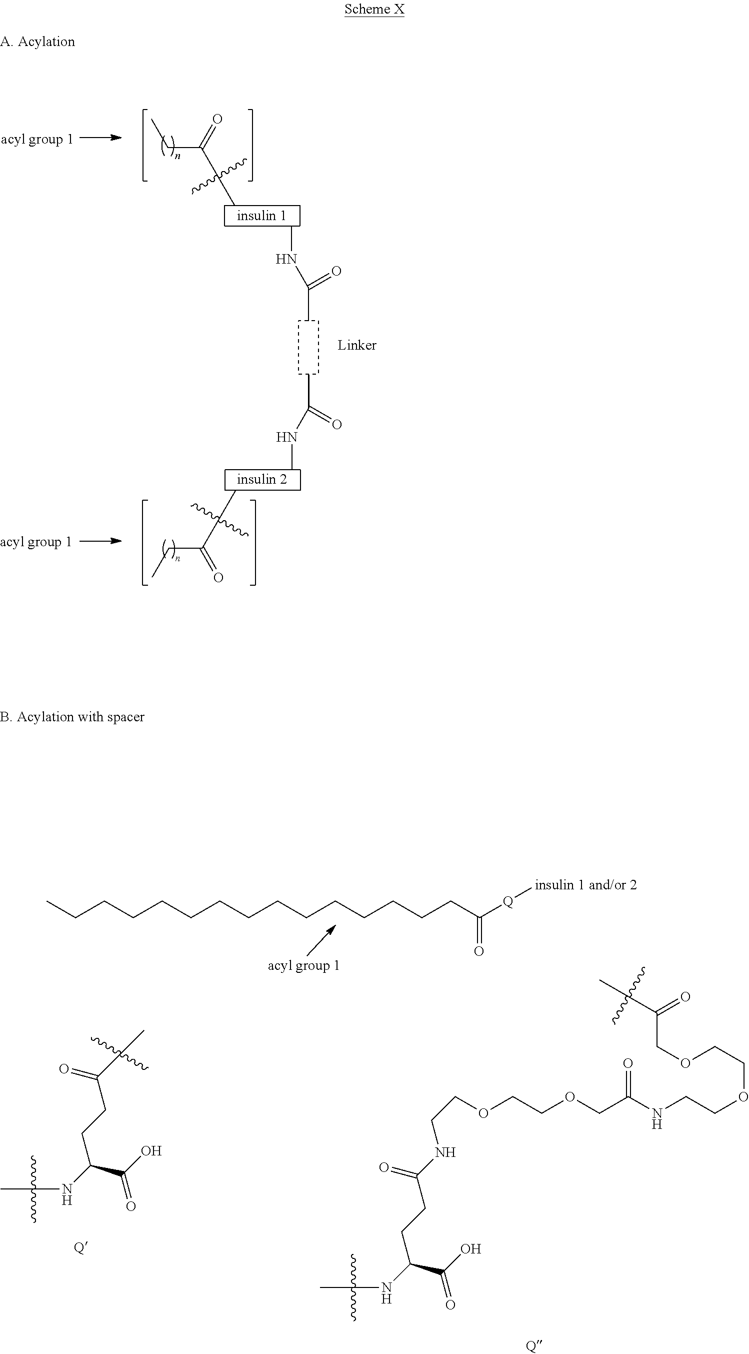

wherein in A. the wavy line illustrates point of attachment to side chain of insulin peptide and in B. the wavy line at the amino group of the spacer illustrates point of attachment to the acyl group 1 and the wavy line at the carbonyl group of the spacer illustrates point of attachment to the insulin peptide, Q is a spacer represented, for example, as Q′ and Q″ and n is a C₁₅ alkyl.chain.

In some embodiments, the spacer between the first and/or second insulin polypeptide and the acyl group is an amino acid comprising a side chain amine, hydroxyl, or thiol (or a dipeptide or tripeptide comprising an amino acid comprising a side chain amine, hydroxyl, or thiol). In some embodiments, the spacer comprises a hydrophilic bifunctional spacer. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, KY). In one embodiment, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate.

In some embodiments, the spacer between the first and/or second insulin polypeptide and the acyl group is a hydrophobic bifunctional spacer. Hydrophobic bifunctional spacers are known in the art. See, e.g., Bioconjugate Techniques, G. T. Hermanson (Academic Press, San Diego, CA, 1996), which is incorporated by reference in its entirety. In certain embodiments, the hydrophobic bifunctional spacer comprises two or more reactive groups, e.g., an amine, a hydroxyl, a thiol, and a carboxyl group or any combinations thereof. In certain embodiments, the hydrophobic bifunctional spacer comprises a hydroxyl group and a carboxylate. In other embodiments, the hydrophobic bifunctional spacer comprises an amine group and a carboxylate. In other embodiments, the hydrophobic bifunctional spacer comprises a thiol group and a carboxylate. Suitable hydrophobic bifunctional spacers comprising a carboxylate and a hydroxyl group or a thiol group are known in the art and include, for example, 8-hydroxyoctanoic acid and 8-mercaptooctanoic acid.

In accordance with certain embodiments the bifunctional spacer can be a synthetic or naturally occurring amino acid comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. Each amino acid of the dipeptide or tripeptide spacer attached to the insulin polypeptide can be independently selected from the group consisting of: naturally-occurring and/or non-naturally occurring amino acids, including. for example. any of the D or L isomers of the naturally-occurring amino acids (Ala, Cys, Asp, Glu, Phe, Gly, His, Ile, Lys, Leu, Met, Asn, Pro, Arg, Ser, Thr, Val, Trp, Tyr), or any D or L isomers of the non-naturally occurring amino acids selected from the group consisting of: β-alanine (β-Ala), N-α-methyl-alanine (Me-Ala), aminobutyric acid (Abu), α-aminobutyric acid (γ-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl) alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ-Glu), homoserine (Hse), hydroxyproline (Hyp), isoleucine N-methoxy-N-methyl amide, methyl-isoleucine (MeIle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met(O2)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-Cl)), 4-fluorophenylalanine (Phe(4-F)), 4-nitrophenylalanine (Phe(4-NO2)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), U-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4,-tetrahydro-isoquinoline-3-carboxylic acid (Tic), tetrahydropyranglycine, thienylalanine (Thi), U-Benzyl-phosphotyrosine, O-Phosphotyrosine, methoxytyrosine, ethoxytyrosine, O-(bis-dimethylamino-phosphono)-tyrosine, tyrosine sulfate tetrabutylamine, methyl-valine (MeVal), 1-amino-1-cyclohexane carboxylic acid (Acx), aminovaleric acid, beta-cyclopropyl-alanine (Cpa), propargylglycine (Prg), allylglycine (Alg), 2-amino-2-cyclohexyl-propanoic acid (2-Cha), tertbutylglycine (Tbg), vinylglycine (Vg), 1-amino-1-cyclopropane carboxylic acid (Acp), 1-amino-1-cyclopentane carboxylic acid (Acpe), alkylated 3-mercaptopropionic acid, 1-amino-1-cyclobutane carboxylic acid (Acb). In some embodiments the dipeptide spacer is selected from the group consisting of: Ala-Ala, β-Ala-β-Ala, Leu-Leu, Pro-Pro, γ-aminobutyric acid-γ-aminobutyric acid, and γ-Glu-γ-Glu.

The first and/or second insulin polypeptide may be modified to comprise an acyl group by acylation of a long chain alkane. In specific aspects, the long chain alkane comprises an amine, hydroxyl, or thiol group (e.g. octadecylamine, tetradecanol, and hexadecanethiol) which reacts with a carboxyl group, or activated form thereof, of the insulin polypeptide. The carboxyl group, or activated form thereof, of the insulin polypeptide can be part of a side chain of an amino acid (e.g., glutamic acid, aspartic acid) of the insulin polypeptide or can be part of the peptide backbone.

In certain embodiments, the first and/or second insulin polypeptide is modified to comprise an acyl group by acy lation of the long chain alkane by a spacer which is attached to the insulin polypeptide. In specific aspects, the long chain alkane comprises an amine, hydroxyl, or thiol group which reacts with a carboxyl group, or activated form thereof, of the spacer. Suitable spacers comprising a carboxyl group, or activated form thereof, are described herein and include, for example, bifunctional spacers, e.g., amino acids, dipeptides, tripeptides, hydrophilic bifunctional spacers and hydrophobic bifunctional spacers. As used herein, the term “activated form of a carboxyl group” refers to a carboxyl group with the general formula R(C═O)X, wherein X is a leaving group and R is the insulin polypeptide or the spacer. For example, activated forms of a carboxyl groups may include, but are not limited to, acyl chlorides, anhydrides, and esters. In some embodiments, the activated carboxyl group is an ester with an N-hydroxysuccinimide (NHS) leaving group.

With regard to these aspects of the invention, in which a long chain alkane is acylated by the peptide, the insulin polypeptide or the spacer, the long chain alkane may be of any size and can comprise any length of carbon chain. The long chain alkane can be linear or branched. In certain aspects, the long chain alkane is a C₄ to C₃₀ alkane. For example, the long chain alkane can be any of a C₄ alkane, C₆ alkane, C₈ alkane, C₁₀ alkane, C₁₂ alkane, C₁₄ alkane, C₁₆ alkane, C₁₈ alkane, C₂₀ alkane, C₂₂ alkane, C₂₄ alkane, C₂₆ alkane, C₂₈ alkane, or a C₃₀ alkane. In some embodiments, the long chain alkane comprises a C₈ to C₂₀ alkane, e.g., a C₁₄ alkane, C₁₆ alkane, or a C₁₈ alkane.

In some embodiments, an amine, hydroxyl, or thiol group of the first and/or second insulin polypeptide is acylated with a cholesterol acid. In a specific embodiment, the peptide is linked to the cholesterol acid through an alkylated des-amino Cys spacer, i.e., an alkylated 3-mercaptopropionic acid spacer. Suitable methods of peptide acylation via amines, hydroxyls, and thiols are known in the art. See, for example, Miller, Biochem Biophys Res Commun 218: 377-382 (1996); Shimohigashi and Stammer, Int J Pept Protein Res 19: 54-62 (1982); and Previero et al., Biochim Biophys Acta 263: 7-13 (1972) (for methods of acylating through a hydroxyl); and San and Silvius, J Pept Res 66: 169-180 (2005) (for methods of acylating through a thiol); Bioconjugate Chem. “Chemical Modifications of Proteins: History and Applications” pages 1, 2-12 (1990); Hashimoto et al., Pharmacuetical Res. “Synthesis of Palmitoyl Derivatives of Insulin and their Biological Activity” Vol. 6, No: 2 pp. 171-176 (1989).

The acyl group of the acylated peptide the first and/or second insulin polypeptide can be of any size, e.g., any length carbon chain, and can be linear or branched. In some specific embodiments of the invention, the acyl group is a C₄ to C₃₀ fatty acid. For example, the acyl group can be any of a C₄ fatty acid, C₆ fatty acid, C₈ fatty acid, C₁₀ fatty acid, C₁₂ fatty acid, C₁₄ fatty acid, C₁₆ fatty acid, C₁₈ fatty acid, C₂₀ fatty acid, C₂₂ fatty acid, C₂₄ fatty acid, C₂₆ fatty acid, C₂₈ fatty acid, or a C₃₀ fatty acid. In some embodiments, the acyl group is a C₈ to C₂₀ fatty acid, e.g., a C₁₄ fatty acid or a C₁₆ fatty acid. In some embodiments, the acyl group is carbamoyl.

In an alternative embodiment, the acyl group is a bile acid. The bile acid can be any suitable bile acid, including, but not limited to, cholic acid, chenodeoxycholic acid, deoxycholic acid, lithocholic acid, taurocholic acid, glycocholic acid, and cholesterol acid.

The acy lated first and/or second insulin polypeptide described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein. In some embodiments the acylated single chain analog comprises an amino acid selected from the group consisting of a Cys, Lys, Orn, homo-Cys, or Ac-Phe, and the side chain of the amino acid is covalently bonded to a hydrophilic moiety (e.g., PEG). In one embodiment, the acyl group is attached to position B1, B2, B10, or B22 (according to the amino acid numbering of the A and B chains of native insulin), optionally via a spacer comprising Cys, Lys, Orn, homo-Cys, or Ac-Phe.

Alternatively, the acylated first and/or second insulin polypeptide comprises a spacer, wherein the spacer is both acylated and modified to comprise the hydrophilic moiety. Non-limiting examples of suitable spacers include a spacer comprising one or more amino acids selected from the group consisting of Cys, Lys, Om, homo-Cys, and Ac-Phe.

In some embodiments, the amino terminus of at least one N-terminal amino acid of at least one B-chain polypeptides of the insulin receptor partial agonist is modified to comprise a capping group. In another embodiment, the capping group is at amino terminus of the N-terminal amino acid of the B-chain polypeptides of the insulin receptor partial agonist. The capping group may be covalently linked directly to the amino group of the N-terminal amino acid or indirectly to the amino group via a spacer, wherein the spacer is positioned between the amino group of the N-terminal amino acid of the insulin polypeptide and the capping group. The capping group may be an acyl moiety as discussed supra. The capping group substituent may have the general formula RC(O)—, where R is discussed supra. A subembodiment of this aspect of the invention is realized when aspects of RC(O)— may be acetyl, phenylacetyl, 2-(carboxymethoxy)acetyl, carbamoyl, N-alkyl carbamoyl, or alkoxycarbonyl. In an embodiment of this aspect of the invention, the capping group is selected from the group consisting of acetyl, phenylacetyl, carbamoyl, N-alkyl carbamoyl, isobutyl, methoxy acetyl, 2-(carboxymethoxy)acetyl, glycyl, aminoethylglucose (AEG), AEG-C6, PEG1, PEG2, PEG8, N-dimethyl, and alkoxycarbonyl (see Examples herein for structures of the capping group). Carbamolyation of insulin has been disclosed by Oimoni et al., Nephron 46: 63-66 (1987) and insulin dimers comprising a carbamoyl groups at the N-terminus has been disclosed in disclosed in published PCT Application No. WO2014052451 (E.g., MIU-90).

In particular embodiments, at least one N-terminal amino acid is conjugated via the N2 nitrogen to a capping group having the general formula RC(O)—, as defined supra. A subembodiment of this aspect of the invention is realized when aspects of RC(O)— may be acetyl, phenylacetyl, methoxy acetyl, 2-(carboxy methoxy) acetyl, carbamoyl, N-alkyl carbamoyl, glycyl, aminoethylglucose (AEG), AEG-C6, PEG1, PEG2, PEG8, or alkoxycarbonyl, or selected from Capping Group No. 1, 2, 3, 4, 5, 6, 7, and 8.

Exemplary capping groups conjugated to the N-terminal amino group are illustrated in Table II

TABLE II

| Capping | Capping | | | |
|---|---|---|---|---|
| Group | Reagent | Capping Reagent | | Capping |
| No. | Name | Structure | Capping Group Structure | group |
| 1 | 2,5- dioxo- pyrrolidin- 1-yl acetate | 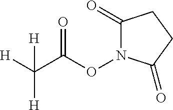 [SMILES: CC(=O)ON1C(=O)CCC1=O] | 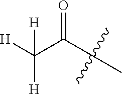 [SMILES: *C(C)=O] | Acetyl |
| 2 | 2,5- dioxo- pyrrolidin- 1-yl 2,5,8,11,14, 17,20,23- octaoxahexa- cosan-26- oate | 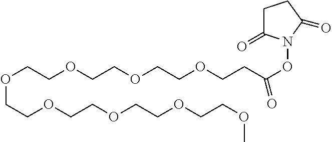 [SMILES: COCCOCCOCCOCCOCCOCCOCCOCCC(=O)ON1C(=O)CCC1=O] | 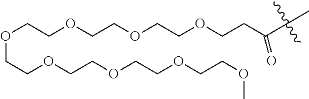 [SMILES: *C(=O)CCOCCOCCOCCOCCOCCOCCOCCOC] | Peg8 |
| 3 | tert-butyl 2- (2-((2,5- dioxo- pyrrolidin- 1-yl)oxy)-2- oxoethoxy) acetate Or diglycolic anhydride | 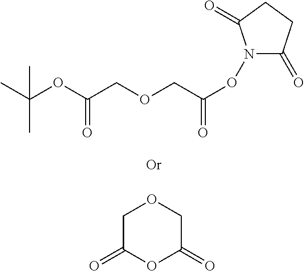 [SMILES: CC(C)(C)OC(=O)COCC(=O)ON1C(=O)CCC1=O ; O=C1COCC(=O)O1] | 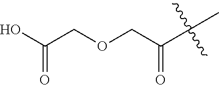 [SMILES: *C(=O)COCC(=O)O] | 2- (carboxy methoxy) acetyl |
| 4 | 2,5- dioxo- pyrrolidin- 1-yl 2- methoxy- acetate | 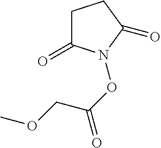 | 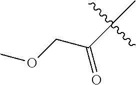 [SMILES: *C(=O)COC] | 2- methoxy acetate or methoxy acetate |
| 5 | Glutaric anhydride | 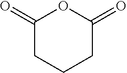 | 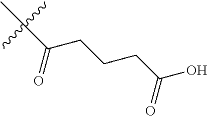 [SMILES: *C(=O)CCCC(=O)O] | glutary1 |
| 6 | Potassium cyanate | KNCO | 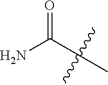 [SMILES: *C(N)=O] | carba- moyl |
| 7 | Formalde- hyde with sodium cyanoboro- hydride | 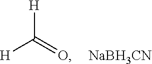 | (CH₃)₂ | Me₂ |
| 8 | 2,5,8,11,14, 17,20,23- octaoxahexa- cosan-26-al, and picoline borane | 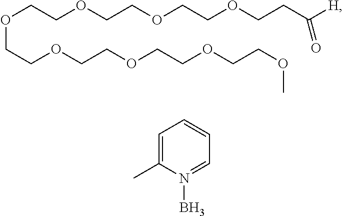 | 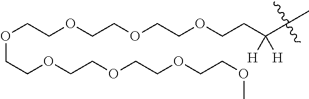 [SMILES: *CCCOCCOCCOCCOCCOCCOCCOCCOC] | 2,5,8,11, 14,17,20, 23- octaoxa- hexacosan- 26-yl |

Thus, an embodiment of this invention is realized when the insulin dimer comprises a capping group conjugated to at least one of the N-terminal amino of each heterodimer B-chain and is selected from the group consisting of acetyl, phenylacetyl, carbamoyl, N-alkyl carbamoyl, isobutyl, methoxy acetyl, 2-(carboxymethoxy) acetyl, glutaric, Me2, carbamoyl, or 2,5,8,11,14,17,20,23-octaoxahexacosan-26-yl, glycine, aminoethylglucose (AEG), AEG-C6, PEG1, PEG2, PEG8, and alkoxycarbonyl. A subembodiment of this aspect of the invention is realized when the capping group is selected from acetyl, phenylacetyl, carbamoyl, N-alkyl carbamoyl, isobutyl, methoxy acetyl, 2-(carboxymethoxy) acetyl, glutaryl, Me2, carbamoyl, and 2,5,8,11,14,17,20,23-octaoxahexacosan-26-yl. Another subembodiment of this aspect of the invention is realized when the capping group is selected from acetyl, phenylacetyl, methoxy acetyl, 2-(carboxymethoxy) acetyl, Me2, and carbamoyl. Another subembodiment of this aspect of the invention is realized when the capping group is selected from Capping Group No. 1, 2, 3, 4, 5, 6, 7, and 8. Another subembodiment of this aspect of the invention is realized when the capping group is acetyl. Another subembodiment of this aspect of the invention is realized when the capping group is phenylacetyl. Another subembodiment of this aspect of the invention is realized when the capping group is methoxy acetyl. Another subembodiment of this aspect of the invention is realized when the capping group is 2-(carboxymethoxy) acetyl. Another subembodiment of this aspect of the invention is realized when the capping group is Me2. Another subembodiment of this aspect of the invention is realized when the capping group is carbamoyl.

Yet another aspect of the invention is realized when capping group is selected from the group consisting of acetyl, phenylacetyl, carbamoyl, N-alkyl carbamoyl, isobutyl, methoxy acetyl, 2-(carboxymethoxy) acetyl, glutaryl, Me2, carbamoyl, and 2,5,8,11,14,17,20,23-octaoxahexacosan-26-yl and the bifunctional linker moiety is selected from the group consisting of Linker 1, Linker 2, Linker 3, Linker 4, Linker 5, Linker 6, Linker 7, Linker 8, Linker 9, Linker 10, Linker 11, Linker 12, Linker 13, Linker 14, Linker 15, and Linker 16. Still another subembodiment of this aspect of the invention is realized when capping group is selected from Capping Group No. 1, 2, 3, 4, 5, 6, 7, and 8. and the bifunctional linker moiety is selected from the group consisting of Linker 1, Linker 2, Linker 3, Linker 4, Linker 5, Linker 6, Linker 7, Linker 8, Linker 9, Linker 10, Linker 11, Linker 12, Linker 13, Linker 14, Linker 15, and Linker 16.

### Exemplary Insulin Dimers

In particular embodiments, the present invention provides insulin dimers wherein a first B29 Lys of a first insulin heterodimer molecule having a first A-chain polypeptide and first B-chain polypeptide and a second B29 Lys of a second insulin heterodimer having a second A-chain polypeptide and second B-chain polypeptide are conjugated together by a bifunctional linker selected from the group consisting Linker 1, Linker 2, Linker 3, Linker 4, Linker 5, Linker 6, Linker 7, Linker 8, Linker 9, Linker 10, Linker 11, Liner 12, Linker 13, Linker 14, Linker 15, and Linker 16.

In particular embodiments, at least one of the B-chain polypeptides is conjugated at its N-terminal amino acid to a capping group as disclosed herein or the N-terminal amino acids of B-chains of both the first insulin heterodimer and second insulin heterodimer are conjugated to a capping group. In particular embodiments, the capping group comprises the general formula RC(O)—, where R is discussed supra. A subembodiment of this aspect of the invention is realized when aspects of RC(O)— may be acetyl, phenylacetyl, methoxy acetyl, 2-(carboxymethoxy)acetyl, carbamoyl, N-alkyl carbamoyl, glycyl, aminoethylglucose (AEG), AEG-C6, PEG1, PEG2, PEG8, or alkoxycarbonyl. Another subembodiment of this aspect of the invention is realized when the capping group is selected from Capping Group No. 1, 2, 3, 4, 5, 6, 7, and 8.

A complete structural depiction of the compounds of this invention is illustrated with insulin dimer 35 in Formula II below, wherein the B29 Lysine of one insulin heterodimer is conjugated to the B29′ Lysine of the other insulin heterodimer through linking moiety, PEG4; disulfide linkages between the Cys₆ and Cys₁₁ residues of the A-chain polypeptide (shown in Formula II) and disulfide linkages between the Cys₇ and Cys₂₀ of the A-chain to the Cys₇ and Cys₁₉ of the B-chain polypeptide, respectively (only shown in Formula II below) exists; the linking moieties are covalently linked to the epsilon amino acid of the lysine residue, wherein the A-chain and A′-chain polypeptide for Dimers 1-43 (Table III) has the amino acid sequence shown in SEQ ID NO: 1; the B-chain and B′-chain polypeptide Dimers 1-43 (Table III) has the amino acid sequence shown in SEQ ID NO:2; and and where the capping group at the terminal nitrogen of each B-chain (i.e., B1 and B1′) for insulin dimer 35 is C(O)CH₃. It should be noted that the linking moiety and capping group in insulin dimer 35 independently may differ from other insulin dimers of the present invention as shown in Table III.

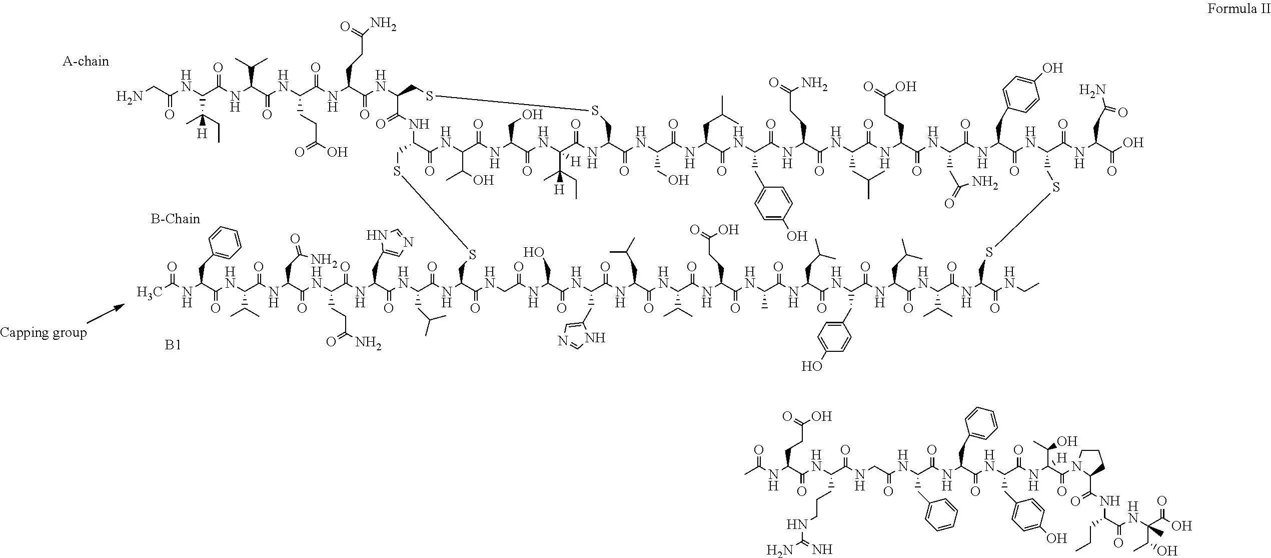

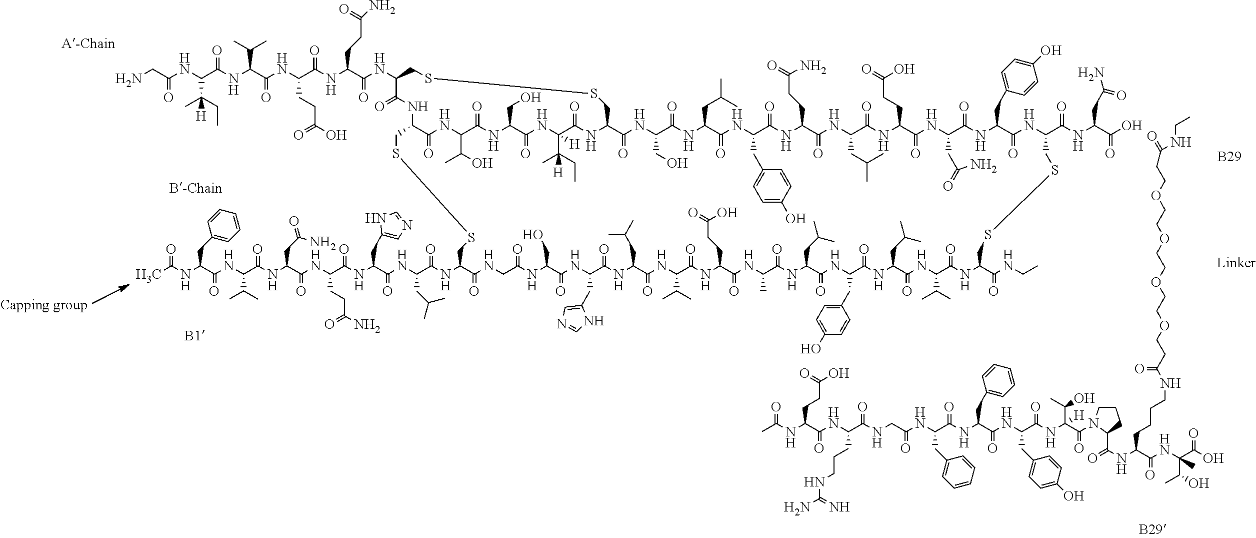

Exemplary insulin dimers include those in Table III:

TABLE III

| Dimer | |
|---|---|
| # | Structure |
| 1 | 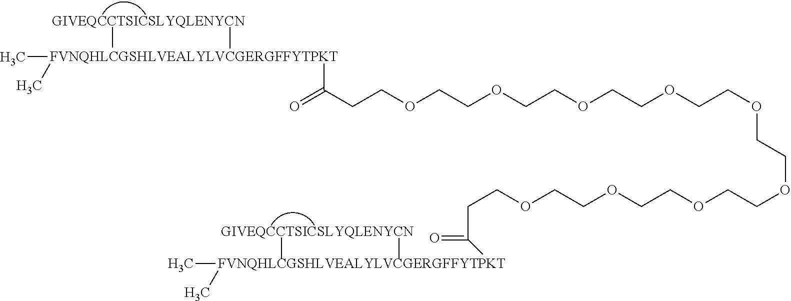 |
| 2 | 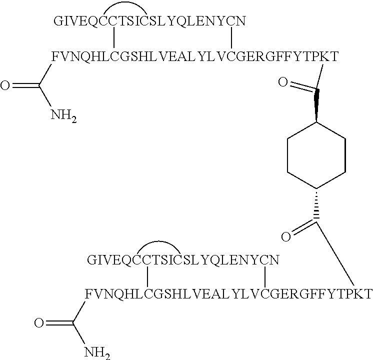 |
| 3 | 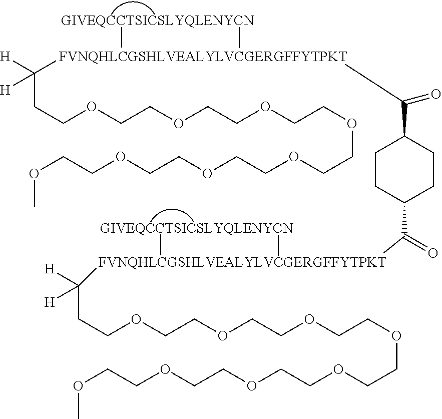 |
| 4 | 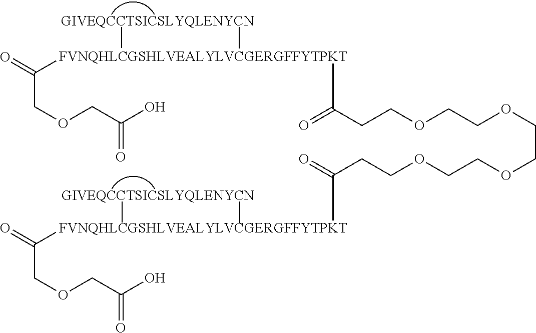 |
| 5 | 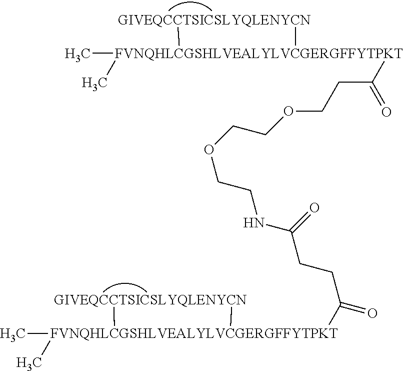 |
| 6 | 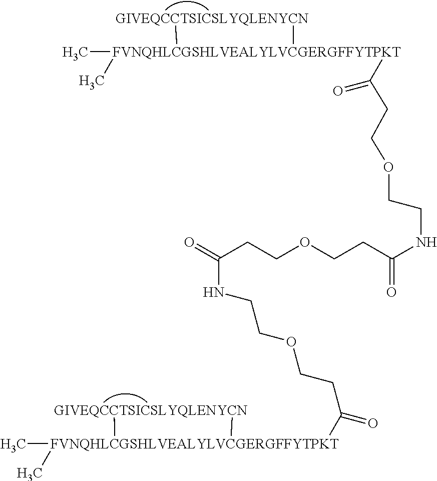 |
| 7 | 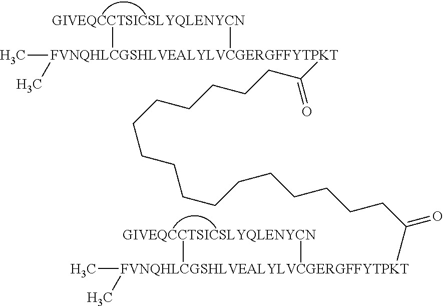 |
| 8 | 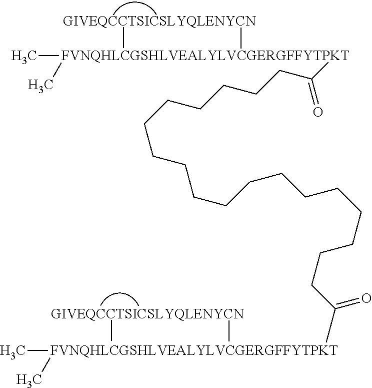 |
| 9 | 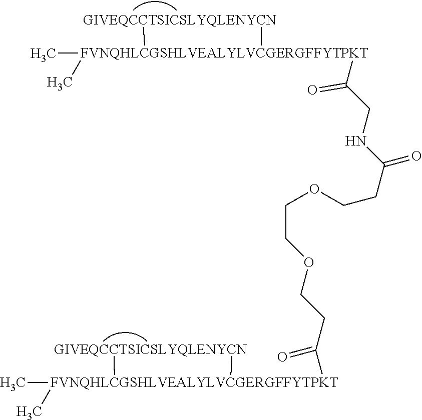 |
| 10 | 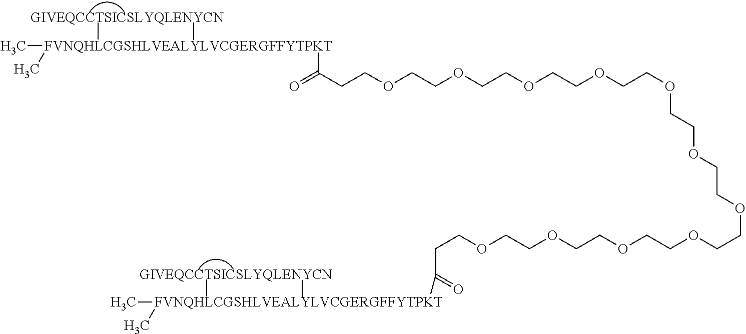 |
| 11 | 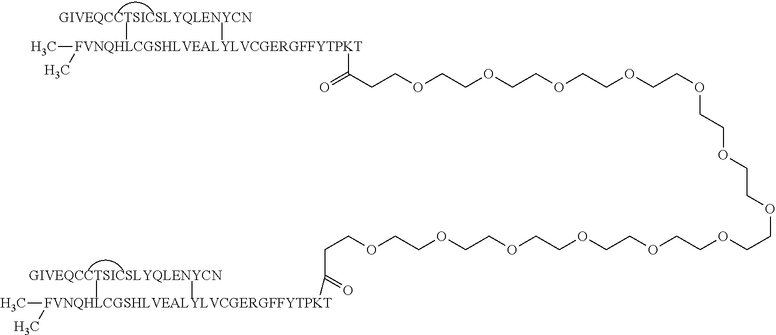 |
| 12 | 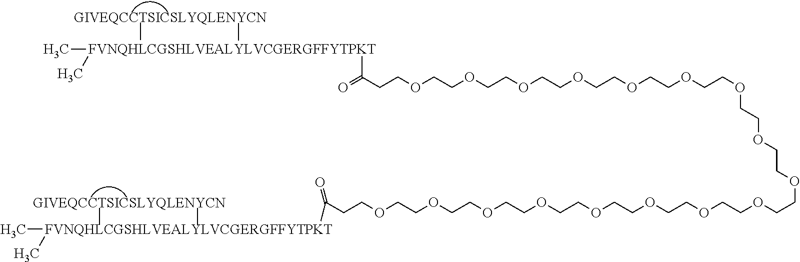 |
| 13 | 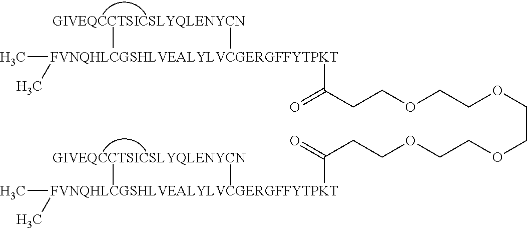 |
| 14 | 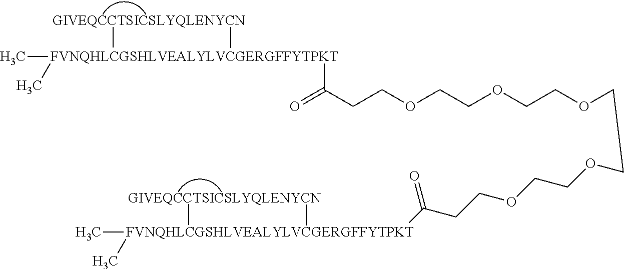 |
| 15 | 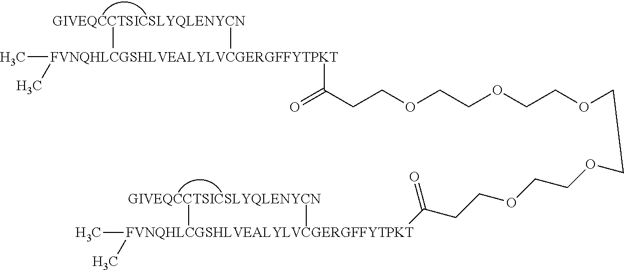 |
| 16 | 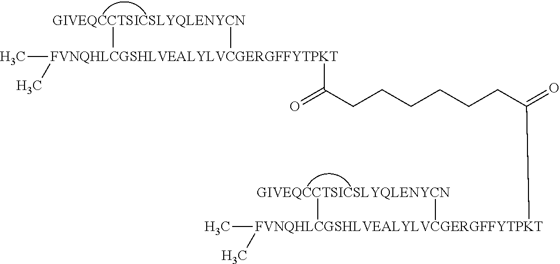 |
| 17 | 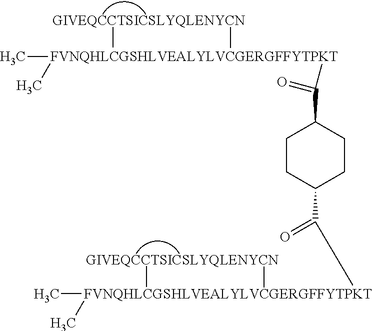 |
| 18 | 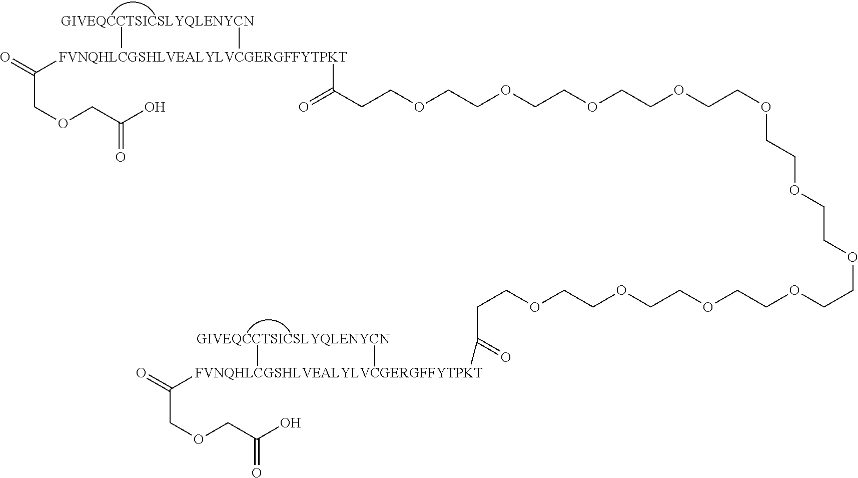 |
| 19 | 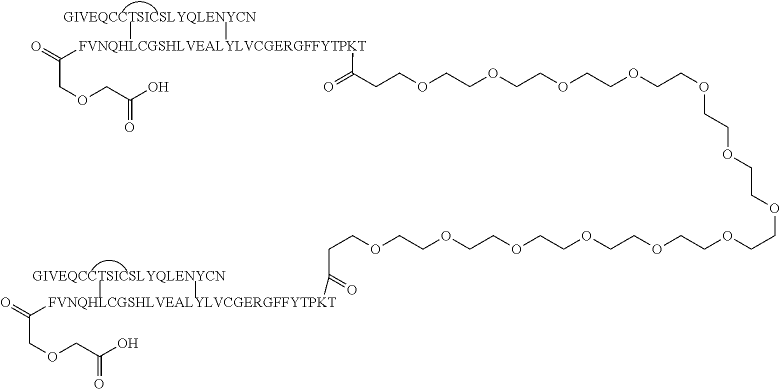 |
| 20 | 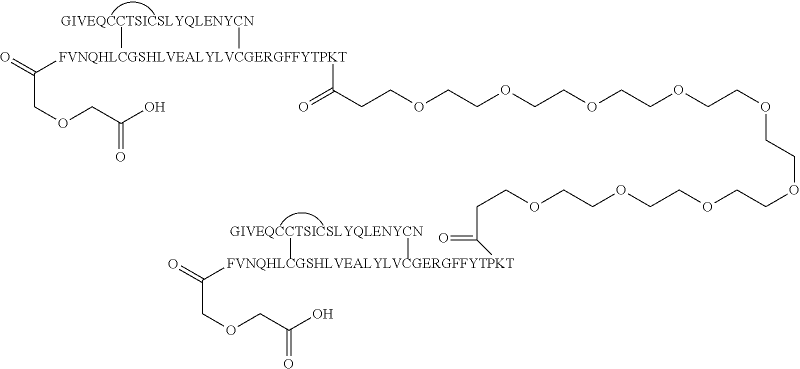 |
| 21 | 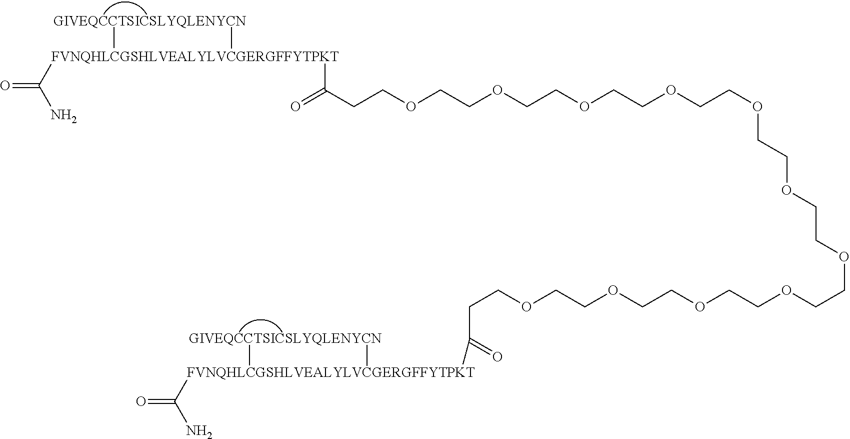 |
| 22 | 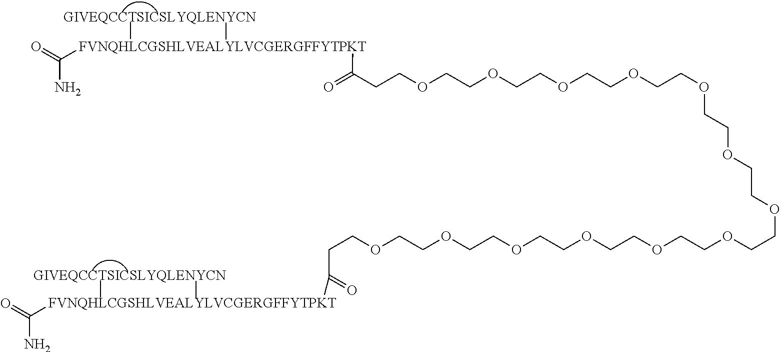 |
| 23 | 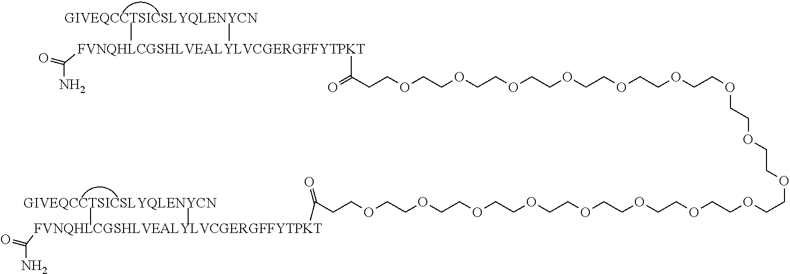 |
| 24 | 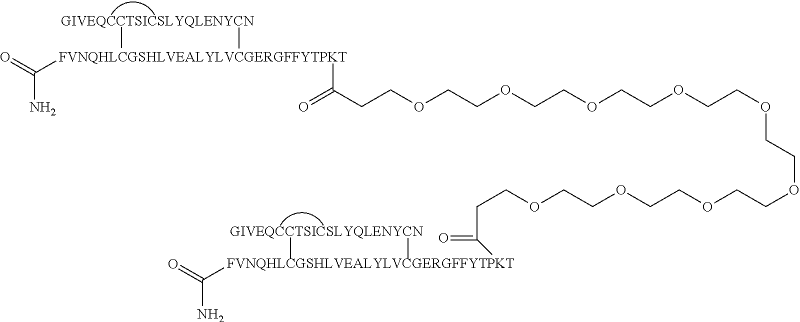 |
| 25 | 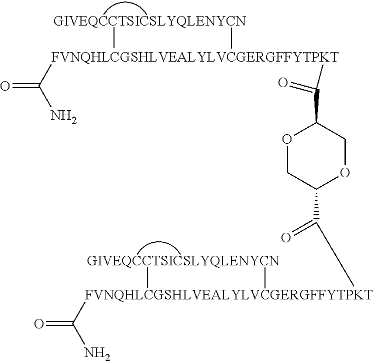 |
| 26 | 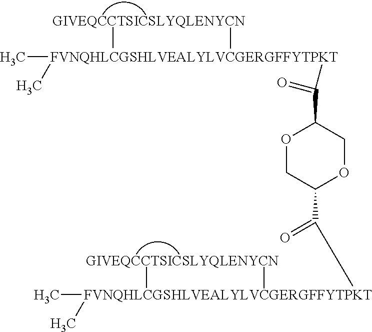 |
| 27 | 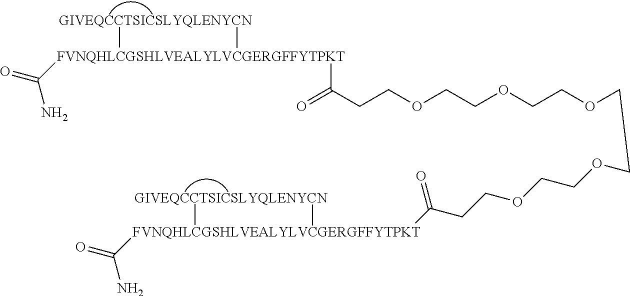 |
| 28 | 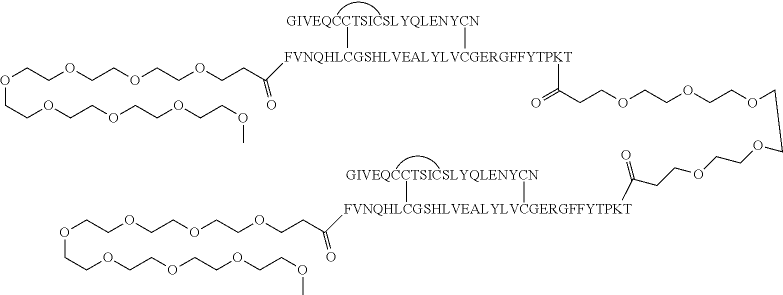 |
| 29 | 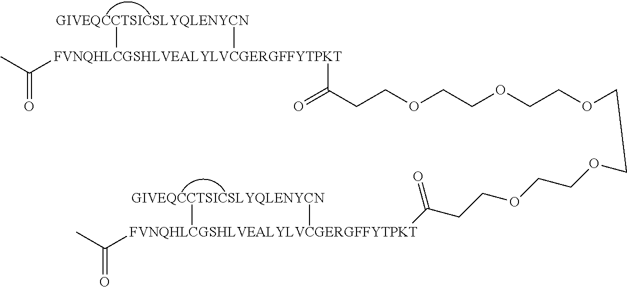 |
| 30 | 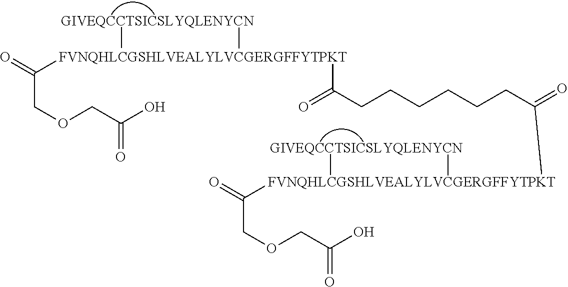 |
| 31 | 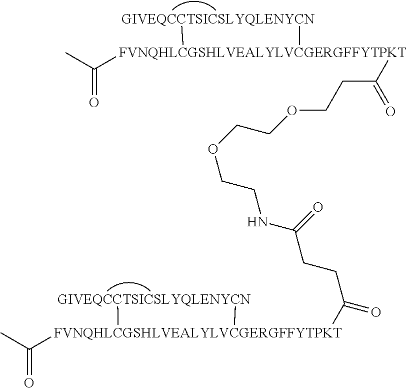 |
| 32 | 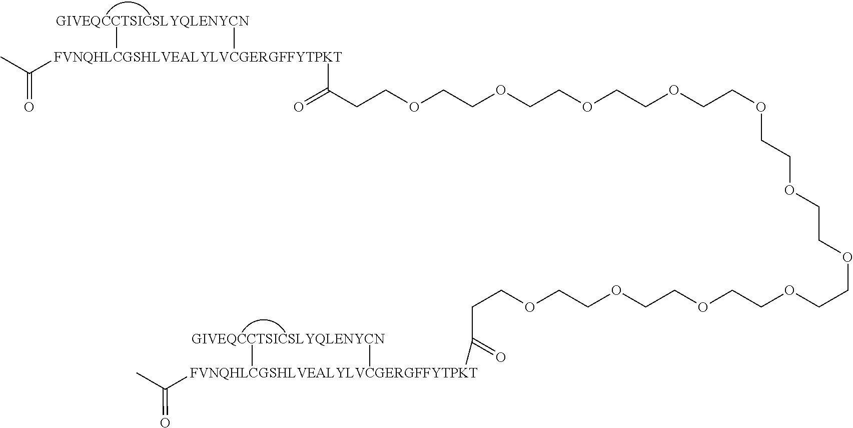 |
| 33 | 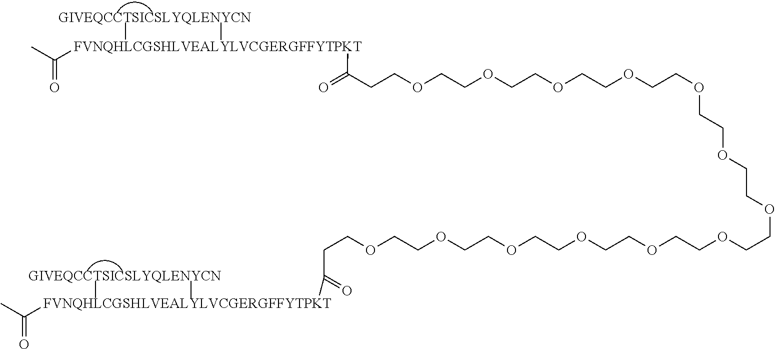 |
| 34 | 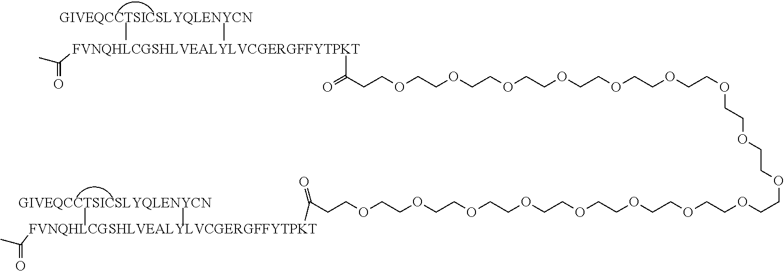 |
| 35 | 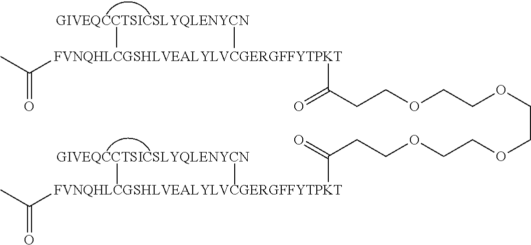 |
| 36 | 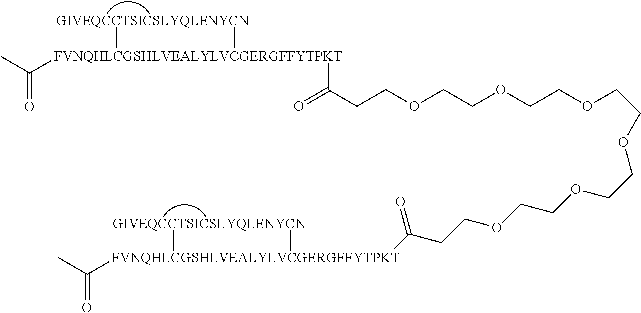 |
| 37 | 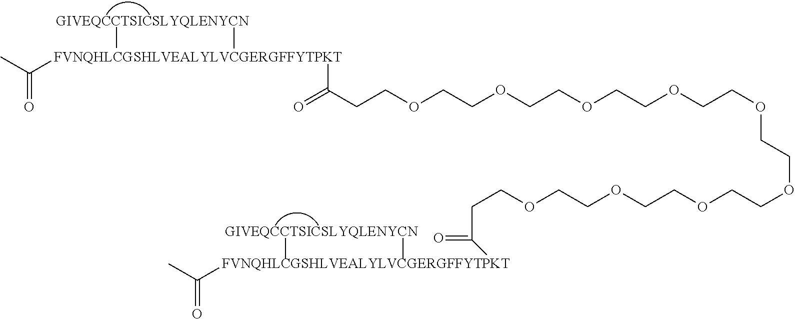 |
| 38 | 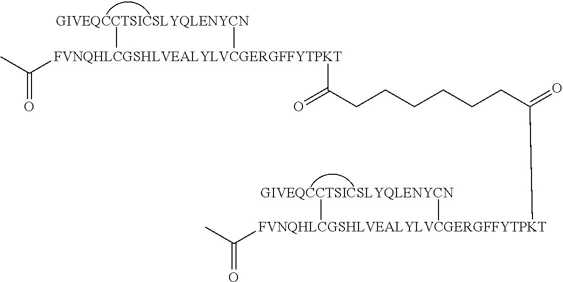 |
| 39 | 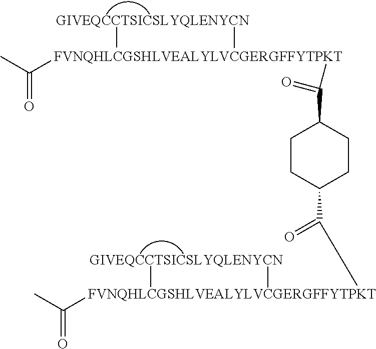 |
| 40 | 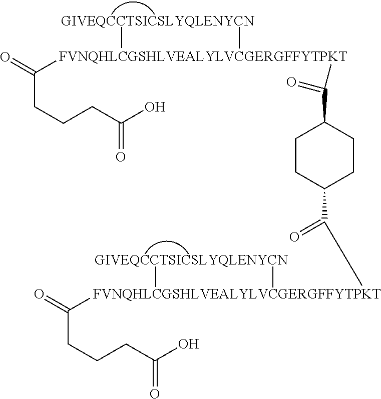 |
| 41 | 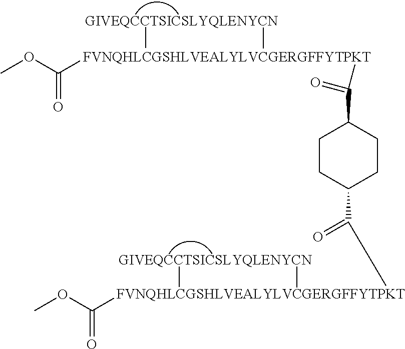 |
| 42 | 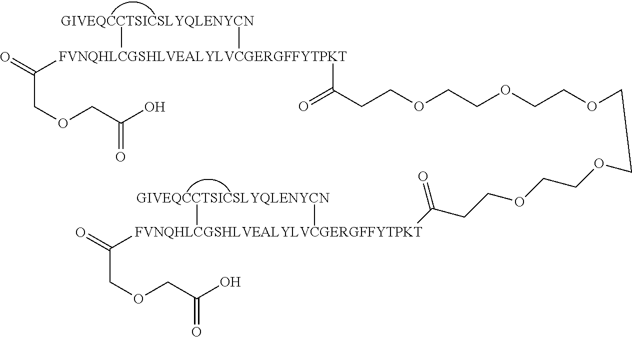 |
| 43 | 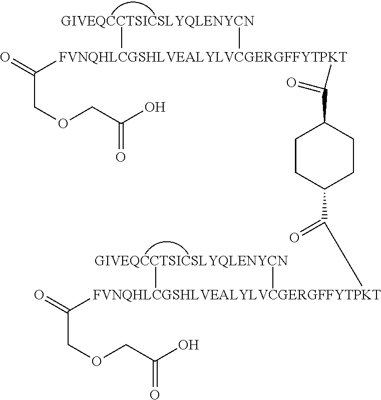 |

### Pharmaceutical Compositions

In accordance with one embodiment a pharmaceutical composition is provided comprising any of the novel insulin dimers disclosed herein, preferably at a purity level of at least 90%, 91%, 92%, 93%, 94%, 95%, 96%, 97%, 98% or 99%, and a pharmaceutically acceptable diluent, carrier or excipient. Such compositions may contain an insulin dimer as disclosed herein at a concentration of at least 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or higher. In one embodiment the pharmaceutical compositions comprise aqueous solutions that are sterilized and optionally stored contained within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

The disclosed insulin dimers are believed to be suitable for any use that has previously been described for insulin peptides. Accordingly, the insulin dimers disclosed herein can be used to treat hyperglycemia, or treat other metabolic diseases that result from high blood glucose levels. Accordingly. the present invention encompasses pharmaceutical compositions comprising a insulin dimers as disclosed herein and a pharmaceutically acceptable carrier for use in treating a patient suffering from high blood glucose levels. In accordance with one embodiment the patient to be treated using a insulin dimer disclosed herein is a domesticated animal. and in another embodiment the patient to be treated is a human.

One method of treating hyperglycemia in accordance with the present disclosure comprises the steps of administering the presently disclosed insulin dimers to a patient using any standard route of administration, including parenterally, such as intravenously, intraperitoneally, subcutaneously or intramuscularly, intrathecally, transdermally, rectally, orally, nasally or by inhalation. In one embodiment the composition is administered subcutaneously or intramuscularly. In one embodiment, the composition is administered parenterally and the insulin polypeptide, or prodrug derivative thereof, is prepackaged in a syringe.

The insulin dimers disclosed herein may be administered alone or in combination with other anti-diabetic agents. Anti-diabetic agents known in the art or under investigation include native insulin, native glucagon and functional analogs thereof, sulfonylureas, such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), or gliclazide (Diamicron); meglitinides, such as repaglinide (Prandin) or nateglinide (Starlix); biguanides such as metformin (Glucophage) or phenformin; thiazolidinediones such as rosiglitazone (Avandia), pioglitazone (Actos), or troglitazone (Rezulin), or other PPARγ inhibitors; alpha glucosidase inhibitors that inhibit carbohydrate digestion, such as miglitol (Glyset), acarbose (Precose/Glucobay); exenatide (Byetta) or pramlintide; Dipeptidyl peptidase-4 (DPP-4) inhibitors such as vildagliptin or sitagliptin; SGLT (sodium-dependent glucose transporter 1) inhibitors; or FBPase (fructose 1,6-bisphosphatase) inhibitors.

Pharmaceutical compositions comprising the insulin dimers disclosed herein can be formulated and administered to patients using standard pharmaceutically acceptable carriers and routes of administration known to those skilled in the art. Accordingly, the present disclosure also encompasses pharmaceutical compositions comprising one or more of the insulin dimers disclosed herein, or a pharmaceutically acceptable salt thereof, in combination with a pharmaceutically acceptable carrier. For example, the pharmaceutical compositions comprising the insulin dimers disclosed herein may optionally contain zinc ions, preservatives (e.g., phenol, cresol, parabens), isotonicizing agents (e.g., mannitol, sorbitol, lactose, dextrose, trehalose, sodium chloride, glycerol), buffer substances, salts, acids and alkalis and also further excipients. These substances can in each case be present individually or alternatively as mixtures. Glycerol, dextrose, lactose, sorbitol and mannitol are customarily present in the pharmaceutical preparation in a concentration of 100-250 mM, NaCl in a concentration of up to 150 mM. Buffer substances, such as, for example, phosphate, acetate, citrate, arginine, glycylglycine or TRIS (i.e. 2-amino-2-hydroxymethyl-1,3-propanediol) buffer and corresponding salts, are present in a concentration of 5-250 mM, commonly from about 10-100 mM. Further excipients can be, inter alia, salts or arginine.

In one embodiment the pharmaceutical composition comprises a 1 mg/mL concentration of the insulin dimer at a pH of about 4.0 to about 7.0 in a phosphate buffer system. The pharmaceutical compositions may comprise the insulin dimer as the sole pharmaceutically active component, or the insulin dimer can be combined with one or more additional active agents. All therapeutic methods, pharmaceutical compositions, kits and other similar embodiments described herein contemplate that insulin dimers include all pharmaceutically acceptable salts thereof.

In one embodiment the kit is provided with a device for administering the insulin dimers composition to a patient. The kit may further include a variety of containers, e.g., vials, tubes, bottles, and the like. Preferably, the kits will also include instructions for use. In accordance with one embodiment the device of the kit is an aerosol dispensing device, wherein the composition is prepackaged within the aerosol device. In another embodiment the kit comprises a syringe and a needle, and in one embodiment the insulin dimer composition is prepackaged within the syringe.

The compounds of this invention may be prepared by standard synthetic methods, recombinant DNA techniques, or any other methods of preparing peptides and fusion proteins. Although certain non-natural amino acids cannot be expressed by standard recombinant DNA techniques, techniques for their preparation are known in the art. Compounds of this invention that encompass non-peptide portions may be synthesized by standard organic chemistry reactions, in addition to standard peptide chemistry reactions when applicable.

The following examples are intended to promote a further understanding of the present invention.

### EXAMPLES

### General Procedures

All chemicals were purchased from commercial sources, unless otherwise noted. Reactions were usually carried out at ambient temperature or at room temperature unless otherwise noted. Reactions sensitive to moisture or air were performed under nitrogen or argon using anhydrous solvents and reagents. The progress of reactions was monitored by analytical thin layer chromatography (TLC) reagents, and ultra performance liquid chromatography-mass spectrometry (UPLC-MS). TLC was performed on E. Merck TLC plates precoated with silica gel 60F-254, layer thickness 0.25 mm. The plates were visualized using 254 nm UV and/or by exposure to cerium ammonium molybdate (CAM) or p-anisaldehyde staining solutions followed by charring. Ultra performance liquid chromatography (UPLC) was performed on a Waters Acquity™ UPLC® system.
Method A: Waters Acquity™ UPLC® BEH C18 1.7 μm 1.0×50 mm column with gradient 10:90-95:5 v/v CH₃CN/H₂O+v 0.05% TFA over 2.0 min; flow rate 0.3 mL/min, UV wavelength 215 nm; UPLC-MS;
Method B: Waters Acquity™ UPLC® BEH C18 1.7 μm 2.1×100 mm column with gradient 20:80-90:10 v/v CH₃CN/H₂O+v 0.05% TFA over 4.0 min and 90:10-95:5 v/v CH₃CN/H₂O+v 0.05% TFA over 0.5 min; flow rate 0.3 mL/min, UV wavelength 200-300 nm; UPLC-MS;
Method C: Waters Acquity™ UPLC® BEH C8 1.7 μm 2.1×100 mm column with gradient 10:90-55:45 v/v CH₃CN/H₂O+v 0.05% TFA over 4.0 min and 55:45-95:5 v/v CH₃CN/H₂O+v 0.05% TFA over 40 sec; flow rate 0.3 mL/min, UV wavelength 200-300 nm; UPLC-MS;
Method D: Waters Acquity™ UPLC® BEH C18 1.7 μm 1.0×50 mm column with gradient 10:90-95:5 v/v CH₃CN/H₂O+v 0.05% TFA over 5.0 min; flow rate 0.3 mL/min, UV wavelength 215 nm; UPLC-MS;
Method E: Waters Acquity™ UPLC® BEH C8 1.7 μm 2.1×100 mm column with gradient 10:90-55:45 v/v CH₃CN/H₂O+v 0.05% TFA over 14.0 min and 55:45-95:5 v/v CH₃CN/H₂O+v 0.05% TFA over 40 sec; flow rate 0.3 mL/min, UV wavelength 200-300 nm; UPLC-MS;
Method F: Waters Acquity™ UPLC® BEH C8 1.7 μm 2.1×100 mm column with gradient 20:80-90:10 v/v CH₃CN/H₂O+v 0.1% TFA over 4.0 min and 90:10-95:5 v/v CH₃CN/H₂O+v 0.1% TFA over 0.4 min; flow rate 0.3 mL/min, UV wavelength 200-300 nm.

Mass analysis was performed on a Waters SQ Detector with electrospray ionization in positive ion detection mode and the scan range of the mass-to-charge ratio was 170-900 or a Waters Micromass® LCT Premier™ XE with electrospray ionization in positive ion detection mode and the scan range of the mass-to-charge ratio was 300-2000. The identification of the produced insulin conjugates or IRPA was confirmed by comparing the theoretical molecular weight to the experimental value that was measured using UPLC-MS. For the determination of the linkage positions. specifically. insulin dimers were subjected to DTT treatment (for a/b chain) or Glu-C digestion (with or without reduction and alkylation), and then the resulting peptides were analyzed by LC-MS. Based on the measured masses. the linkage positions were deduced.

Flash chromatography was performed using either a Biotage Flash Chromatography apparatus (Dyax Corp.) or a CombiFlash® Rf instrument (Teledyne Isco). Normal-phase chromatography was carried out on silica gel (20-70 μm, 60 Å pore size) in pre-packed cartridges of the size noted. Ion exchange chromatography was carried out on a silica-based material with a bonded coating of a hydrophilic, anionic poly (2-sulfoethyl aspartamide) (PolySULFOETHYL A column, PolyLC Inc., 250×21 mm, 5 m, 1000 Å pore size). Reverse-phase chromatography was carried out on C18-bonded silica gel (20-60 μm, 60-100 Å pore size) in pre-packed cartridges of the size noted. Preparative scale HPLC was performed on Gilson 333-334 binary system using Waters DELTA PAK C4 15 μm, 300 Å, 50×250 mm column or KROMASIL® C8 10 μm, 100 Å, 50×250 mm column, flow rate 85 mL/min, with gradient noted. Concentration of solutions was carried out on a rotary evaporator under reduced pressure or freeze-dried on a VirTis Freezemobile Freeze Dryer (SP Scientific).

Abbreviations: acetonitrile (AcCN), aqueous (aq), 1-[bis(dimethylamino)methylene]-1H-1,2,3-triazolo[4,5-b]pyridinium 3-oxid hexafluorophosphate (HATU), dichloromethane (DCM), 4-dimethylaminopyridine (DMAP), N,N-diisopropylethylamine or Hünig's base (DIPEA), N,N-dimethylacetamide (DMA), N,N-dimethylformamide (DMF), dimethyl sulfoxide (DMSO), ethyl acetate (EtOAc), N-(3-dimethylaminopropyl)-N′-ethylcarbodiimide hydrochloride (EDC), gram(s) (g), 1-hydroxy benzotriazole hydrate (HOBt), hour(s) (h or hr), isopropyl acetate (IPAc), mass spectrum (ms or MS), methyl tert-butyl ether (MTBE), microgram(s) (g), microliter(s) (μL), micromole (mol), milligram(s) (mg), milliliter(s) (mL), millimole (mmol), minute(s) (min), retention time (t_{R}), room temperature (rt, or Rt), saturated (sat. or sat'd), saturated aq sodium chloride solution (brine), 1,1,3,3-tetramethylguanidine (TMG), 2,2,6,6-tetramethylpiperidine (TMP), triethylamine (TEA), trifluoroacetic acid (TFA), N,N,N′,N′-tetramethyl-O-(N-succinimidyl)uronium tetrafluoroborate (TSTU), ?Pearlman's catalyst: 20% palladium hydroxide on carbon, N-(terf-Butoxycarbonloxy)succinimide (BOC-OSU), Triisopropylsilane (iPr₃SiH), Phenylacetyl (PhAc), diethyl ether (Et2O).

The term “RHI” refers to recombinant human insulin and is used to indicate that the insulin has the amino acid sequence characteristic of native, wild-type human insulin. As used herein in the tables, the term indicates that the amino acid sequence of the insulin comprising the dimer is that of native, wild-type human insulin.

Linking Reagents 1 through 9 and capping groups such as 1-2 and 4-8 are commercially available and can be purchased, for example, from Sigma-Aldrich and/or Quanta Biodesign LTD (Plain City, Ohio, www.quantabiodesign.com).

### Preparative Example 1—Tert-butyl 2-(2-((2,5-dioxopyrrolidin-1-yl)oxy)-2-oxoethoxy)acetate (Capping Reagent 3)

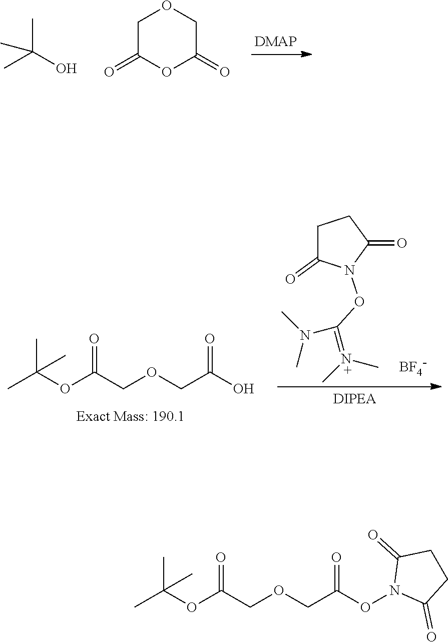

### Step 1. 2-(2-(Tert-butoxy)-2-oxoethoxy)acetic acid

To a solution of diglycolic anhydride (1.1 g, 9.48 mmol) in tert-butanol (5 mL), was added DMAP (0.116 g, 0.948 mmol), and the resulting mixture was stirred at 40° C. for 24 h. The reaction mixture was cooled down to room temperature and concentrated under reduced pressure to dryness, and re-dissolved in 100 mL of 0.1 N HCl. The product was then extracted into DCM (3×40 mL). The combined organic phase was washed with water, brine, dried over Na₂SO₄, filtered and concentrated. The residue was purified by column chromatography on 24 g ISCO silica gel column eluting with a gradient of hexanes-ethyl acetate to furnish 2-(2-(tert-butoxy)-2-oxoethoxy) acetic acid. ¹H NMR (500 MHz, CDCl₃): δ 4.23 (s; 2H); 4.14 (s; 2H); 1.50 (s; 9H).

### Step 2. Tert-butyl 2-(2-((2,5-dioxopyrrolidin-1-yl)oxy)-2-oxoethoxy)acetate

To a solution of 2-(2-(tert-butoxy)-2-oxoethoxy)acetic acid (760 mg, 4.00 mmol) in DCM (25 ml) at 0° C. was added TSTU (1.3 g, 4.32 mmol) and DIPEA (0.85 ml, 4.87 mmol). The reaction mixture was stirred at room temp for 2 h and quenched with TFA (0.45 mL, 5.84 mmol). The residue was purified by column chromatography on 40 g silica gel Column, eluting with a gradient of hexanes-ethyl acetate to furnish the title material as a solid. ¹H NMR (500 MHz, CDCl₃): δ 4.60 (s; 2H); 4.14 (s; 2H); 2.86 (s; 4H); 1.49 (s; 9H).

### Preparative Example 2

### Bis(2,5-dioxopyrrolidin-1-yl) octadecanedioate (Linking Reagent 10)

To a solution of octadecanedioic acid (1.0 g, 3.18 mmol) and TSTU (2.0 g, 6.64 mmol) in DCM (12.0 mL) was added DIPEA (1.1 mL, 6.30 mmol) and the resulting mixture was stirred at rt for 1 h. The precipitated product was filtered and washed with DCM (3×5 mL) to obtain the title material as a white solid. UPLC-MS Method A, Rt=1.56 min, m/z=509.4 [M+1].

### Preparative Example 3

### Bis(2,5-dioxopyrrolidin-1-yl) icosanedioate (Linking Reagent 11)

To a solution of icosanedioic acid (1.0 g, 2.92 mmol) and TSTU (1.80 g, 5.98 mmol) in DCM (12.0 mL) was added DIPEA (1.0 mL, 5.73 mmol) and the resulting mixture was stirred at rt for 1 h. The precipitated product was filtered and washed with DCM (3×5 mL) to obtain the title material as a solid. UPLC-MS Method A, Rt=1.70 min, m/z=537.5 [M+1].

### Preparative Example 4

### Bis(2,5-dioxopyrrolidin-1-yl) (2S,5R)-1,4-dioxane-2,5-dicarboxylate (Linking Reagent 12)

To a solution of trans-1,4-dioxane-2,5-dicarboxylic acid (Summerbell et al., JACS 1954, 76, 6401) (41.4 mg, 0.235 mmol) and TSTU (159 mg, 0.528 mmol) in DMF (1.0 mL) at 0° C. was added DIPEA (86 μl, 0.492 mmol) and the resulting mixture was stirred overnight. The crude mixture was quenched with TFA (45 μl, 0.584 mmol) and directly purified on ISCO C18 column (water-AcN). The material was re-purified on 24g ISCO SiO₂ column, eluting with 100% hexanes (3 column volumes); then 0-100% EtOAc in Hexanes (8 column volumes) and 100% EtOAc (5 column volumes) to give the title product as a solid. NMR dmso-d6 2.08 (m, 2H), 4.25 (m, 2H), 3.98 (m, 2H), 2.85 (s, 8H).

### Preparative Example 5

### 2,5-dioxopyrrolidin-1-yl 3-(2-(3-((2-((2,5-dioxopyrrolidin-1-yl) oxy)-2-oxoethyl)amino)-3-oxopropoxy)ethoxy)propanoate (Linking Reagent 16)

### Step 1: Benzyl 3-(2-(3-((2-(benzyloxy)-2-oxoethyl) amino)-3-oxopropoxy) ethoxy)propanoate

To a solution of 3,3′-(ethane-1,2-diylbis(oxy))dipropionic acid (536 mg, 1.809 mmol) and TSTU (572 mg, 1.899 mmol) in DMF (3.0 mL) was added DIPEA (0.5 mL). The resulting mixture was stirred at rt for 60 minutes, followed by the addition of 2-(benzyloxy)-2-oxoethanaminium chloride (365 mg, 1.809 mmol) as a solid and DIPEA (0.65 mL) and stirring continued at room tempearture over weekend. The reaction mixture was concentrated and residue was purified by column chromatography on 40 g SiO2 ISCO column, eluting with 100% Hexanes (2 column volumes); then 0-100% EtOAc in Hexanes (6 column volumes), 100% EtOAc (6 column volumes) to give the title material as an oil. UPLC-MS Method B, Rt=3.13 min, m/z=444.0 [M+1].

### Step 2: 3-(2-(3-((carboxymethyl)amino)-3-oxopropoxy)ethoxy)propanoic acid

A mixture of Benzyl 3-(2-(3-((2-(benzyloxy)-2-oxoethyl)amino)-3-oxopropoxy)ethoxy)propanoate (600 mg, 1.353 mmol) and Pearlman's Catalyst (63.5 mg, 0.090 mmol) was suspended in acetone (5.0 mL) and water (3.0 mL and allowed to stir under a balloon of hydrogen for 2 h. The catalyst was filtered off and washed with acetone (3×4.0 mL). The filtrate was concentrated to obtain the title material as a gel. UPLC-MS Method A, Rt =0.32 min, m/z=264.15 [M+1].

### Step 3: 2,5-dioxopyrrolidin-1-yl 3-(2-(3-((2-((2,5-dioxopyrrolidin-1-yl)oxy)-2-oxoethyl)amino)-3-oxopropoxy)ethoxy)propanoate

To a solution of 3-(2-(3-((carboxymethyl)amino)-3-oxopropoxy)ethoxy)propanoic acid (330 mg, 1.25 mmol) and TSTU (793 mg, 2.63 mmol) in DMF (2 mL) was added DIPEA (0.46 mL, 2.64 mmol) and the resulting mixture was stirred at rt for 1 h. Crude mixture was quenched with TFA (0.3 mL, 3.92 mmol) and directly purified on 40 g c18 ISCO Column, eluting with 10% ACN in H2O (3 column volumes); then 10-100% ACN in H₂O (8 column volumes), 100% ACN (5 column volumes) to give the title material as a solid. UPLC-MS Method A, Rt=0.51 min, m/z=458.2 [M+1].

### Preparative Example 6

### bis(2,5-dioxopyrrolidin-1-yl) trans-cyclohexane-1,4-dicarboxylate (trans-cyclohexane 1,4-diacid, Linking Reagent 13)

To a solution of trans-cyclohexane-1,4-dicarboxylic acid (200 mg, 1.162 mmol) in DCM (11 mL) at 0° C. was added TSTU (734 mg, 2.439 mmol) and DIPEA (0.5 mL, 2.86 mmol). The resulting reaction mixture was stirred at rt for 1 hr. The product was crushed out in reaction solution as a white solid; filtered and washed with DCM (2×5 ml); and dried in vacuo to obtain the title compound. UPLC-MS calculated for C₁₆H₁₈N₂O₈, 366.11, observed m/z: 367.16 [M+1], t_{R}=3.20 min, using UPLC-MS Method A. ¹H NMR (500 MHz, DMSO): δ 2.81-2.89 (m; 2H); 2.80 (s; 8H); 2.02-2.10 (m; 4H); 1.57-1.63 (m; 4H).

### Preparative Example 7

### 2,5-dioxopyrrolidin-1-yl 4-((2-(2-(3-((2,5-dioxopyrrolidin-1-yl)oxy)-3-oxopropoxy)ethoxy)ethyl)amino)-4-oxobutanoate (Linking Reagent 14)

### Step 1. 4-((2-(2-(2-carboxyethoxy)ethoxy)ethyl)amino)-4-oxobutanoic acid

To a solution of tert-butyl 3-(2-(2-aminoethoxy)ethoxy)propanoate (250 mg, 1.072 mmol) in DMF (1.5 mL) at rt was added dihydrofuran-2,5-dione (107 mg, 1.072 mmol), and followed by TEA (0.149 mL, 1.072 mmol). The mixture was stirred at rt for 3 hr and then concentrated to remove DMF. To the resulting residue at 0° C. was added 1 mL TFA, and the mixture was allowed to stir at rt over night. After overnight, the solution was concentrated to give the diacid, which was used in the next step without further purificaiton. UPLC-MS Method A: t_{R}=0.4 min, 278 [M+1].

### Step 2. 2,5-dioxopyrrolidin-1-yl 4-((2-(2-(3-((2,5-dioxopyrrolidin-1-yl)oxy)-3-oxopropoxy)ethoxy)ethyl)amino)-4-oxobutanoate

To a solution of the product of Step 1 (297 mg, 1.071 mmol) in DMF (1.5 mL) at rt was added TSTU (661 mg, 2.196 mmol), and followed by DIPEA (0.384 mL, 2.196 mmol). The mixture was stirred at rt for 3 hr and concentrated. The residue was purified by reverse phase chromatography using 120 g C18 column, elute with 0-40% AcCN in H₂O. Fractions containing the product were combined and freeze-dried to give title compound. UPLC-MS Method C: t_{R}=3.73 min, 472 [M+1].

### Preparative Example 8

### Bis(2,5-dioxopyrrolidin-1-yl)8,14-dioxo-4,11,18-trioxa-7,15-diazahenicosanedioate (Linking Reagent 15)

### Step 1. 8,14-dioxo-4,11,18-trioxa-7,15-diazahenicosane-1,21-dioic acid

To the solution of tert-butyl 3-(2-aminoethoxy)propanoate (246 mg, 1.302 mmol) in DMF (1.5 mL) at rt was added bis (2,5-dioxopyrrolidin-1-yl) 3,3′-oxydipropanoate (232 mg, 0.651 mmol), followed by Hunig's Base (0.284 mL, 1.628 mmol). The mixture was stirred at RT overnight, concentrated down to dryness, treated at 0° C. with 1.0 mL of TFA, then stirred overnight. UPLC indicated the desired product Rt=0.71 min, m/e=393.3 [M+H]⁺

### Step 2. Bis(2,5-dioxopyrrolidin-1-yl) 8,14-dioxo-4,11,18-trioxa-7,15-diazahenicosanedioate

To the solution of 8,14-dioxo-4,11,18-trioxa-7,15-diazahenicosane-1,21-dioic acid (256 mg, 0.652 mmol) in DMF (1.5 mL) at rt was added TSTU (403 mg, 1.337 mmol), followed by Hunig's Base (0.234 mL, 1.337 mmol). The mixture was stirred ar rt overnight, then concentrated.and purified by Biotage snap on 120 g C18 column, eluting with 0-30% ACN in water, 20 CV. Combined fractions and lyophilized to powder. Method C: Rt=2.52 min, m/e=586.8 [M+H]⁺

### Synthesis of Monomeric Insulin Analogs

### Preparative Example 9: Analog 1. A1=PhAc-RHI monomer

synthesis of phenylacetate modified insulin is described. The modified insulin comprises the insulin A chain polypeptide (SEQ ID NO:1) conjugated to phenylacetate at the N-terminal amino group and the insulin B chain polypeptide (SEQ ID NO:2) conjugated to phenylacetate a the N-terminal amino group and the epsilon amino group of lysine at position 29.

### Preparation of A1-(phenylacetyl)insulin 3d using PGA-080-C-His

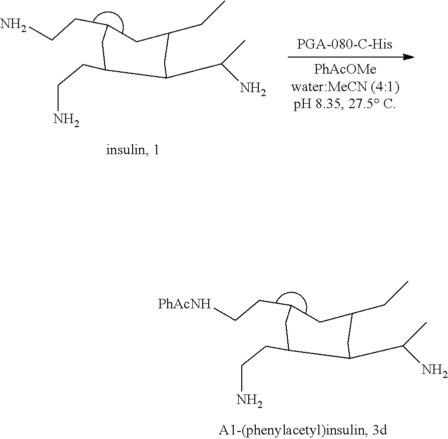

To a 5 L vessel with an overhead stirrer recombinant human insulin (purchased from Sigma-Aldrich)) (represented as 1, 213.7 g. 94 wt %, 34.4 mmol) and water (3 L) were charged and the mixture was warmed up to at 27.5° C. The pH of the suspension was adjusted to pH 8.5 with 2M NaOH (50 mL, 566 mmol, 16.5 eq.). Acetonitrile (850 mL) was added followed by methyl phenylacetate (98 mL, 688 mmol, 20 eq. The reaction was commenced by the addition of the PGA-080-C-His (described and prepared as SEQ ID No. 140 in patent publication USSN2018/0187180, incorporated herein by reference in its entirety) solution in water (4.53 g in 450 mL, 0.45 μm filtered) and the pH was maintained at 8.35 with 2M NaOH using a Metrohm pH-stat system. Upon the reaction completion after about 6 hours, the mixture was transferred to a 10 L cylindrical vessel and diluted with water (8.5 L). An aqueous solution of sodium acetate (450 mL, 1M, pH 5) was added at a rate of 300 mL/h at 27.5° C. to reach pH 6.0. The resulting white slurry was aged for an additional 1 hour, filtered and washed with an aqueous solution of sodium acetate (1.5 L, 0.5 M, pH 6). The crude product was suction dried in the air in the filter for 1 h then slurry washed with an IPAC/t-amyl alcohol solution (2:1, 3×600 mL). The solid was then vacuum dried with nitrogen sweep to yield A1-(phenylacetyl)insulin (3d).

### Preparative Example 10—Analog 2. A1=PhAc, B1=Me₂N-RHI Monomer

To suspended Analog 1 (1000 mg, 0.169 mmol) in water (20.0 mL) and adjusted pH to 4.0 using AcOH (added in increments of 100 μL) was added 1.0 mL of AcN to improve solubility. Formaldehyde (37% wt, aqueous solution) (33.8 mg, 0.338 mmol) and sodium cyanoborohydride (21.21 mg, 0.338 mmol)) as a solid were added. The milky reaction mixture was stirred over 4 hrs. AcN (˜30% by volume) was added and adjusted pH to 2.5. The product was isolated by reverse-phase chromatography on C-8 phase (Column Kromasil, C8 10 uM 100 A, size 250×50 mm; solvent A=water/0.05% TFA, solvent B=AcN/0.05% TFA), Flow=85 mL/min, gradient B in A 27-38% in 30 min, UPLC-MS Method C, Rt=3.72 min, m/z=1489.5 [(M+4)/4].

### Preparative Example 11—Analog 3. A1=PhAc, B1=Carbamoyl-RHI Monomer

A mixture of Analog 1 (500 mg, 0.084 mmol) in 25.5 mL H₂O containing potassium phosphate dibasic (149.4 mg, 0.858 mmol) was adjusted to pH 7.4. A solution of potassium cyanate (80 mg, 0.986 mmol) in 2.5 mL of H₂O was added and the reaction mixture was stirred overnight. The product was isolated by reverse-phase chromatography on C-8 phase (Column Kromasil, C8 10 uM 100 A, size 250×50 mm; solvent A=water/0.05% TFA, solvent B=AcN/0.05% TFA), Flow=85 mL/min, gradient B in A 29-35% in 30 min, UPLC-MS Method C, Rt=3.77 min, m/z=1493.42 [(M+4)/4].

### Preparative Example 12—Analog 4. A1=PhAc, B1=2,5,8,11,14,17,20,23-octaoxahexacosan-26-yl RHI

To a solution of Analog 1 (100 mg, 0.017 mmol) in water (2.0 mL) was added acetic acid (0.097 mL, 1.688 mmol). The resulting pH was assured to be 4.3. The aldehyde, 2,5,8,11,14,17,20,23-octaoxahexacosan-26-al (13.38 mg, 0.034 mmol) was added followed by 2-picoline borane complex (7.22 mg, 0.068 mmol) as a solid. The mixture was stirred for 4 hrs. The product was purified by reverse-phase chromatography on C-8 phase (Column Kromasil, C8 10 uM 100 A, size 250×50 mm; solvent A=water/0.05% TFA, solvent B=AcN/0.05% TFA), Flow=85 mL/min, gradient B in A 26-35% in 30 min. UPLC-MS Method C, Rt=3.73 min, m/z=1578.31 [(M+4)/4].

### Preparative Example 13 Analog 5. A1=PhAc, B1=2-(Carboxymethoxy)acetyl-RHI Monomer

### Step 1. A1=PhAc, B1=2-(carboxymethoxy)acetyl, B29=Boc-RHI

To dissolved Analog 1 (5.0 g, 0.844 mmol) in DMSO (50.0 mL) was added 2,2,6,6-tetramethylpiperidine (2.87 mL, 16.88 mmol) followed by dropwise addition of BOC-OSU (0.182 g, 0.844 mmol) in 1.0 mL of DMSO and stirred for 1 hr. Added to the same pot was diglycolic anhydride (0.093 g, 0.802 mmol) dissolved in a small amount of DMSO (˜1.0 mL). The reaction mixture was stirred for 1 h, quenched with ethanolamine, and stirred for 12 hrs to remove over-acylated material. The product was isolated by precipitation into IPAC-20% MTBE (1 L), then isolated the precipitate by filtration and dried on vacuum pump overnight UPLC-MS Method E, Rt=10.14 min, m/z=1536.97 [(M+1)/4].

### Step 2. Analog 5. A1=PhAc, B1=2-(carboxymethoxy)acetyl RHI Monomer

The material from Step 1 was treated with 50 mL of a mixture of TFA-water (5%)-iPr₃SiH (2.5%) over a period of 1 hr. The reaction mixture was added to 1.0 L of MTBE with stirring and cooling with ice. The precipitate was collected by filtration and washed with 500 mL of MTBE. The product was purified by prep. HPLC (Column Kromasil, C8 10 uM 100 A, size 250×50 mm; solvent A=water/0.05% TFA, solvent B=AcN/0.05% TFA), Flow=85 mL/min, gradient B in A 28-40% in 30 min. UPLC-MS Method C, Rt=3.66 min, m/z=1510.99 [(M+4)/4].

### General Method A: Dimerization of B1-Capped Analogs 2-5, Followed by Deprotection

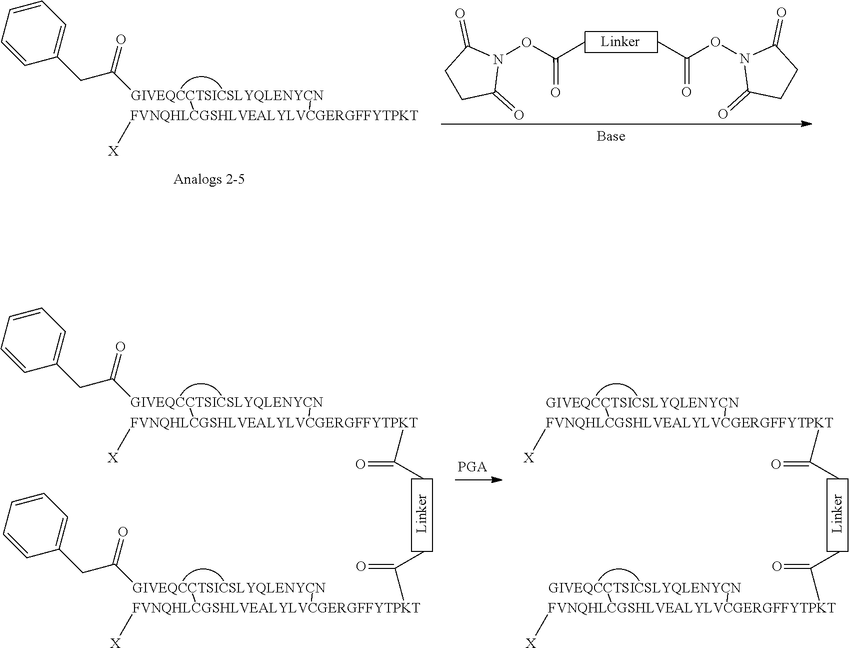

General Method A uses analogs 2-5, which contain a permanently installed capping group X on the B1 site and a phenylacetamide protective group on the A1 site. The phenylacetamide protective group is removable biocatalyticaly by the enzyme penicillin G acylase (PGA).

In the first step of the synthesis, the analog is dimerized using a linking reagent in the presence of organic base (triethylamine, Hunig's base, 2,2,6,6-tetramethylpiperidine, etc.) and organic solvent (DMSO, DMF). The resulting dimer is optionally precipitated by addition of the reaction mixture to diethyl ether, MTBA, IPAC, a mixture of MTBA and IPAC, or similar solvent. Optionally, the dimer can be isolated by reverse-phase preparative chromatography using acetonitrile-water mixture as a mobile phase with TFA as a modifier.

Alternatively, the crude reaction mixture containing the dimer can be diluted with water, pH-adjusted to pH 8-9, and used in the next step without isolation of the dimer.

In the second step of the synthesis, an aqueous solution of the dimer is treated with PGA in order to remove phenylacetamide protective groups. The enzyme is tolerant to the presence of up to v/v˜10% DMSO in the solution which may be the carry-over of solvent from the previous step. The optimal temperature range of the reaction is from room temperature to 30° C. The reaction time ranges from a few hours to 18 hrs (overnight). The product is isolated by reverse-phase preparative chromatography using acetonitrile-water mixture as a mobile phase with TFA as a modifier followed by lyophilization of chromatographic fractions.

The following examples were prepared using General Method A, modifying reagents and reaction conditions as necessary.

### Example 1

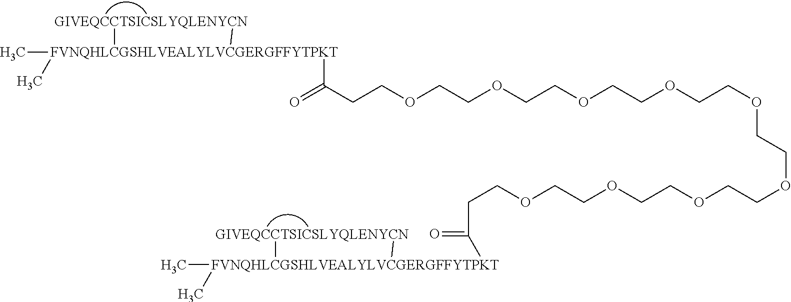

### Step 1. Dimerization

To dissolved Analog 2 (100 mg, 0.017 mmol) in DMSO (1.0 mL) was added triethylamine (0.094 mL, 0.673 mmol), followed by linker bis(2,5-dioxopyrrolidin-1-yl) 4,7,10,13,16,19,22,25,28-nonaoxahentriacontanedioate (5.97 mg, 8.42 umol) dissolved in 100 uL of DMSO and stirred for 1 hr. The reaction was quenched by addition of the reaction mixture into 10 mL of 20% AcN-water-0.05% TFA and adjusted pH to 2.5. The product was isolated by chromatography on C-8 phase (Column Kromasil, C8 10 uM 100 A, size 250×50 mm; solvent A=water/0.05% TFA, solvent B=AcN/0.05% TFA), Flow=85 mL/min, gradient B in A 27-38% in 30 min. Lyophilized to a solid. UPLC-MS Method C, Rt=3.85 min m/z=1770.3 (z=7)

### Step 2. Deprotection

The product of Step 1 was dissolved in 4.0 mL of water containing 30 mg of Na₂HPO₄ with pH adjusted to 8.5 and 10 mg of PGA was added. The mixture was gently shaken overnight at 30° C. The reaction mixture was diluted with 1.0 mL of AcN and acidified to pH 2.5 prior to injection on reverse-phase chromatographic column. The product was isolated by reverse-phase chromatography on C-8 phase (Column Kromasil, C8 10 uM 100 A, size 250×50 mm; solvent A=water/0.05% TFA, solvent B=AcN/0.05% TFA), Flow=85 mL/min, gradient B in A 27-38% in 30 min. Lyophilized to white solid. UPLC-MS Method C, Rt=3.59 min, m/z=1519.5 [(M+8)/8].

### Example 2

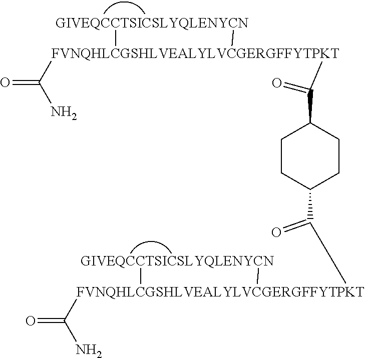

To the solution of Analog 3 (68 mg, 0.011 mmol) in 1.1 mL DMSO was added DIPEA (30 μl, 0.172 mmol) and stirred for 5 minutes followed by the linking reagent bis(2,5-dioxopyrrolidin-1-yl) (1R,R)-cyclohexane-1,4-dicarboxylate (2.05 mg, 5.60 μmol) pre-dissolved in 100 μl of anhydrous DMSO. The resulting mixture was stirred at room temperature for 90 minutes and added dropwise at 0° C. with pH monitoring to a centrifuge tube containing in 12 mL of water. During this addition, pH was kept below 9 with dropwise addition of 1N HCl.

In a separate vial, 20 mg of PGA was dissolved in 4.0 ml of water. The enzyme solution was added to the insulin derivative solution obtained in Step 1, and the pH of the final mixture was adjusted to 8.4, followed by shaking at 300 rpm and 30° C. for 17 h. The product was isolated by reverse-phase chromatography on C-8 phase (Column Kromasil, C8 10 uM 100 A, size 250×50 mm; solvent A=water/0.05% TFA, solvent B=AcN/0.05% TFA), Flow=85 mL/min, gradient B in A 31-37% in 30 min. Lyophilized to solid. UPLC-MS Method C, Rt=3.80 min, m/z=1692.1 [(M+7)/7].

### Example 3

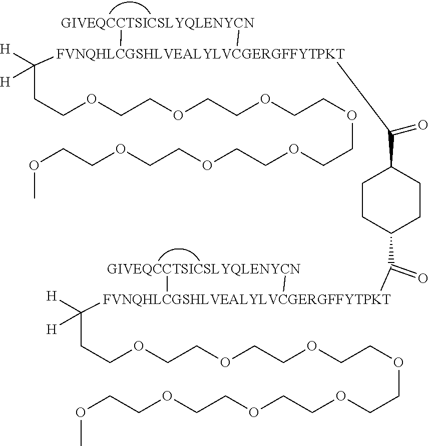

To a solution of Analog 4 (59 mg, 9.36 μmol) in DMSO (550 μl) was added 2,2,6,6-tetramethylpiperidine (63.2 μl, 0.374 mmol) followed by a solution of linking reagent bis(2,5-dioxopyrrolidin-1-yl) (1R,R)-cyclohexane-1,4-dicarboxylate ((1.714 mg, 4.68 umol)) pre-dissolved in 100 μl of anhydrous DMSO. The reaction mixture was stirred over 2 hrs and then added to 10 ml of water with ice cooling and maintained pH at 8.2. To this solution was added the enzyme PGA (10 mg) as solid. The reaction mixture was shaken in a stoppered vial overnight at 30° C. and acidified to pH 2.5 prior to purification. The product was purified by reverse-phase chromatography (Column Kromasil, C8 10 uM 100 A, size 250×50 mm; solvent A=water/0.05% TFA, solvent B=AcN/0.05% TFA), Flow=85 mL/min, gradient B in A 26-38% in 30 min. The product was isolated as a white powder following lyophilization of fractions, UPLC-MS Method C, Rt=3.64 min, m/z=1565.9 [(M+8)/8].

### Example 4

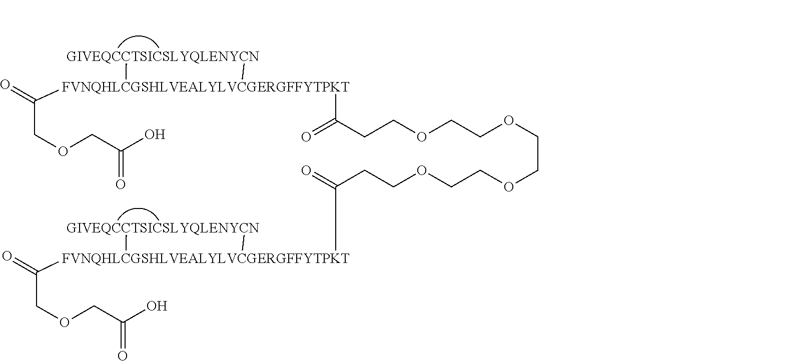

To a solution of Analog 5 (130 mg, 0.022 mmol) in 1.3 mL of DMSO was added 2,2,6,6-tetramethylpiperidine (36.3 μl, 0.215 mmol) followed by the linking reagent bis(2,5-dioxopyrrolidin-1-yl)4,7,10,13-tetraoxahexadecanedioate (5.25 mg, 10.76 umol) dissolved in a 100 μL of DMSO and stirred for 1 hr. The reaction mixture was added to 10.0 ml of water which was cooled with ice and adjusted pH using AcOH and NaOH as needed to pH=8.2. PGA enzyme (10 mg) was added and the reaction gently shaken at 30° C. overnight. The product was purified by reverse-phase chromatography on C-8 phase (Column Kromasil, C8 10 uM 100 A, size 250×50 mm; solvent A=water/0.05% TFA, solvent B=AcN/0.05% TFA), Flow=85 mL/min, gradient B in A 27-38% in 30 min. UPLC-MS Method D, Rt=3.96 min, m/z=1729.9 [(M+7)/7].

TABLE IV
The following compounds were obtained using the procedure analogous to General Method A
but substituting appropriate starting materials that are either commercially available or prepared
using procedure analogous to those described in Examples 1 through 4.

| | | Retetion |
|---|---|---|
| | | time, |
| Ex- | | Observed |
| am- | | m/z, |
| ple | | UPLC |
| # | | Method |
| 5 | 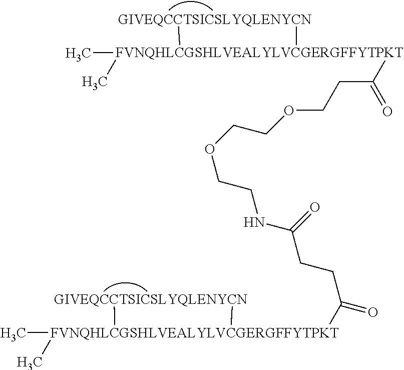 | UPLC Method C, Rt = 3.54 min, m/z = 1701.6 (z = 7) |
| 6 | 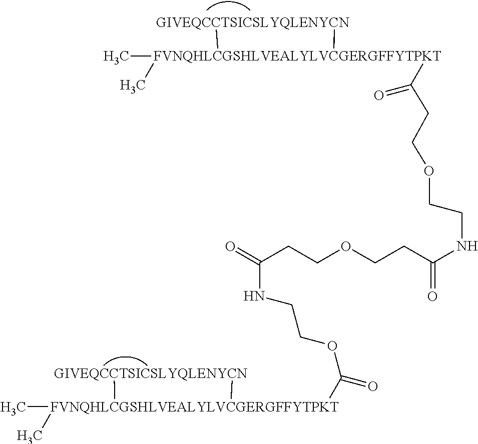 | UPLC Method C, Rt = 3.63 min, m/z = 1719.9 (z = 7) |
| 7 | 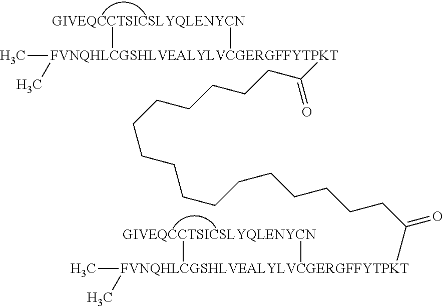 | UPLC Method A, Rt = 0.95 min, m/z = 1706.9 (z = 7) |
| 8 | 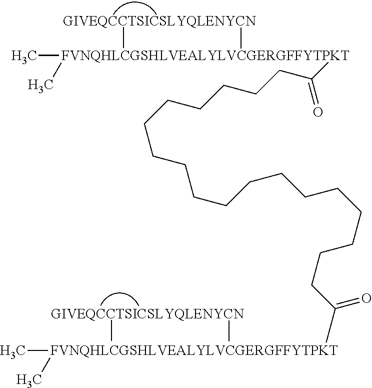 | UPLC Method C, Rt = 0.98 min, m/z = 1497.9 (z = 8) |
| 9 | 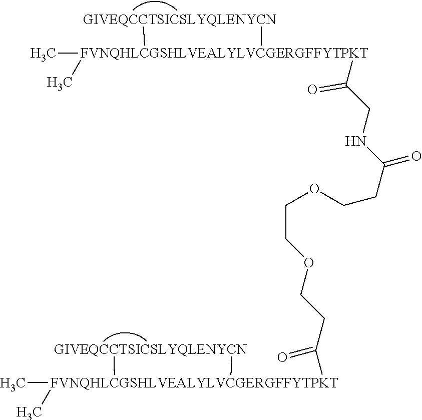 | UPLC Method A, Rt = 0.86 min, m/z = 1488.1 (z = 8) |
| 10 | 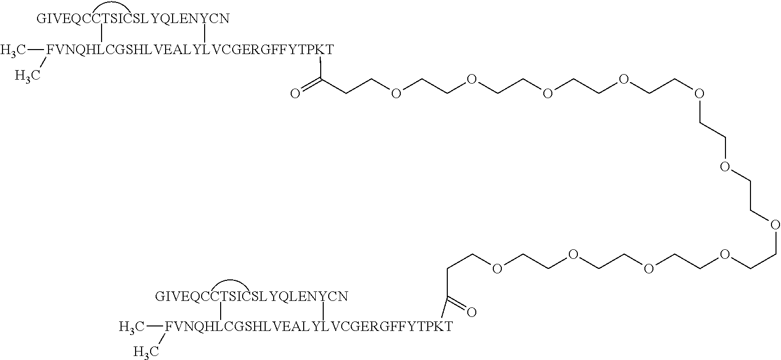 | UPLC Method C, Rt = 3.58 min, m/z = 1749.2 (z = 7) |
| 11 | 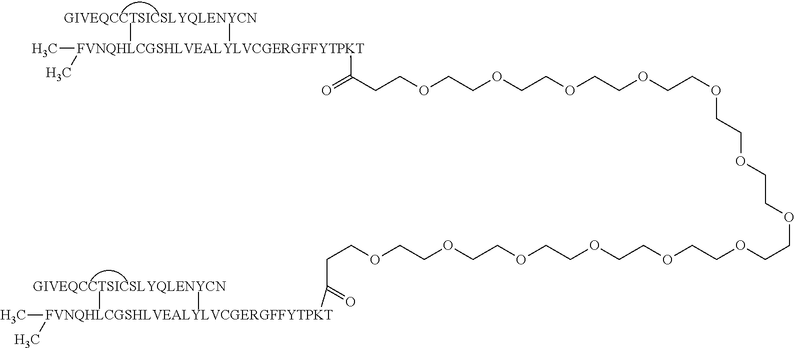 | UPLC Method C, Rt = 3.54 min, m/z = 1541.6 (z = 8) |
| 12 | 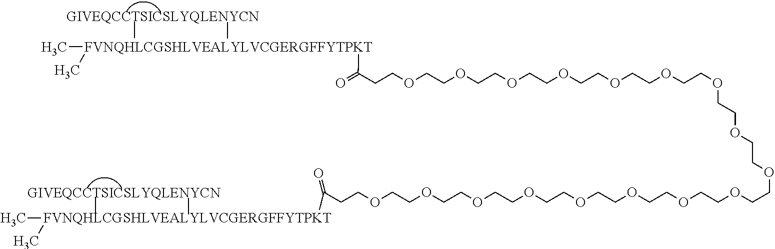 | UPLC Method C, Rt = 3.21 min, m/z = 1786.8 (z = 7) |
| 13 | 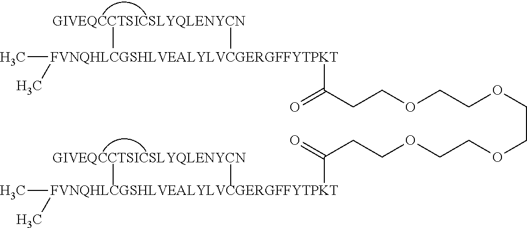 | UPLC Method A, Rt = 0.86 min, m/z = 1705.0 (z = 7) |
| 14 | 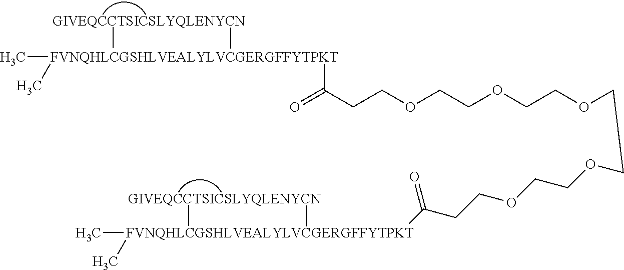 | UPLC Method A, Rt = 0.86 min, m/z = 1711.5 (z = 7) |
| 15 | 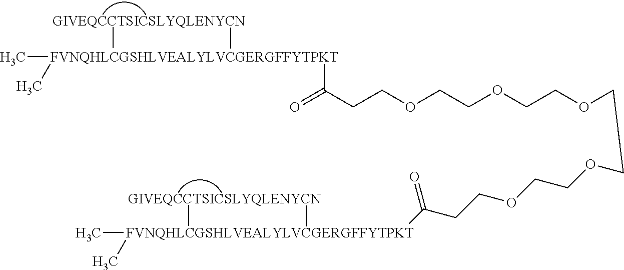 | UPLC Method D, Rt = 2.78 min, m/z = 1508.7 (z = 8) |
| 16 | 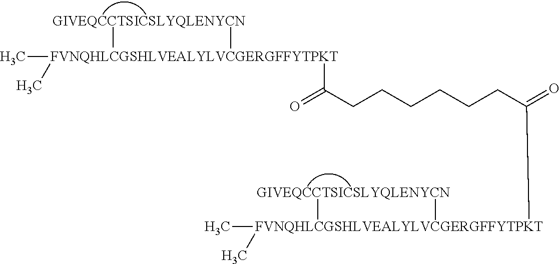 | UPLC Method C, Rt = 3.66 min, m/z = 1477.3 (z = 8) |
| 17 | 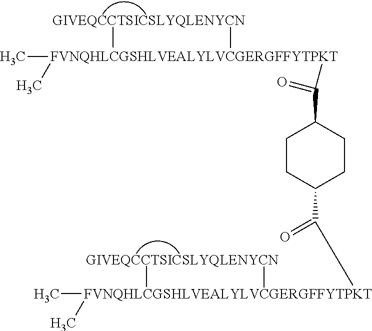 | UPLC Method A, Rt = 0.95 min, m/z = 1706.9 (z = 7) |
| 18 | 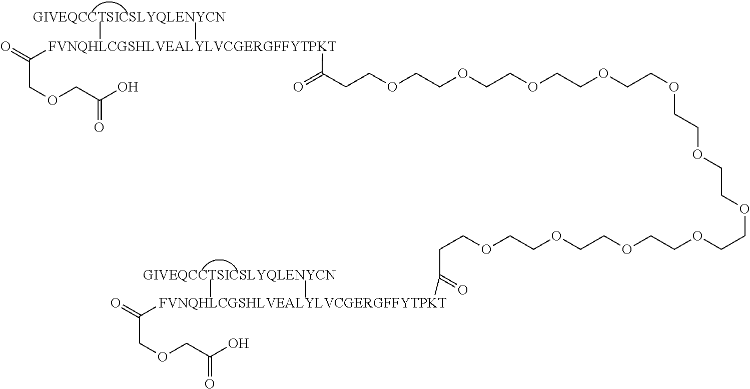 | UPLC Method D, Rt = 3.70 min, m/z = 1744.4 (z = 7) |
| 19 | 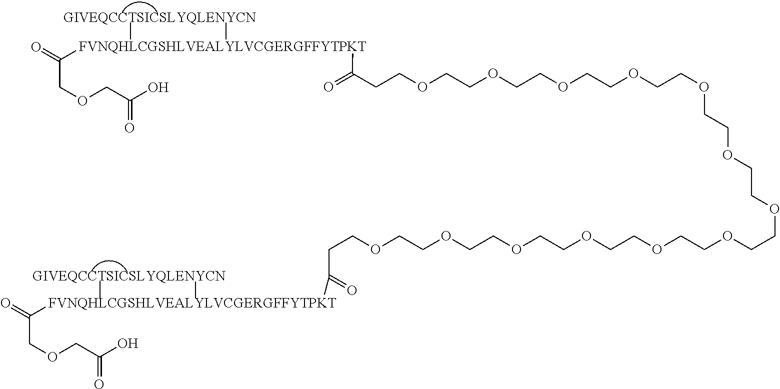 | UPLC Method D Rt = 3.71 min, m/z = 1563.8 (z = 8) |
| 20 | 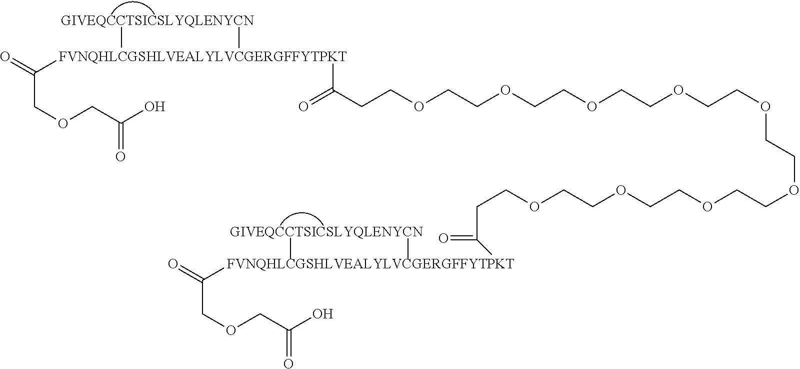 | UPLC Method D Rt = 3.74 min, m/z = 1541 (z = 8) |
| 21 | 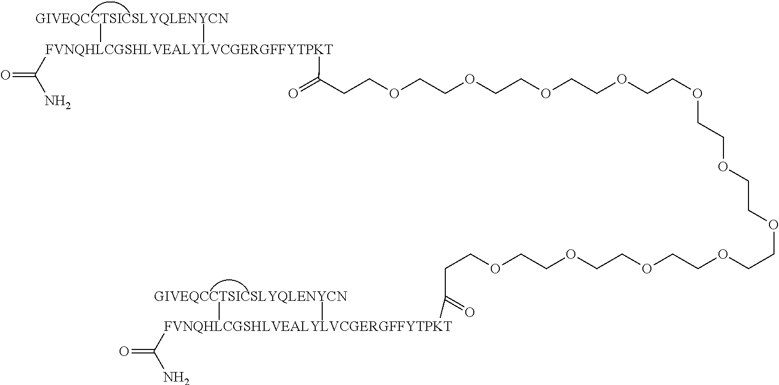 | UPLC Method C Rt = 3.41 min, m/z = 1753.4 (z = 7) |
| 22 | 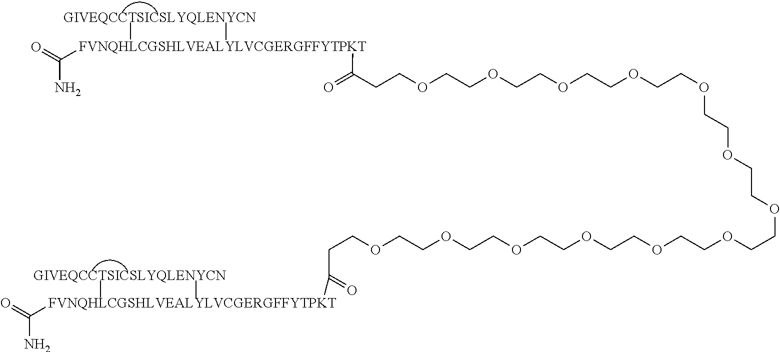 | UPLC Method C Rt = 3.80 min, m/z = 1766.5 (z = 7) |
| 23 | 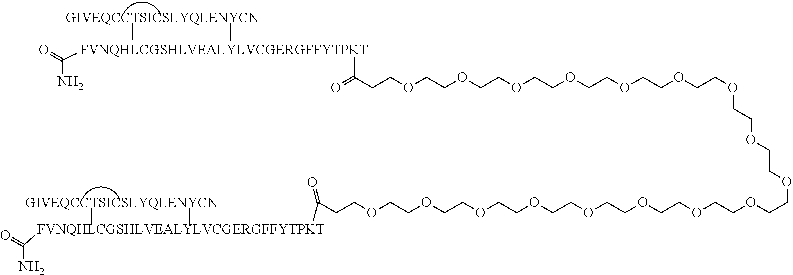 | UPLC Method C, Rt = 3.94 min, m/z = 1791.28 (z = 7) |
| 24 | 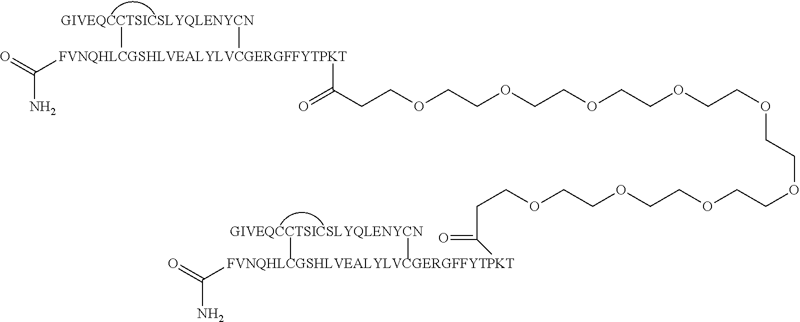 | UPLC Method C, Rt = 3.96 min, m/z = 1740.9 (z = 7) |
| 25 | 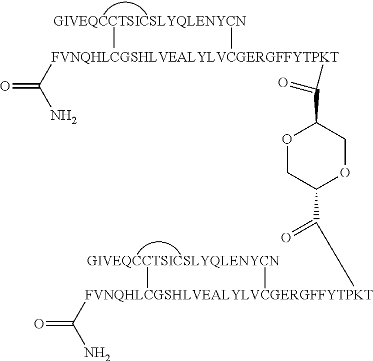 | UPLC Method C, Rt = 3.69 min, m/z = 1481.1 (z = 7) |

### General Method B: Dimerization of Analog 1, Followed by B1,B1′-Capping, Followed by Deprotection

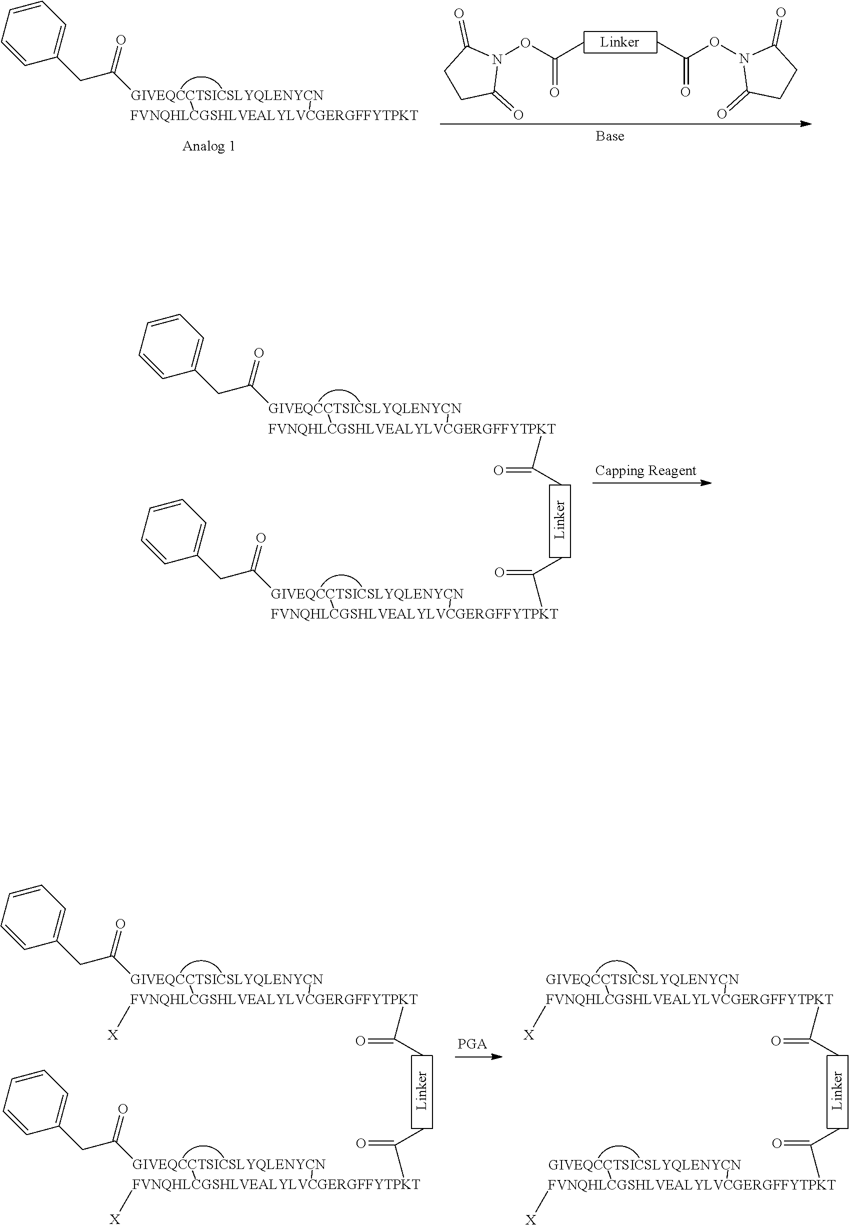

Analog 1 serves as the starting material for the process. In the first step of the synthesis, the Analog 1 is dimerized using a linking reagent in the presence of organic base (triethylamine, Hunig's base, 2,2,6,6-tetramethylpiperidine, etc.) and organic solvent (DMSO, DMF). The resulting dimer is optionally precipitated by addition of the reaction mixture to diethyl ether, MTBA, IPAC, a mixture of MTBA and IPAC, or similar solvent. Optionally, the dimer can be isolated by reverse-phase preparative chromatography using acetonitrile-water mixture as a mobile phase with TFA as a modifier. Alternatively, the resulting dimer is not isolated, but instead is treated in the same pot with a capping reagent to cap B1,B1′ sites of the dimer.

The resulting B1,B1′-capped dimer is optionally precipitated by addition of the reaction mixture to diethyl ether, MTBA, IPAC, a mixture of MTBA and IPAC, or similar solvent. Optionally, the dimer can be isolated by reverse-phase preparative chromatography using acetonitrile-water mixture as a mobile phase with TFA as a modifier. Alternatively, the crude reaction mixture containing the B1,B1′-capped dimer can be diluted with water, pH-adjusted to pH in the interval of 8-9, and used in the last step without isolation of the dimer.

In the last step of the synthesis, an aqueous solution of the dimer is treated with PGA in order to remove phenylacetamide protective groups. The enzyme is tolerant to the presence of up to v/v ˜10% DMSO in the solution which may be the carry-over of solvent from the previous step. The optimal temperature range of the reaction is from room temperature to 30° C. The reaction time ranges from a few hours to 18 hrs (overnight). The product is isolated by reverse-phase preparative chromatography using acetonitrile-water mixture as a mobile phase with TFA as a modifier followed by lyophilization of chromatographic fractions.

### Example 26

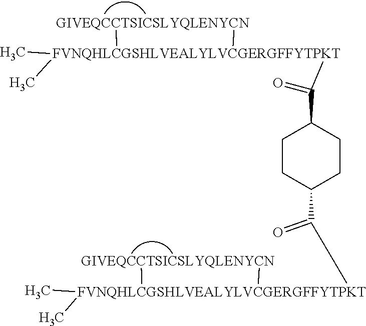

### Step 1. Dimerization

To the solution of Analog 1 (1.0 g, 0.140 mmol) in 5.5 mL DMSO was added 2,2,6,6-tetramethylpiperidine (332 μL, 1.967 mmol) and the reaction mixture was stirred for 5 minutes. To this solution was added solution of bis(2,5-dioxopyrrolidin-1-yl) (2R,5S)-1,4-dioxane-2,5-dicarboxylate (26 mg, 0.070 mmol) in 500 μl of anhydrous DMSO and the resulting mixture was stirred at room temperature for 45 minutes. The crude reaction mixture was added dropwise to a 100 mL flask containing 75 mL of IPAc/MTBE (4:1). The resulting white suspension was filtered and rinsed with (3×10 mL of IPAc), dried under high vacuum and used in the following step without further purification. UPLC-MS Method C, Rt=3.77 min, m/z=1499 [(M+8)/8].

### Step 2. Capping at B1 Site by Reduction Amination

To a suspension of material of step 1 (324 mg, 0.020 mmol) in water (6 mL) was added acetic acid dropwise until reaction mixture is in fully solution (pH=2.8). Added 37% aqueous solution of formaldehyde (8 μl, 0.107 mmol) followed by a solution of sodium cyanoborohydride (7 mg, 0.111 mmol) in 0.1 mL H₂O and the resulting suspension were stirred for 30 minutes. The reaction mixture was quenched with ethanolamine (15 μl, 0.248 mmol) and the pH of the reaction crude was adjusted to pH 2. Diafiltrated in 3K Amicon Centrifuge tube (2×10 mL) to volume of ˜10 mL. The material was used in the following step without purification. UPLC-MS Method A, Rt=0.90 min, m/z=1507.03 [(M+8)/8].

### Step 3: Deprotection

The intermediate from previous step was diluted with 20 mL of H₂O (total volume ˜30 mL) and the pH of the resulting mixture was adjusted to 8.3 by dropwise addition of 1N NaOH. In a separate dissolved ˜60 mg of PGA was dissolved in 10 mL of H₂O and the enzyme solution was added to the insulin derivative solution. The mixture was shaken at 300 rpm and 30° C. for 74 h. The product was isolated by chromatography (Column Kromasil, C8 10 uM 100 A, size 250×50 mm; solvent A=water/0.05% TFA, solvent B=AcN/0.05% TFA), Flow=85 mL/min, gradient B in A 27-38% in 30 min. UPLC-MS Method A, Rt=0.86 min, m/z=1477.46 [(M+8)/8].

### Example 27

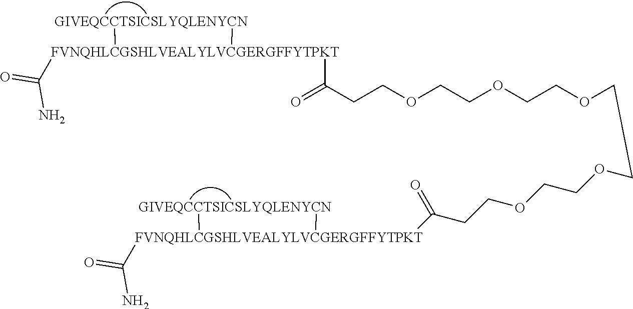

### Step 1. Dimerization

To dissolved Analog 1 (500 mg, 0.084 mmol) in DMSO (5 mL) was added triethylamine (0.353mL, 2.53 mmol) followed by the linking reagent bis (2,5-dioxopyrrolidin-1-yl) 4,7,10,13,16-pentaoxanonadecanedioate (20.22 mg, 0.038 mmol) dissolved in 100 uL of DMSO. After 1 hr of stirring the reaction was analyzed and displayed dimerization product. UPLC-MS Method C, Rt=3.76 min, m/z=1520.89 [(M+8)/8]. The material was used as the DMSO solution without further isolation.

### Step 2. Capping B1,B1′ Sites with Carbamoyl

Half of the solution containing the material of Step 1 (2.5 mL of solution) was diluted with water (50 mL) containing potassium phosphate dibasic (59.1 mg, 0.339 mmol) and the pH was adjusted to 7.4 with 1M HCl. Potassium cyanate (133 mg, 1.646 mmol) was added and stirred over 36 hrs. The product was isolated by reverse-phase chromatography on C-8 phase (Column Kromasil, C8 10 uM 100 A, size 250×50 mm; solvent A=water/0.05% TFA, solvent B=AcN/0.05% TFA), Flow=85 mL/min, gradient B in A 28-38% in 30 min. UPLC-MS Method F, Rt=3.76 min, m/z=1530.95 [(M+8)/8].

### Step 3. Deprotection of PhAc Protective Groups

To the material of Step 2 was dissolved in 6.0 mL of water, pH adjusted to 8, was added 30 mg of enzyme PGA with continued shaking at 35° C. After 6 hrs the reaction was complete and the product was isolated by reverse-phase chromatography (Column Kromasil, C8 10 uM 100 A, size 250×50 mm; solvent A=water/0.05% TFA, solvent B=AcN/0.05% TFA), Flow=85 mL/min, gradient B in A 26-34% in 30 min. UPLC-MS Method C, Rt=3.70 min, m/z=1501.37 [(M+8)/8].

### Example 28

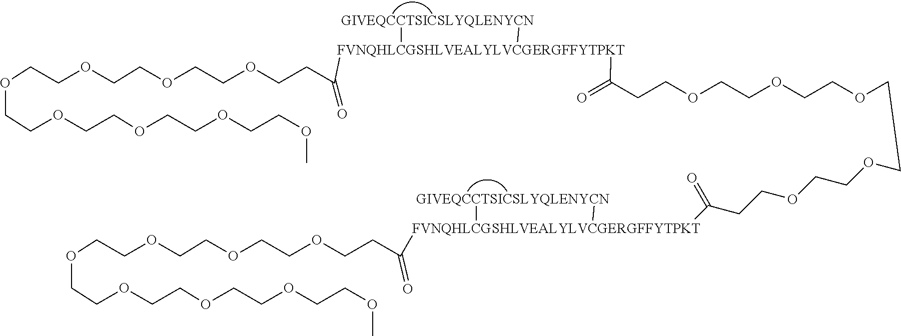

### Step 1. Dimerization

To dissolved Analog 1 (500 mg, 0.084 mmol) in DMSO (5 mL) was added triethylamine (0.353 mL, 2.53 mmol), followed by the linking reagent bis(2,5-dioxopyrrolidin-1-yl) 4,7,10,13,16-pentaoxanonadecanedioate (20.22 mg, 0.038 mmol) dissolved in 100 uL of DMSO. After 1 hr of stirring, the reaction was analyzed and displayed dimerization product. UPLC-MS Method C, Rt=3.76 min, m/z=1520.89 [(M+8)/8]. The material was used as DMSO solution without isolation.

### Step 2. Capping on B1,B1′ Sites

Half of the solution containing the material of Step 1 (˜2.5 mL) was treated with triethylamine (0.057 mL, 0.411 mmol) and a solution of 2,5-dioxopyrrolidin-1-yl 2,5,8,11,14,17,20,23-octaoxahexacosan-26-oate (12.58 mg, 0.025 mmol) in 100 uL of DMSO and stirred overnight. The product was isolated by reverse-phase chromatography on C-8 phase (Column Kromasil, C8 10 uM 100 A, size 250×50 mm; solvent A=water/0.05% TFA, solvent B=AcN/0.05% TFA), Flow=85 mL/min, gradient B in A 29-39% in 30 min (Gilson A). UPLC-MS Method F, Rt=3.78 min, m/z=1439.04 [(Z+9)/9].

### Step 3. Deprotection

The product of step 2 (70 mg) was dissolved in 5.0 mL of water containing 25 mg of Na2HPO4, and adjusted pH to 8.2. The 10 mg of enzyme PGA was added and the shaking was continued at 30° C. overnight. The product was isolated by reverse-phase chromatography (Column Kromasil, C8 10 uM 100 A, size 250×50 mm; solvent A=water/0.05% TFA, solvent B=AcN/0.05% TFA), Flow=85 mL/min, gradient B in A 26-36% in 30 min. UPLC-MS Method C, Rt=3.79 min, m/z=1589.10 [(Z+8)/8].

### Example 29

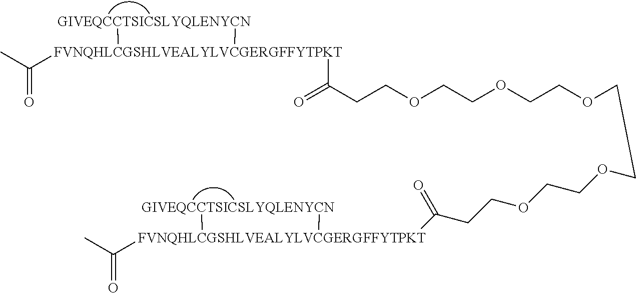

### Step 1. Dimerization

To a solution of Analog 1 (560 mg, 0.089 mmol) in DMSO (2.5 mL) was added 2,2,6,6-tetramethylpiperidine (0.225 mL, 1.333 mmol). Bis(2,5-dioxopyrrolidin-1-yl) 4,7,10,13,16-pentaoxanonadecane-1,19-dioate (18.92 mg, 0.036 mmol) dissolved in 100 μL of DMSO was added. The mixture was allowed to stir for 4 hours, and the reaction mixture was added to 100 mL of IPAC/MTBE mixture (4/1). The formed precipitate was collected by filtration and dried under vacuum overnight. UPLC-MS Method C, Rt=3.65 min, m/z=1519.96 [(Z+8)/8].

### Step 2. Capping of B1,B1′ Sites and Deprotection

To a solution of Step 1 (1.0 g, 0.082 mmol) in DMSO (2.5 mL) was added TEA (0.172 mL, 1.234 mmol). The mixture was treated with a solution of 2,5-dioxopyrrolidin-1-yl acetate (0.039 g, 0.247 mmol) in DMSO (500 μL) and stirring continued for 4 hrs, followed by addition into 100 mL of a mixture of IPAC/MTBE (4/1). The precipitate was collected by filtration, dried under vacuum overnight, dissolved in 48 mL of water, adjusted to pH 8.8, and the solution was treated with a solution of PGA (120 mg) in water (12 mL). Shaking at 300 rpm was continued at 30° C. overnight. The product was isolated by reverse-phase chromatography (Column Kromasil, C8 10 uM 100 A, size 250×50 mm; solvent A=water/0.05% TFA, solvent B=AcN/0.05% TFA) and obtained as a solid after lyophilization of fractions. UPLC-MS Method C, Rt=3.63 min, m/z=1715.54 [(Z+7)/7].

### Example 30

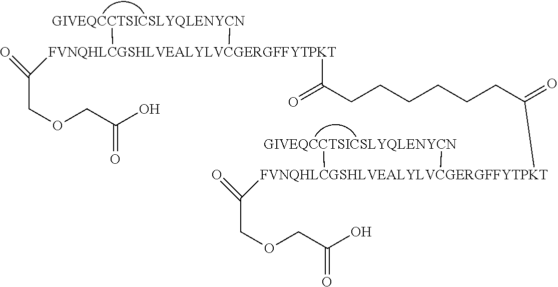

### Step 1. Dimerization

To a solution of Analog 1 (1000 mg, 0.169 mmol) in DMSO (10 mL) was added 2,2,6,6-tetramethylpiperidine (715 mg, 5.06 mmol) followed by the linker disuccinimidyl suberate (31.1 mg, 0.084 mmol) and stirred for 1 hr. The reaction mixture was added to a stirring mixture of IPAC/Et2O (4:1, 500 mL) and the precipitate was collected by filtration and dried on filter under nitrogen overnight. The product was isolated by reverse-phase chromatography on C-8 phase (Column Kromasil, C8 10 uM 100 A, size 250×50 mm; solvent A=water/0.05% TFA, solvent B=AcN/0.05% TFA), Flow=85 mL/min, gradient B in A 26-40% in 30 min. UPLC-MS Method D, Rt=1.48 min, m/z=1199.96 [(Z+10)/10].

### Step 2. Capping of B1,B1′ Sites and Deprotection

To a mixture of the product of Step 1 (151 mg, 0.013 mmol) and triethylamine (0.070 mL, 0.504mmol)) in DMSO (1.5 mL) was added diglycolic anhydride (3.65 mg, 0.031 mmol) dissolved in a small amount of DMSO (75 uL). After 3 hrs, the mixture was diluted with water (10 mL) along with cooling, and the pH adjusted to 8.1 with 1M HCl. PGA (10 mg) was added. The mixture was shaken at room temperature overnight. The product was purified by reverse-phase chromatography on C-8 phase (Column Kromasil, C8 10 uM 100 A, size 250×50 mm; solvent A=water/0.05% TFA, solvent B=AcN/0.05% TFA), Flow=85 mL/min, gradient B in A 26-36% in 30 min. UPLC-MS Method D, Rt=1.48 min, m/z=1499.1 [(Z+8)/8].

TABLE V
The following compounds were obtained using the procedure analogous to General Method B
but substituting appropriate starting materials that are either commercially available or prepared
using procedure analogous to those described in Examples 26 through 30. Many of the fonts in
the table below are likely to small

| | | Retetion |
|---|---|---|
| | | time, |
| Ex- | | Observed |
| am- | | m/z, |
| ple | | UPLC |
| # | | Method |
| 31 | 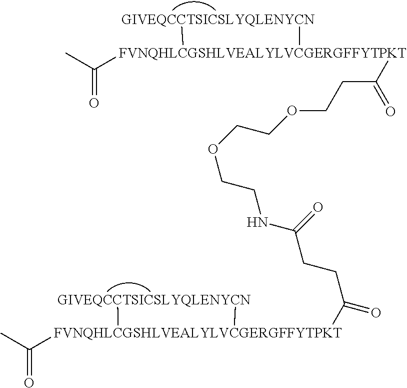 | UPLC Method C, Rt = 3.70 min, m/z = 1493.4 (z = 8) |
| 32 | 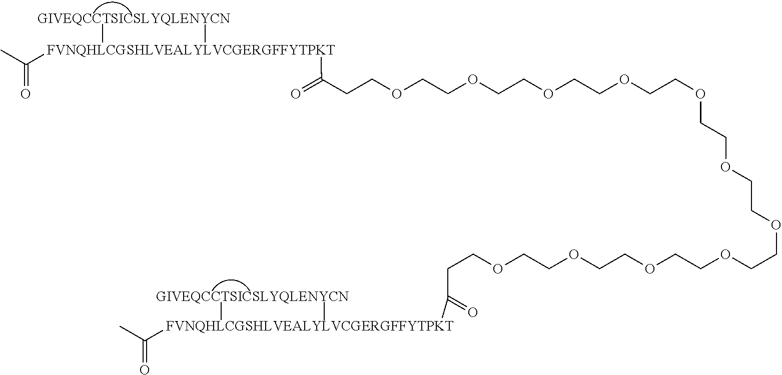 | UPLC Method C, Rt = 3.64 min, m/z = 1753.1 (z = 7) |
| 33 | 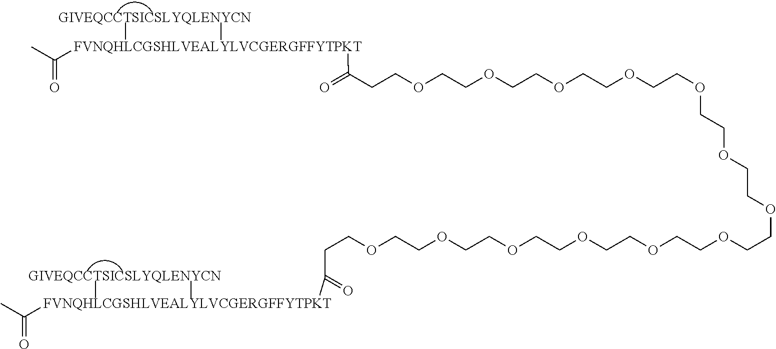 | UPLC Method C, Rt = 3.87 min, m/z = 1765.7 (z = 7) |
| 34 | 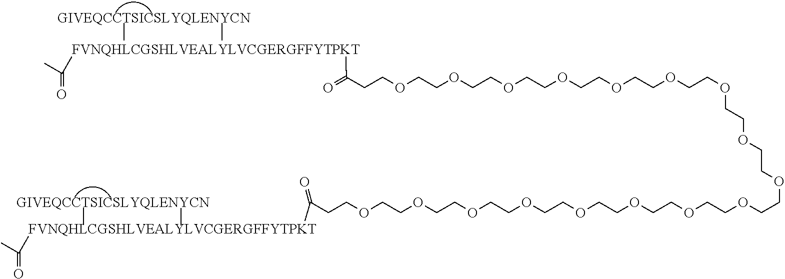 | UPLC Method C, Rt = 3.29 min, m/z = 1792.4 (z = 7) |
| 35 | 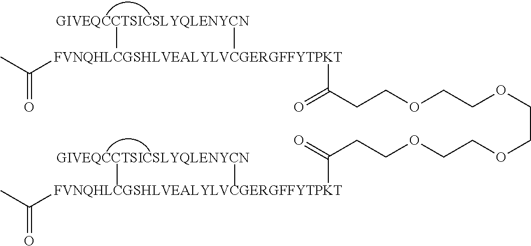 | UPLC Method A, Rt = 0.89 min, m/z = 1808.1 (z = 7) |
| 36 | 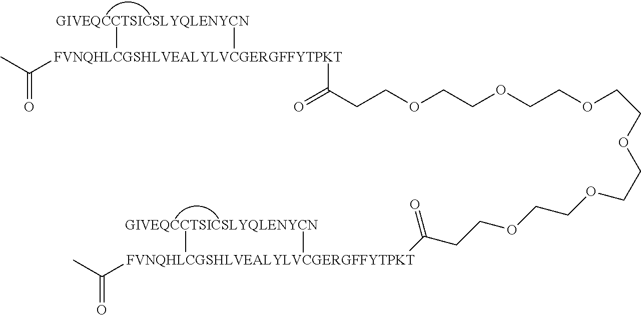 | UPLC Method C, Rt = 3.85 min, m/z = 1721.7 (z = 7) |
| 37 | 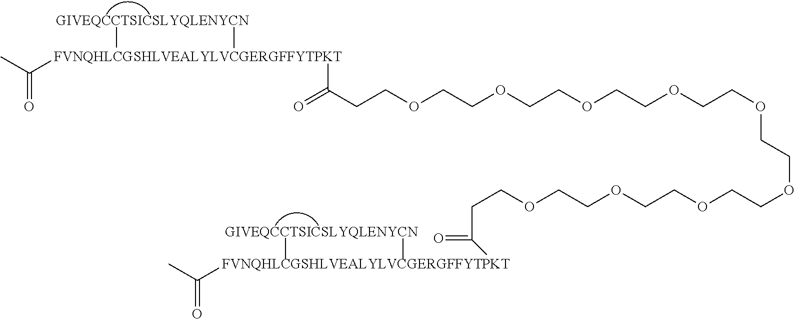 | UPLC Method C, Rt = 3.23 min, m/z = 1740.5 (z = 7) |
| 38 | 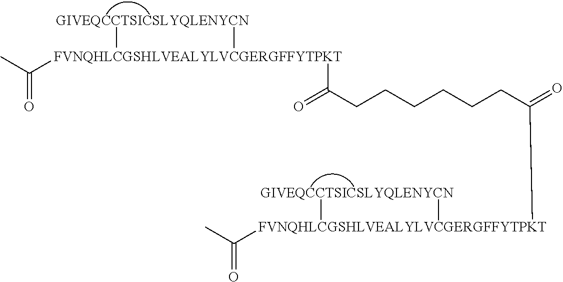 | UPLC Method C, Rt = 1.46 min, m/z = 1692.1 (z = 7) |
| 39 | 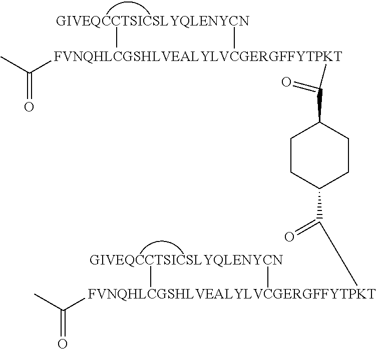 | UPLC Method C, Rt = 3.81 min, m/z = 1691.7 (z = 7) |
| 40 | 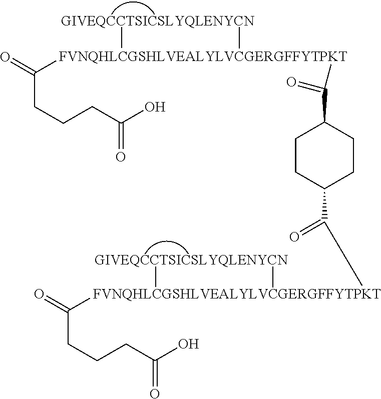 | UPLC Method C, Rt = 3.60 min, m/z = 1712.0 (z = 7) |
| 41 | 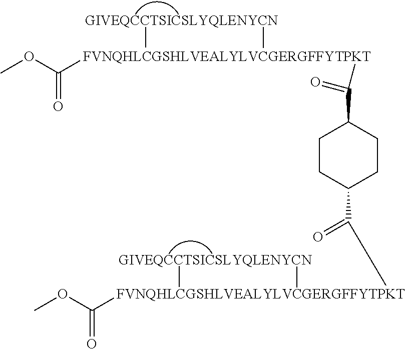 | UPLC Method C, Rt = 3.88 min, m/z = 1700.3 (z = 7) |
| 42 | 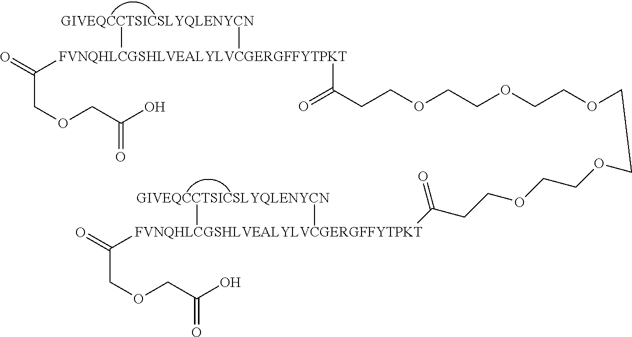 | UPLC Method C, Rt = 3.79 min, m/z = 1520.7 (z = 8) |
| 43 | 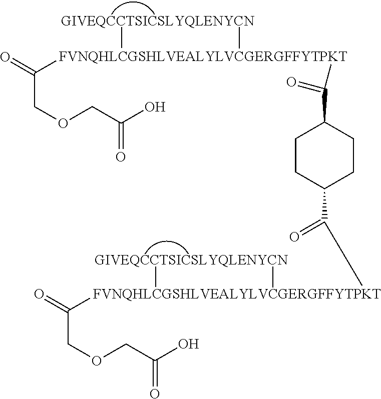 | UPLC Method C, Rt = 3.81 min, m/z = 1712.8 (z = 7) |

### Example 44

### A. Insulin Receptor Binding Assays were Performed as Follows

IR binding assay was run in a scintillation proximity assay (SPA) in 384-well format using cell membranes prepared from CHO cells overexpressing human IR (B) grown in F12 media containing 10% FBS and antibiotics (G418, Penicillin/Strepavidin). Cell membranes were prepared in 50 mM Tris buffer, pH 7.8 containing 5 mM MgCl₂. The assay buffer contained 50 mM Tris buffer, pH 7.5, 150 mM NaCl, 1 mM CaCl₂, 5 mM MgCl₂, 0.1% BSA and protease inhibitors (Complete-Mini-Roche). Cell membranes were added to WGA PVT PEI SPA beads (5 mg/mL final concentration) followed by addition of insulin dimer molecules at appropriate concentrations. After 5-15 min incubation at room temperature, ¹²⁵[I]-insulin was added at 0.015 nM final concentration for a final total volume of 50 μL. The mixture was incubated with shaking at room temperature for 1 to 12 hours followed by scintillation counting to determine ¹²⁵[I]-insulin binding to IR and the titration effects of insulin dimer molecules on this interaction.

### B. Insulin Receptor (IR) AKT-Phosphorylation Assays were Performed as Follows

Insulin receptor activation can be assessed by measuring phosphorylation of the Akt protein, a key step in the insulin receptor signaling cascade. CHO cell lines overexpressing human IR were utilized in an HTRF sandwich ELISA assay kit (Cisbio “Phospho-AKT (Ser473) and Phospho-AKT (Thr308) Cellular Assay Kits”). Cells were grown in F12 media supplemented with 10% FBS, 400 μg/mL G418 and 10 mM HEPES. Prior to assay, the cells were incubated in serum free media for 2 to 4 hr. Alternatively, the cells could be frozen and aliquoted ahead of time in media containing 20% DMSO and used in the assay upon thawing, spin down and re-suspension. Cells were plated at 10,000 cells per well in 20 μL of the serum free F12 media in 384-well plates. Humulin and insulin glargine controls were run on each plate of test compounds. The titrated compounds were added to the cells (2 μL per well, final concentrations=1000 nM titrated down to 0.512 pM in 1:5 fold dilutions) and incubated at 37° C. for 30 min. The cells were lysed with 8 μL of the prepared lysis buffer provided in the CisBio kit and incubated at 25° C. for 1 hr. The diluted antibody reagents (anti-AKT-d2 and anti-pAKT-Eu3/cryptate) were prepared according to the kit instructions and then 10 μL was added to each well of cell lysate followed by incubation at 25° C. for 3.5 to 5 hr. The plate was read by in an Envision plate reader (Excitation=320 nm; Emission=665 nm) to determine the IR pAkt agonist activity with regard to both potency and maximum response for each compound.

Table VI shows the in vitro biological activity of the insulin dimers towards the insulin receptor (IR). The activities were measured by either ligand competition assays as described in Example 44 A or functional AKT-phosphorylation assay as described in Example 44 B.

TABLE VI

| | IR | IR pAkt | IR |
|---|---|---|---|
| Dimer | Binding | EC₅₀ | pAkt |
| No. | IC₅₀ (nM) | (nM) | % Max |
| 1 | 3.76 | 0.82 | 44 |
| 2 | 2.05 | 0.28 | 25 |
| 3 | 1.17 | 0.16 | 49 |
| 4 | 2.02 | 0.04 | 22 |
| 5 | 0.86 | 0.09 | 26 |
| 6 | 1.81 | 0.15 | 22 |
| 7 | 21.98 | 1.26 | 45 |
| 8 | 25.96 | 1.82 | 39 |
| 9 | 0.01 | 0.04 | 24 |
| 10 | 6.69 | 0.30 | 36 |
| 11 | 49.66 | 1.72 | 63 |
| 12 | 5.13 | 0.13 | 57 |
| 13 | 0.81 | 0.08 | 26 |
| 14 | 1.45 | 0.06 | 28 |
| 15 | 21.29 | 0.78 | 33 |
| 16 | 1.83 | 0.18 | 37 |
| 17 | 2.15 | 0.07 | 31 |
| 18 | 3.75 | 0.06 | 40 |
| 19 | 4.62 | 0.14 | 50 |
| 20 | 2.99 | 0.12 | 40 |
| 21 | 5.91 | 0.61 | 58 |
| 22 | 5.26 | 0.25 | 49 |
| 23 | 0.69 | 0.24 | 62 |
| 24 | 0.45 | 0.85 | 52 |
| 25 | 3.66 | 0.11 | 30 |
| 26 | 1.53 | 0.06 | 30 |
| 27 | 0.85 | 0.08 | 35 |
| 28 | 2.85 | 0.15 | 39 |
| 29 | 0.96 | 0.04 | 34 |
| 30 | 1.93 | 0.04 | 31 |
| 31 | 0.69 | 0.28 | 58 |
| 32 | 2.51 | 0.15 | 47 |
| 33 | 27.89 | 2.90 | 80 |
| 34 | 4.12 | 0.15 | 54 |
| 35 | 0.32 | 0.09 | 22 |
| 36 | 1.15 | 0.10 | 27 |
| 37 | 3.70 | 0.22 | 29 |
| 38 | 0.32 | 0.02 | 29 |
| 39 | 2.09 | 0.66 | 44 |
| 40 | 7.12 | 0.29 | 40 |
| 41 | 1.03 | 0.13 | 39 |
| 42 | 0.97 | 0.09 | 30 |
| 43 | 0.33 | 0.10 | 30 |

### Example 45

Improved chemical and biophysical stability of B1,B1′-capped insulin dimers:

Capping of B1 and B1′ terminals of the insulin dimers unexpectedly improves chemical and biophysical stability of these compounds, as evident from Table VII. For the stability studies, the compounds were dissolved at concentrations of 20 mg/mL in a buffer containing 7 mM sodium phosphate buffer, pH 7.4, containing 16 mg/mL glycerin, 2.0 mg/mL m-cresol, 1.5 mg/mL phenol, and ZnCl₂ added at 0.671 eq/molar ratio. Chemical degradation and formation of high molecular weight (HMW) aggregates was followed over a period of 4 weeks, typically under stress-test temperature of 40° C. Chemical degradation was measured by HPLC and expressed as purity loss in the table. Formation of HMW aggregates was measured by size exclusion chromatography. Insulin dimers lacking the capping groups at B1 and B1′ terminals were included in the studies as reference compound. Some of these reference compounds showed loss of purity and formation of HMW during 4 week storage even at a low temperature of 5° C. On the other hand. insulin dimers with the capping groups on B1 and B1′ sites showed protection from purity loss and formation of HMW aggregates.

TABLE VII

| | | | | HMW Aggregate |
|---|---|---|---|---|
| | Linker | B1, B1′ Capping | Purity Loss, % | Formation, % |
| Dimer# | Name | Group | (4 Weeks@40° C.) | (4 Weeks@40° C.) |
| Reference | PEG4 | None | 8.27 | 3.56 |
| 4 | PEG4 | 2-(carboxymethoxy) | 0.38 | 0.08 |
| | | acetyl | | |
| 13 | PEG4 | Me2 | 4.25 | 1.48 |
| 35 | PEG4 | Ac | 2.83 | 0.25 |
| Reference | PEG5 | None | 13.4 | 9.4 |
| | | | (4 Weeks@5° C.) | (4 weeks@5° C.) |
| 14 | PEG5 | Me2 | 5.8 | 1.22 |
| 27 | PEG5 | carbamoyl | 2.04 | 0.44 |
| 42 | PEG5 | 2-(carboxymethoxy) | 0.59 | 0.12 |
| | | acetyl | | |
| Reference | PEG17 | None | 17.2 | 8.5 |
| | | | (4 Weeks@5° C.) | (4 Weeks@5° C.) |
| 12 | PEG17 | Me2 | 10 | 0.5 |
| 23 | PEG17 | Carbamoyl | 0.5 | 0.5 |
| 34 | PEG17 | Ac | 4.9 | 0.2 |
| Reference | Suberic | None | 12.68 | 5.59 |
| 16 | Suberic | Me2 | 8.52 | 4.77 |
| 30 | Suberic | 2-(carboxymethoxy) | 1.89 | 0.7 |
| | | acetyl | | |
| 38 | Suberic | Ac | 2.81 | 1.04 |
| Reference | Trans- | None | 8 | 10 |
| | 1,4- | | | |
| | dioxane- | | | |
| | 2,5- | | | |
| | diacyl | | | |
| 25 | Trans- | Carbamoyl | 1.5 | 3.5 |
| | 1,4- | | | |
| | dioxane- | | | |
| | 2,5- | | | |
| | diacyl | | | |
| 26 | Trans- | Me2 | 8.73 | 2.75 |
| | 1,4- | | | |
| | dioxane- | | | |
| | 2,5- | | | |
| | diacyl | | | |
| Reference | Trans- | None | 18.9 | 8.4 |
| | cyclo- | | | |
| | hexane | | | |
| | 1,4- | | | |
| | diacyl | | | |
| 3 | Trans- | 2,5,8,11,14,17,20,23- | 11.3 | 4.5 |
| | cyclo- | octaoxahexacosan- | | |
| | hexane | 26-yl | | |
| | 1,4- | | | |
| | diacyl | | | |
| 17 | Trans- | Me2 | 12.6 | 5.2 |
| | cyclo- | | | |
| | hexane | | | |
| | 1,4- | | | |
| | diacyl | | | |
| 40 | Trans- | Glutaric acid | 3.2 | 0.73 |
| | cyclo- | | | |
| | hexane | | | |
| | 1,4- | | | |
| | diacyl | | | |
| 43 | Trans- | 2-(carboxymethoxy) | 3.36 | 0.26 |
| | cyclo- | acetyl | | |
| | hexane | | | |
| | 1,4- | | | |
| | diacyl | | | |
| 5 | +C4 + | Me2 | 6.58 | 1.76 |
| | NH + PE | | | |
| | G2+ | | | |
| 6 | 21 atoms | Me2 | 4.87 | 0.75 |
| 9 | GLY- | Me2 | 9.05 | 2.49 |
| | PEG2 | | | |
| 15 | PEG7 | Me2 | 5.72 | 0.31 |
| 20 | PEG9 | 2-(carboxymethoxy) | 0.63 | 2.93 |
| | | acetyl | | |
| 24 | PEG9 | Carbamoyl | 2.3 | 0.25 |
| 37 | PEG9 | Ac | 2.71 | 0.5 |
| 10 | PEG11 | Me2 | 7.58 | 0.3 |
| 18 | PEG11 | 2-(carboxymethoxy) | 0.03 | 0.99 |
| | | acetyl | | |
| 21 | PEG11 | Carbamoyl | 2.93 | 0.14 |
| 19 | PEG13 | 2-(carboxymethoxy) | 0.75 | 0.08 |
| | | acetyl | | |
| 22 | PEG13 | Carbamoyl | 1.02 | 0.69 |

Purity quantification was conducted on Waters H Class UPLC as follows: Mobile phase A: [0.1M NaClO4, 0.05% HClO4]/EtOH: 95:5; Mobile phase B: Acetonitrile; Flow rate: 0.3 mL/min; Column: Waters BEH300 C18, 1.7 μm, 2.1×150 mm, part #186003687; Detection: Absorbance at 214 nm for both purity and concentration; Column temperature: 35° C.; Sample injection volume: 6 μL (target ˜6.0 μg); Standard injection is 6 μl (˜6.0 μg); Autosampler temperature: 5° C.;

### Gradient Pump Mode

| T (min) | % A | % B |
|---|---|---|
| 0 | 80 | 20 |
| 7 | 63 | 37 |
| 30 | 61 | 39 |
| 35 | 20 | 80 |
| 40 | 20 | 80 |
| 40.1 | 80 | 20 |
| 44 | 80 | 20 |

HMW quantification by size exclusion chromatography was conducted on Agilent Technologies 1200 Series HPLC as follows: Mobile Phase: 1 g/L L-arginine in water: Glacial Acetic Acid: Acetonitrile (65:15:20 v/v), isocratic; Column: 7.8×300 mm, 3.5 μm, Waters Insulin HMWP, Part No. WAT201549; Wavelength: 280 nm (with 5 nm bandwidth for diode array detector (DAD) Flow Rate: 1.0 mL/min; Run Time—20 min; Column Temperature—Ambient; Sample Loop: 100 μL; Sample Temperature—6±3° C.; Injection Amount—200 μg for protein.

### Example 46

The glucose lowering effect of Dimers 4, 12, 13, 21, 24, 35, 36, 42, and 43 was compared to that of RHI in Diabetic Yucatan miniature pigs (Diabetic minipigs) as follows.

Yucatan minipigs were rendered Type 1 diabetic by Alloxan injections following a proprietary protocol developed by Sinclair Research Center (Auxvasse, MO). Induction is considered successful if basal glucose levels exceed 150 mg/dL. D minipigs with plasma glucose levels of approximately 300 mg/dl were utilized in these experiments.

Male Yucatan minipigs, instrumented with two Jugular vein vascular access ports (VAP), were used in these studies. On the day of the study after an overnight fast, minipigs were placed in slings, and VAPs were accessed for infusion and sampling. At t=0 min, and after collecting two baseline blood samples for plasma glucose measurement (t=−30 minutes and t=0 minutes), minipigs were administered Humulin (recombinant human insulin, RHI) or insulin dimer (i.e., 4, 12, 13, 21, 24, 35, 36, 42, or 43) as a single bolus IV, at 0.69 nmol/kg. Humulin and immediately preceding aforementioned insulin dimers were formulated at 69 nmol/ml in a buffer containing Glycerin, 16 mg/mL; Metacresol, 1.6 mg/mL; Phenol, 0.65 mg/mL; Anhydrous Sodium Phosphate, Dibasic, 3.8 mg/mL; pH adjusted to 7.4 with HCl. After dosing, sampling continued for 480 minutes; time points for sample collection were −30 min, 0 min, 8 min, 15 min, 30 min, 45 min, 60 min, 90 min, 120 min, 150 min, 180 min, 210 min, 240 min, 270 min, 300 min, 330 min, 360 min, 420 min, 480 min. Blood was collected in K3-EDTA tubes, supplemented with 10 μg/mL aprotinin, and stored on ice until processing, which occurred within 30 minutes of collection. After centrifugation at 3000 rpm, 4° C., for 8 min, plasma was collected and aliquoted for glucose measurement using a Beckman Coulter AU480 Chemistry analyzer and for compound levels measurement.

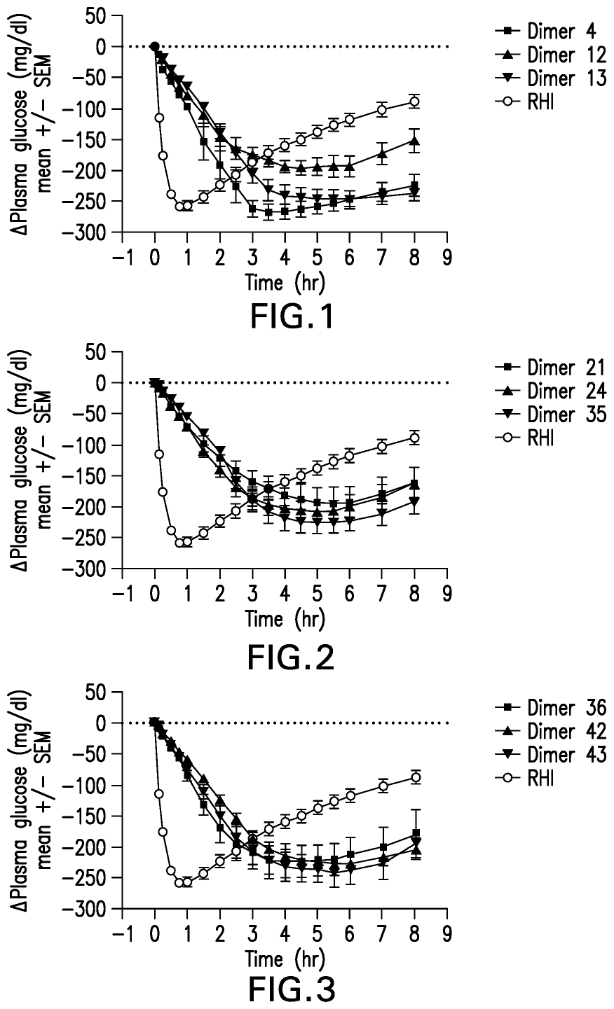

The results are shown in FIGS. 1-3. The results are presented as the change of glucose at any given time point to time 0 and show that the insulin dimers present less risk of promoting hypoglycemia than RHI.

Below are representable sequences useful in the claimed invention.

Table of Sequences

| SEQ | | |
|---|---|---|
| ID | | |
| NO: | Description | Sequence |
| 1 | Homo sapiens insulin A chain | GIVEQCCTSICSLYQLENYCN |
| 2 | Homo sapiens insulin B chain | FVNQHLCGSHLVEALYLVCGERGFFYTPKT |
| 3 | Artificial sequence insulin A chain | GX₂X₃EQCCX₈SICSLYQLX₁₇NX₁₉CX₂₃ |
| | X₂ is isoleucine or threonine; | |
| | X₃ is valine, glycine, or leucine; | |
| | X₈ is threonine or histidine; | |
| | X₁₇ is glutamic acid or glutamine; | |
| | X₁₉ is tyrosine, 4-methoxy- | |
| | phenylalanine, alanine, or 4-amino | |
| | phenylalanine; | |
| | X₂₃ is asparagine or glycine; | |
| 4 | Artificial sequence insulin B chain | X₂₅LCGX₂₉X₃₀LVEALYLVCGERGFX₂₇Y |
| | X₂₅ is histidine or threonine; | TX₃₁X₃₂ |
| | X₂₇ is phenylalanine or aspartic | |
| | acid; | |
| | X₂₉ is alanine, glycine or serine; | |
| | X₃₀ is histidine, aspartic acid, | |
| | glutamic acid, homocysteic acid, or | |
| | cysteic acid; | |
| | X₃₁ aspartic acid, proline or lysine | |
| | X₃₂ is proline or lysine, with the | |
| | proviso that at least one of X₃₁ or | |
| | X₃₂ is lysine | |
| 5 | Artificial sequence insulin B chain | X₂₂VNQX₂₅X₂₆CGX₂₉X₃₀LVEALYLVCG |
| | X₂₂ is phenylalanine or desamino- | ERGFX₂₇YTX₃₁X₃₂X₃₃X₃₄X₃₅ |
| | phenylalanine; | |
| | X₂₅ is histidine or threonine; | |
| | X₂₆ is glycine or leucine; | |
| | X₂₇ is phenylalanine or aspartic | |
| | acid; | |
| | X₂₉ is alanine, glycine, or serine; | |
| | X₃₀ is histidine, aspartic acid, | |
| | glutamic acid, homocysteic acid, or | |
| | cysteic acid; | |
| | X₃₁ is aspartic acid, proline, or | |
| | lysine; | |
| | X₃₂ is lysine or proline; | |
| | X₃₃ is threonine, alanine, or absent; | |
| | X₃₄ is arginine or absent; and | |
| | X₃₅ is arginine or absent; | |
| | With the proviso at least one of | |
| | X₃₁ or X₃₂ is lysine | |
| 6 | Artificial sequence | FVNQHLCGSHLVEALYLVCGERGFFYTKPT |
| | insulin lispro B chain | |
| 7 | Artificial sequence | GIVEQCCTSICSLYQLENYCG |
| | insulin glargine A chain | |
| 8 | Artificial sequence | FVNQHLCGSHLVEALYLVCGERGFFYTPK |
| | Insulin glargine B chain | TRR |
| 9 | Artificial sequence | FVNQHLCGSHLVEALYLVCGERGFFYTD |
| | Insulin aspart B chain | KT |
| 10 | Artificial sequence | FVNQHLCGSHLVEALYLVCGERGFFYTPK |
| | B:des30 | |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. An insulin dimer comprising:
a first B29 Lys of a first insulin heterodimer molecule having a first A-chain polypeptide and first B-chain polypeptide and a second B29 Lys of a second insulin heterodimer having a second A-chain polypeptide and second B-chain polypeptide conjugated together by a bifunctional linker selected from the group consisting Linker 1, Linker 2, Linker 3, Linker 4, Linker 5, Linker 6, Linker 7, Linker 8, Linker 9, Linker 10, Linker 11, Linker 12, Linker 13, Linker 14, Linker 15, and Linker 16, wherein at least one of the B-chain polypeptides is conjugated at its N-terminal amino acid to a capping group, or at least one of the N-terminal amino acids of the first insulin heterodimer molecule are conjugated to a capping group or at least one of N-terminal amino acids of the first insulin heterodimer and/or second insulin heterodimer are conjugated to a capping group.

2. The insulin dimer of claim 1 wherein the capping group is a linear or branched C₁₋₆ alkyl, or has the general formula RC(O)—, where R is:
a) a peptide,
b) PEG,
c) linear or branched C₁₋₆ alkyl chain,
d) R′NH—, or
e) R′O—,
wherein R′ is H (when R is R′NH—), peptide, PEG, or linear or branched alkyl chain, and wherein each said peptide, PEG and linear or branched alkyl may be unsubstituted or substituted with 1 to 3 groups selected from amino-, phosphono-, hydroxy-, carboxylic acid, amino acid, PEG, and saccharides.

3. The insulin dimer of claim 1, wherein the capping group is
a) dimethyl,
b) isobutyl, or
c) RC(O)— which is selected from acetyl, phenylacetyl, methoxy acetyl, 2-(carboxymethoxy)acetyl, 2-[bis(carboxymethylamino)]acetyl, carbamoyl, N-alkyl carbamoyl, glutaryl, trifluoroacetyl, glycyl, aminoethylglucose (AEG), AEG-C6, PEG1, PEG2, PEG3, PEG4, PEG5, PEG8, PEG24, and alkoxycarbonyl.

4. The insulin dimer of claim 1, wherein the capping group is selected from acetyl, phenylacetyl, carbamoyl, N-alkyl carbamoyl, methoxy acetyl, 2-(carboxymethoxy)acetyl, glycine, aminoethylglucose (AEG), AEG-C6, PEG1, PEG2, PEG8, N-dimethyl, isobutyl, and alkoxycarbonyl.

5. The insulin dimer of claim 1, wherein the capping group is selected from capping groups Capping Group No. 1, 2, 3, 4, 5, 6, 7, and 8.

6. The insulin dimer of claim 1, wherein the first insulin and the second insulin heterodimers are independently native human insulin, insulin lispro, insulin aspart, desB30 insulin, or insulin glargine.

7. The insulin dimer of claim 1, wherein each A-chain polypeptide independently comprises the amino acid sequence GX₂X₃EQCCX₈SICSLYQLX₁₇NX₁₉CX₂₃ (SEQ ID NO:3) and each B-chain polypeptide independently comprises the amino acid sequence X₂₅LCGX₂₉X₃₀LVEALYLVCGERGFX₂₇YTX₃₁X₃₂ (SEQ ID NO:4) or X₂₂VNQX₂₅X₂₆CGX₂₉X₃₀LVEALYLVCGERGFX₂₇YTX₃₁X₃₂X₃₃X₃₄X₃₅ (SEQ ID NO: 5) wherein X₂ is isoleucine or threonine; X₃ is valine, glycine, or leucine; X₈ is threonine or histidine; X₁₇ is glutamic acid or glutamine; X₁₉ is tyrosine, 4-methoxy-phenylalanine, alanine, or 4-amino phenylalanine; X₂₃ is asparagine or glycine; X₂₂ is or phenylalanine and desamino-phenylalanine; X₂₅ is histidine or threonine; X₂₆ is leucine or glycine; X₂₇ is phenylalanine or aspartic acid; X₂₉ is alanine, glycine, or serine; X₃₀ is histidine, aspartic acid, glutamic acid, homocysteic acid, or cysteic acid; X₃₁ is aspartic acid, proline, or lysine; X₃₂ is lysine or proline; X₃₃ is threonine, alanine, or absent; X₃₄ is arginine or absent; and X₃₅ is arginine or absent; with the proviso at least one of X₃₁ and X₃₂ is lysine.

8. The insulin dimer of according to claim 1, wherein each A-chain polypeptide comprises the amino acid sequence GIVEQCCTSICSLYQLENYCN (SEQ ID NO: 1) and each B chain comprises amino acid sequence FVNQHLCGSH LVEALYLVCGERGFFYTPKT (SEQ ID NO: 2).

9. A composition comprising:
a first insulin or insulin analog heterodimer and a second insulin or insulin analog heterodimer each heterodimer having an A-chain polypeptide and a B-chain polypeptide, wherein the A-chain polypeptide and the B-chain polypeptide are linked together through interchain disulfide bonds;
wherein the first and second insulin or insulin analog heterodimers are covalently linked together through a linking moiety joining the side chain of an amino acid at or near the carboxy terminus of the two respective B-chain polypeptides;
wherein the linking moiety is selected from the group consisting of Linker 1, Linker 2, Linker 3, Linker 4, Linker 5, Linker 6, Linker 7, Linker 8, Linker 9, Linker 10, Linker 11, Linker 12, Linker 13, Linker 14, Linker 15, and Linker 16;
wherein the insulin is human insulin and the insulin analog is selected from insulin lispro, insulin aspart, and insulin glargine; and
wherein at least one amino terminus of the B-chain polypeptides of the first insulin polypeptide and/or second insulin polypeptide is covalently linked to a capping group.

10. The composition of claim 9, wherein the first and second insulin or insulin analog heterodimers are the same.

11. The composition of claim 9, wherein the linking moiety covalently links the first insulin or insulin analog heterodimer and the second insulin or insulin analog heterodimer via the epsilon amino group of a lysine residue at or near the carboxy terminus of their respective B-chain polypeptides.

12. The composition of claim 9, wherein the capping group is a linear or branched C₁₋₆ alkyl, or has the general formula RC(O)—, where R is:
a) a peptide,
b) PEG,
c) linear or branched C₁₋₆ alkyl chain,
d) R′NH—, or
e) R′O—,
wherein R′ is H (when R is R′NH—), peptide, PEG, or linear or branched alkyl chain, and wherein each said peptide, PEG and linear or branched alkyl may be unsubstituted or substituted with 1 to 3 groups selected from amino-, phosphono-, hydroxy-, carboxylic acid, amino acid, PEG, and saccharides.

13. The composition of claim 9, wherein the capping group is selected from Capping Group No. 1, 2, 3, 4, 5, 6, 7, and 8.

14. A composition comprising an insulin dimer selected from the group consisting of

| Dimer | |
|---|---|
| # | Structure |
| 1 | 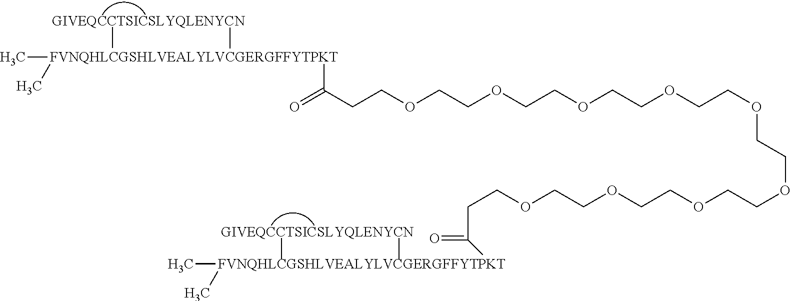 |
| 2 | 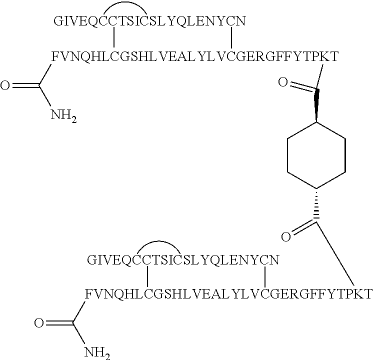 |
| 3 | 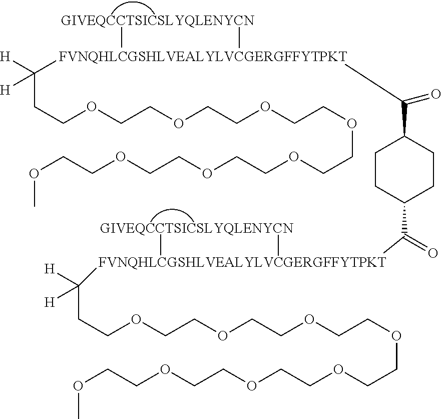 |
| 4 | 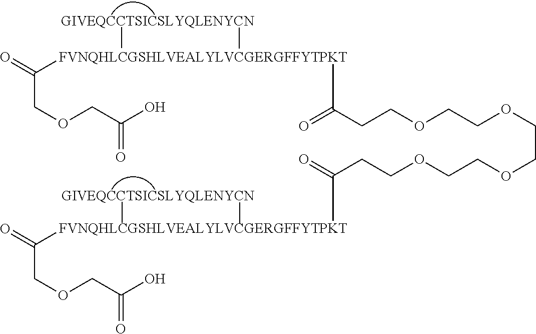 |
| 5 | 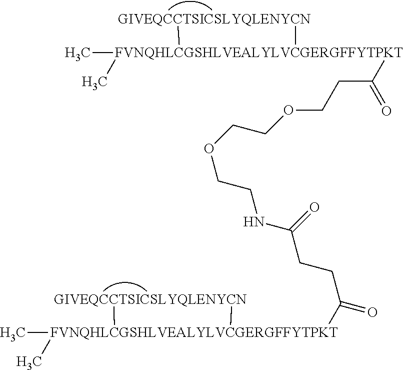 |
| 6 | 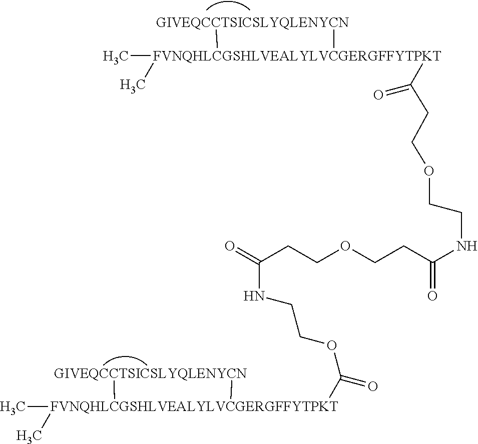 |
| 7 | 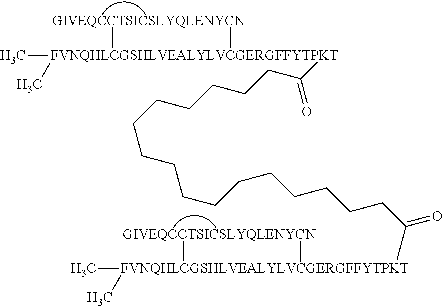 |
| 8 | 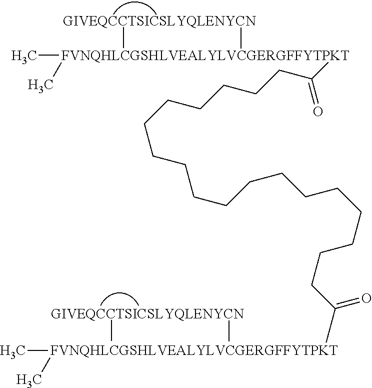 |
| 9 | 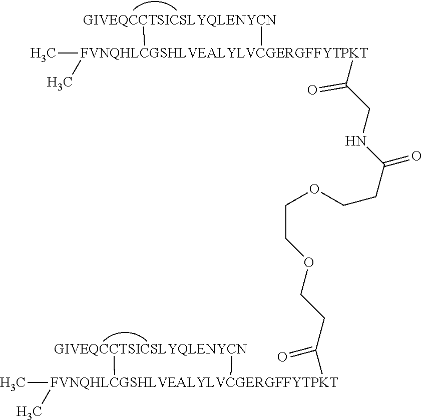 |
| 10 | 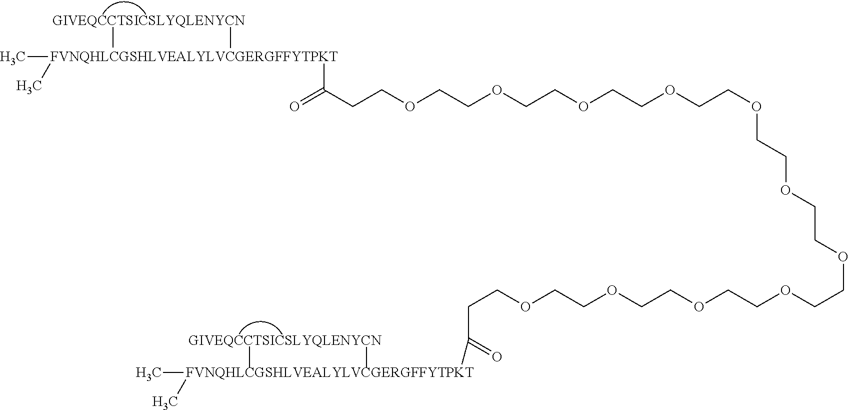 |
| 11 | 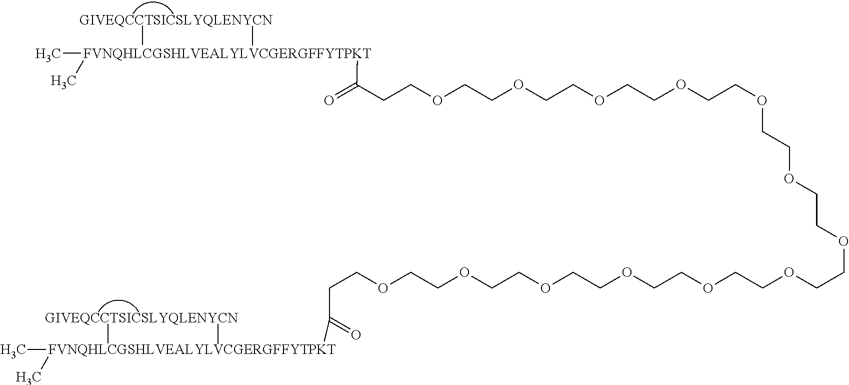 |
| 12 | 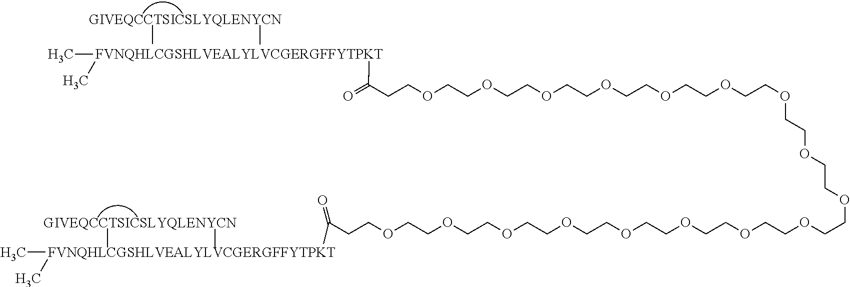 |
| 13 | 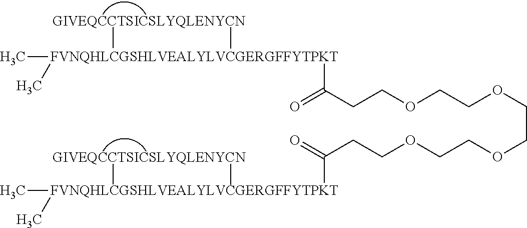 |
| 14 | 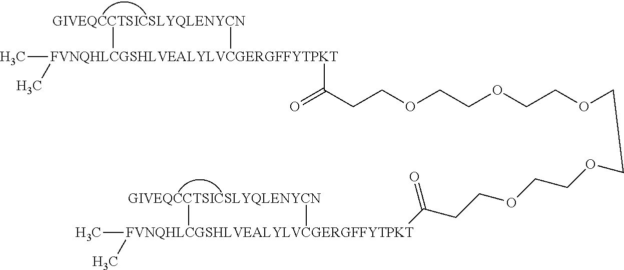 |
| 15 | 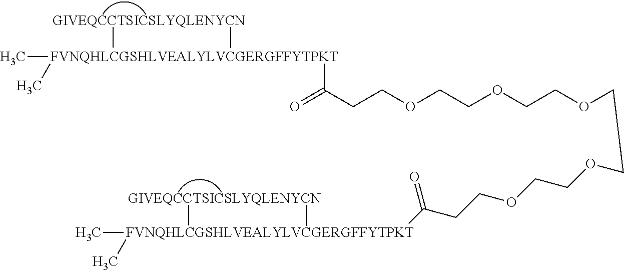 |
| 16 | 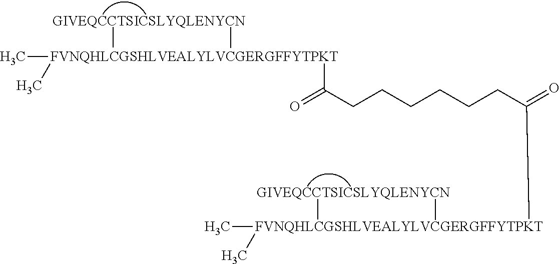 |
| 17 | 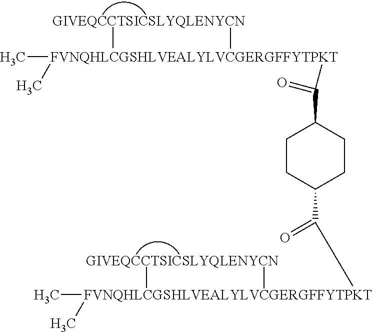 |
| 18 | 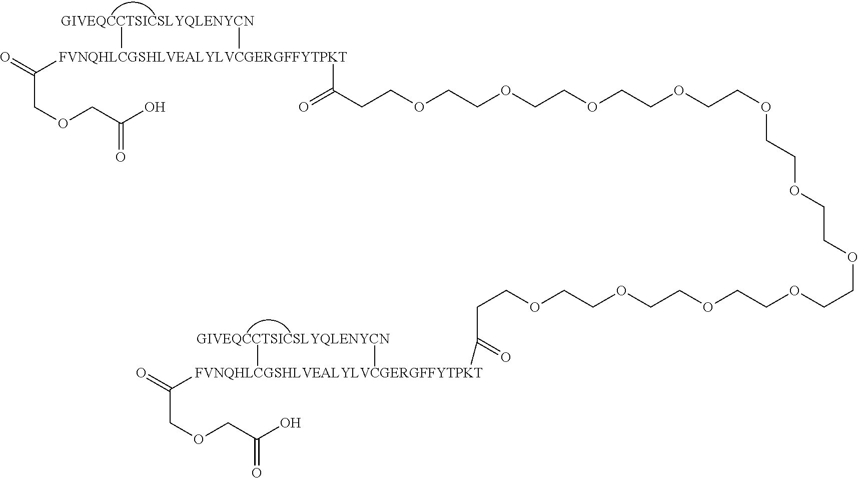 |
| 19 | 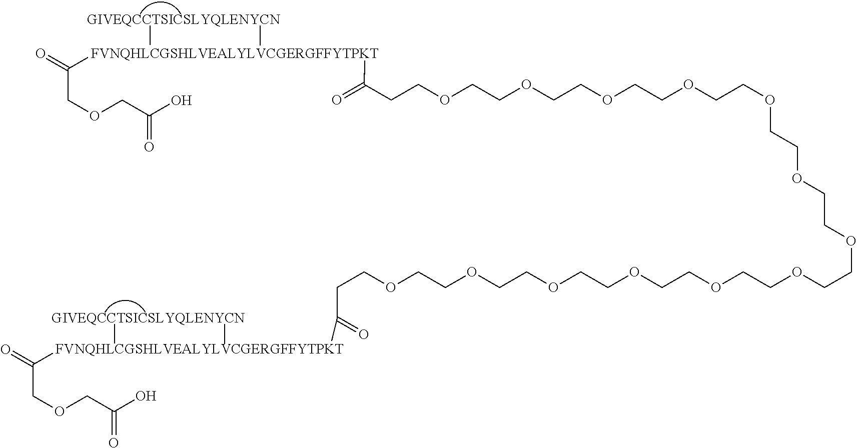 |
| 20 | 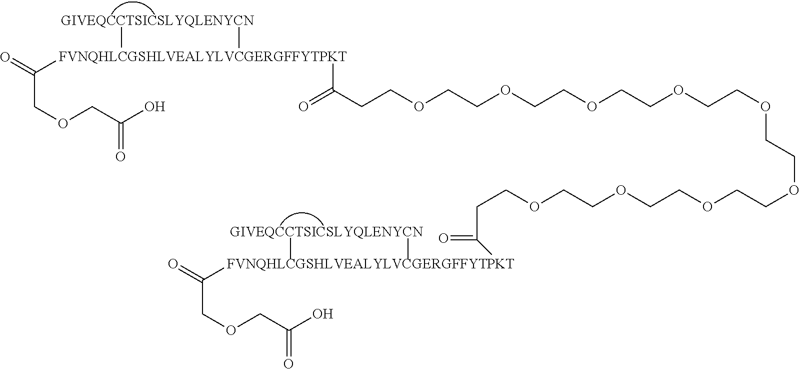 |
| 21 | 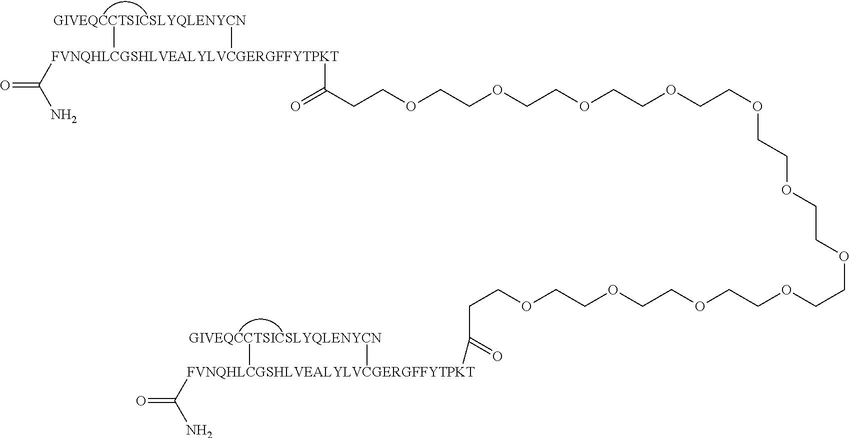 |
| 22 | 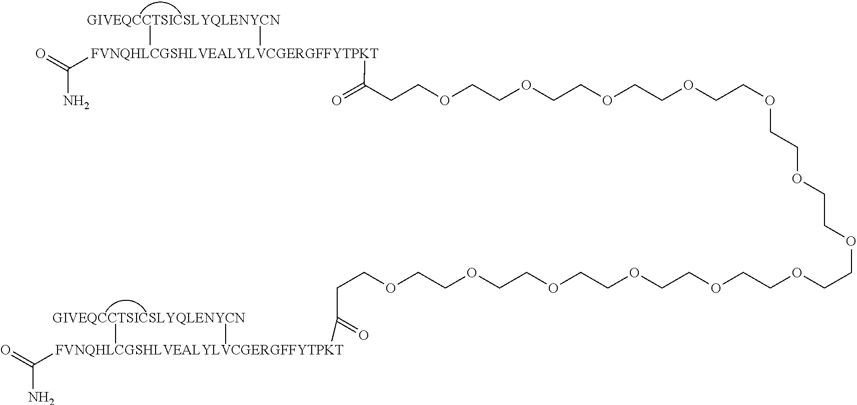 |
| 23 | 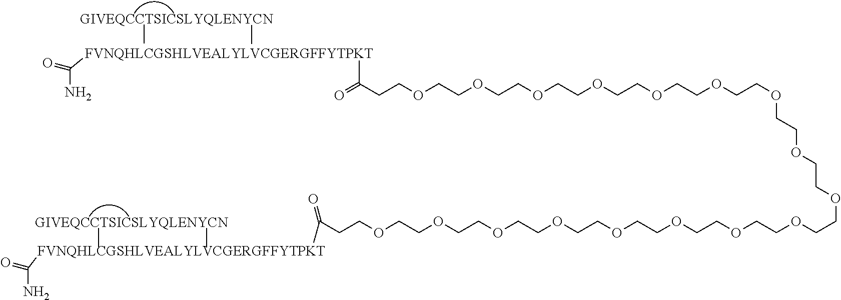 |
| 24 | 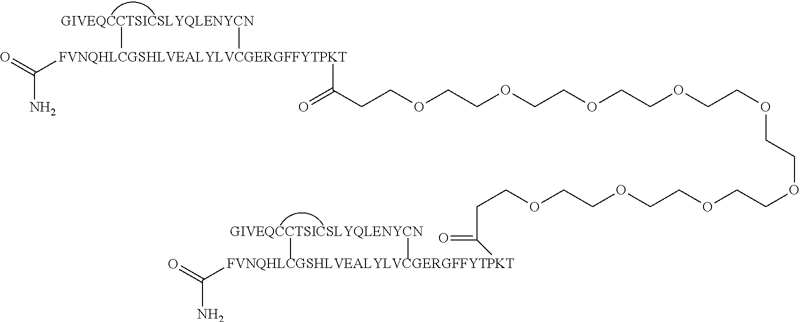 |
| 25 | 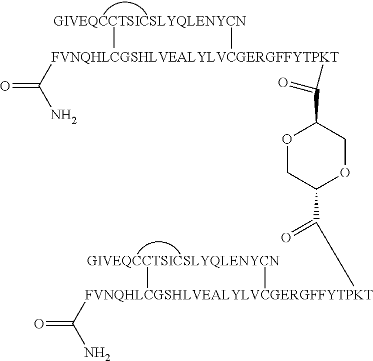 |
| 26 | 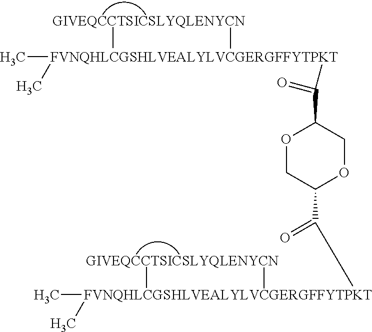 |
| 27 | 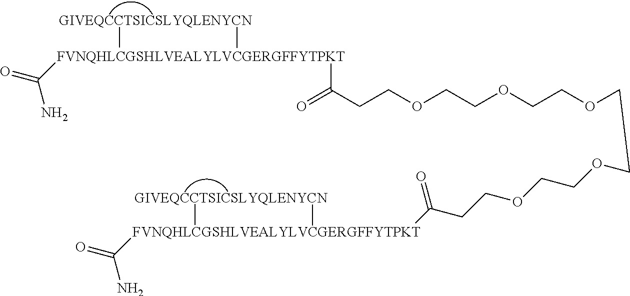 |
| 28 | 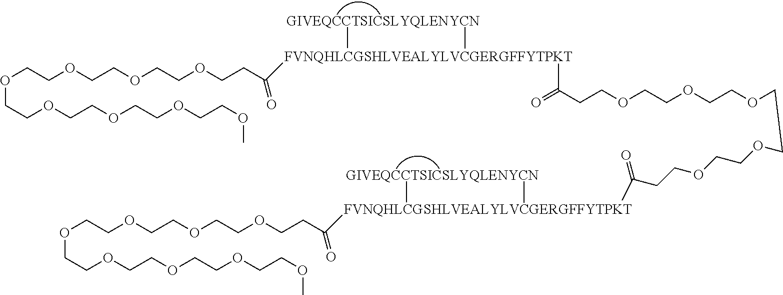 |
| 29 | 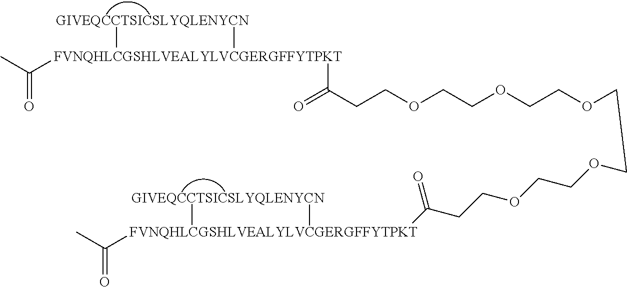 |
| 30 | 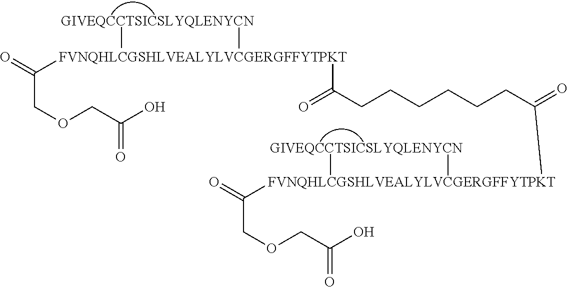 |
| 31 | 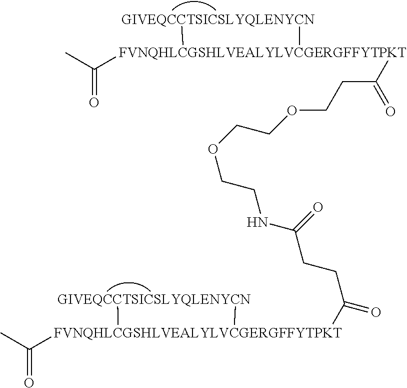 |
| 32 | 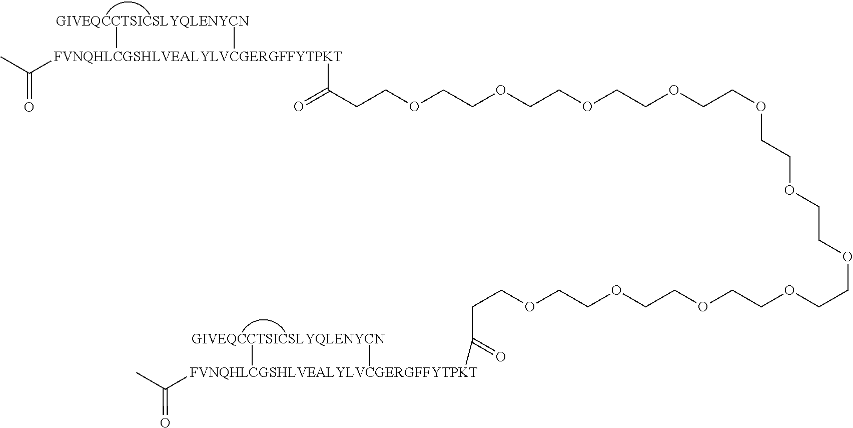 |
| 33 | 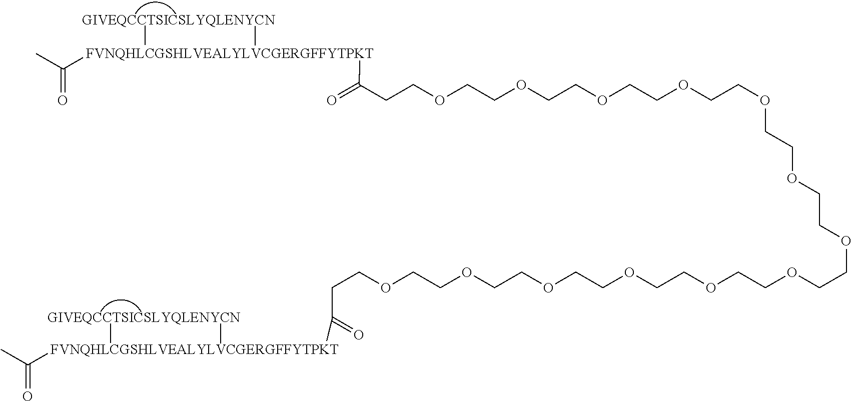 |
| 34 | 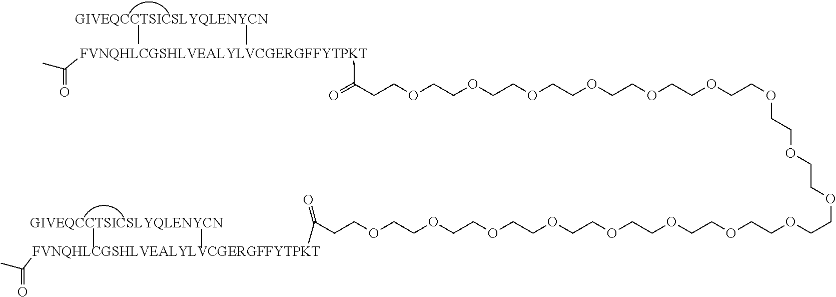 |
| 35 | 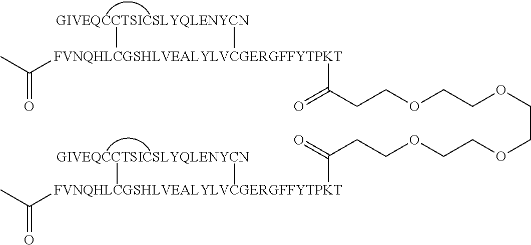 |
| 36 | 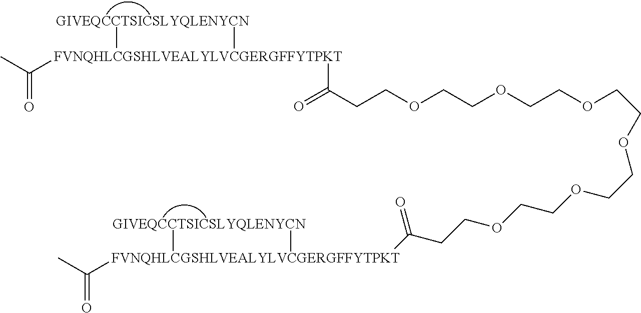 |
| 37 | 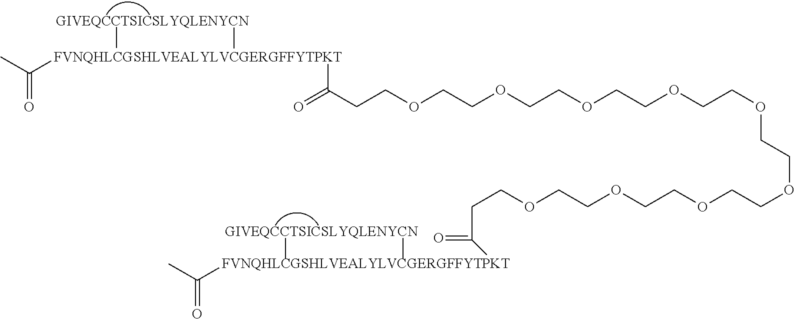 |
| 38 | 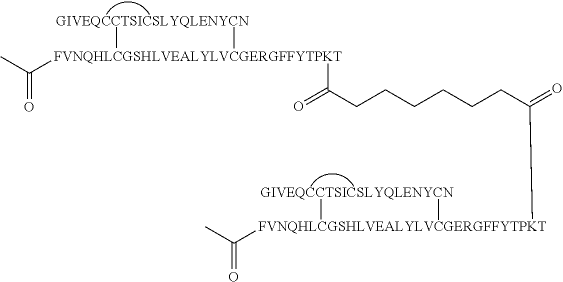 |
| 39 | 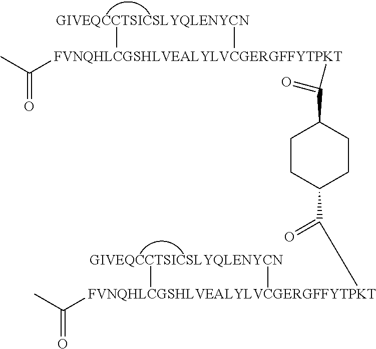 |
| 40 | 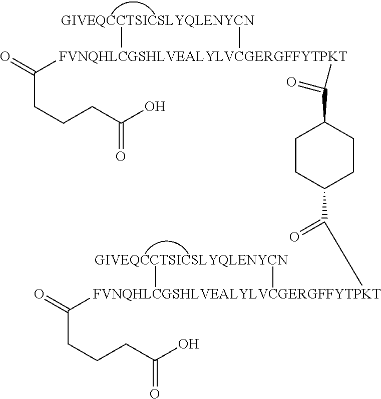 |
| 41 | 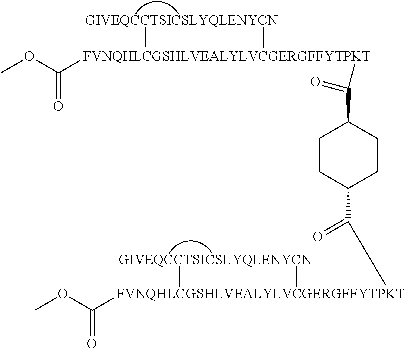 |
| 42 | 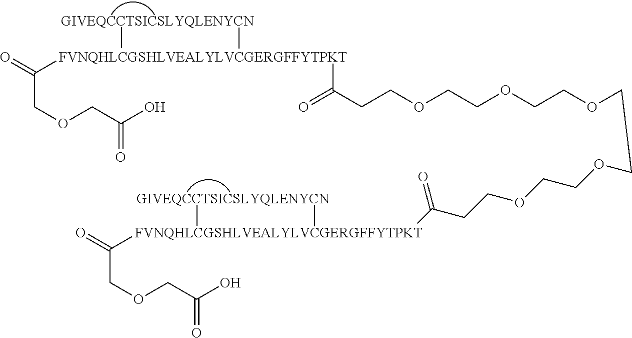 |
| 43 | 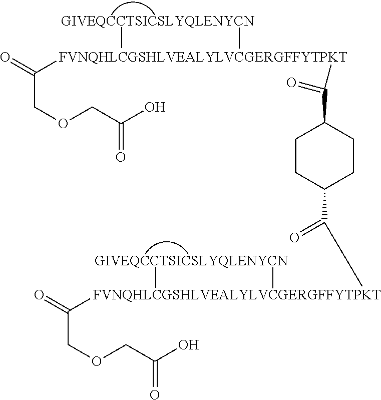 |

wherein the disulfide linkages are located between the Cys₆ and Cys₁₁ residues of the A-chain polypeptide and between the Cys₇ and Cys₂₀ of the A-chain to the Cys₇ and Cys₁₉ of the B-chain polypeptide, respectively; wherein the linking moieties are covalently linked to the epsilon amino acid of the lysine residue wherein the A-chain polypeptide for Dimers 1-43 has the amino acid sequence shown in SEQ ID NO:1; the B-chain polypeptide Dimers 1-43 has the amino acid sequence shown in SEQ ID NO:2, wherein SEQ ID NO:1 and SEQ ID NO: 2 as depicted by Formula I:

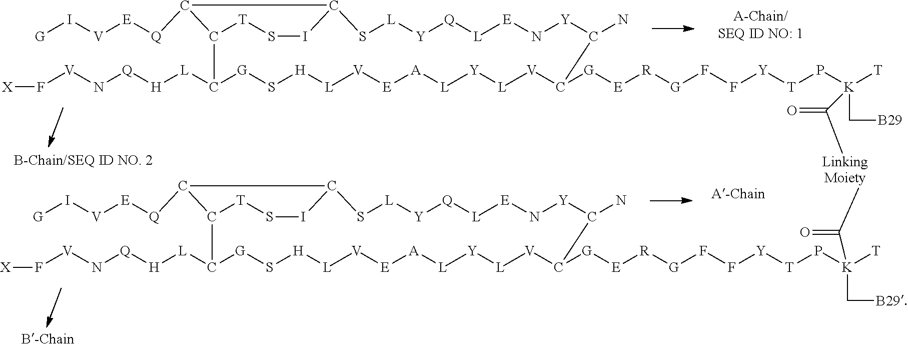

15. The composition of claim 14, wherein the composition further comprises a pharmaceutically acceptable carrier.

16. The composition of claim 14 wherein the composition further comprises a GLP-1 receptor agonist.

17. A method for treating diabetes comprising administering to an individual with diabetes a therapeutically effective amount of a composition comprising the insulin receptor partial agonist of claim 1.

18. The method of claim 17, wherein the diabetes is Type 1 diabetes, Type 2 diabetes, or gestational diabetes.

19. A composition for the treatment of diabetes comprising the insulin receptor partial agonist of claim 1.

20. The composition of claim 19, wherein the diabetes is Type 1 diabetes, Type 2 diabetes, or gestational diabetes.

21. (canceled)

22. (canceled)


SEQUENCE LISTING (10 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 21 aa, Artificial Sequence
  insulin A chain
  MISC_FEATURE (2)..(2): X2 = isoleucine or threonine
  MISC_FEATURE (3)..(3): X3=  valine, glycine, or leucine;
  MISC_FEATURE (8)..(8): X8=threonine or histidine
  MISC_FEATURE (17)..(17): X17=  glutamic acid or glutamine
  MISC_FEATURE (19)..(19): X19= tyrosine, 4-methoxy-phenylalanine, alanine, or 4-amino phenylalanine
  MISC_FEATURE (21)..(21): X23=  asparagine or glycine
GXXEQCCXSICSLYQLXNXCX

SEQ ID NO: 4 - protein, 25 aa, Artificial Sequence
  insulin B chain
  MISC_FEATURE (1)..(1): X25 is histidine or threonine
  MISC_FEATURE (5)..(5): X29 is alanine, glycine or serine;
  MISC_FEATURE (6)..(6): X30 is histidine, aspartic acid, glutamic acid, homocysteic acid, or cysteic acid
  MISC_FEATURE (21)..(21): X27 is phenylalanine or aspartic acid
  MISC_FEATURE (24)..(24): X31 aspartic acid, proline or lysine
  MISC_FEATURE (25)..(25): X32 is proline or lysine, with the proviso that at least one of X31 or X32 is lysine
XLCGXXLVEALYLVCGERGFXYTXX

SEQ ID NO: 5 - protein, 32 aa, Artificial Sequence
  insulin chain
  MISC_FEATURE (1)..(1): X22 = is phenylalanine or desamino-phenylalanine
  MISC_FEATURE (5)..(5): X25 =is histidine or threonine
  MISC_FEATURE (6)..(6): X26=is glycine or leucine
  MISC_FEATURE (9)..(9): X29 =is alanine, glycine, or serine
  MISC_FEATURE (10)..(10): X30=is histidine, aspartic acid, glutamic acid, homocysteic acid, or cysteic acid
  MISC_FEATURE (25)..(25): X27 is phenylalanine or aspartic acid
  MISC_FEATURE (28)..(28): X31 is aspartic acid, proline, or lysine
  MISC_FEATURE (29)..(29): X32= is lysine or proline
  MISC_FEATURE (30)..(30): X33= is threonine, alanine, or absent
  MISC_FEATURE (31)..(31): X34= arginine or absent
  MISC_FEATURE (32)..(32): X35 is arginine or absent; With the proviso at least one of X31 or X32 is lysine
XVNQXXCGXXLVEALYLVCGERGFXYTXXXXX

SEQ ID NO: 6 - protein, 30 aa, Artificial Sequence
  insulin lispro B chain
FVNQHLCGSHLVEALYLVCGERGFFYTKPT

SEQ ID NO: 7 - protein, 21 aa, Artificial Sequence
  insulin glargine A chain
GIVEQCCTSICSLYQLENYCG

SEQ ID NO: 8 - protein, 32 aa, Artificial Sequence
  Insulin glargine B chain
FVNQHLCGSHLVEALYLVCGERGFFYTPKTRR

SEQ ID NO: 9 - protein, 60 aa, Artificial Sequence
  Insulin aspart B chain
FVNQHLCGSHLVEALYLVCGERGFFYTDKTFVNQHLCGSHLVEALYLVCGERGFFYTDKT

SEQ ID NO: 10 - protein, 29 aa, Artificial Sequence
  Bdes30
FVNQHLCGSHLVEALYLVCGERGFFYTPK